(19) United States
(12) Patent Application Publication
(10) Pub. No.: US 2024/0383827 A1
(43) Pub. Date: Nov. 21, 2024

(54) DIFLUOROCARBENE RADIOSYNTHESIS

(71) Applicant: Oxford University innovation Limited, Oxfordshire (GB)

(72) Inventors: Veronique Emilie Bernardine Marie Gouverneur, Oxfordshire (GB); Claudio Flavio Meyer, Oxfordshire (GB); Jeroen Bart I. Sap, Oxfordshire (GB)

(73) Assignee: Oxford University innovation Limited, Oxfordshire (GB)

(21) Appl. No.: 18/694,241

(22) PCT Filed: Sep. 22, 2022
(86) PCT No.: PCT/GB2022/052389

§ 371 Date: Mar. 21, 2024

(30) Foreign Application Priority Data
Sep. 23, 2021 (GB) 2113561.1

(51) Int. Cl. C07C 17/361; A61K 31/025; A61K 31/085; A61K 31/095; A61K 31/11; A61K 31/122; A61K 31/381; A61K 31/4184; A61K 31/4192; A61K 31/5375

(52) U.S. Cl. CPC C07C 17/361; A61K 31/025; A61K 31/085; A61K 31/095; A61K 31/11; A61K 31/122; A61K 31/381; A61K 31/4184; A61K 31/4192; A61K 31/5375

(57) ABSTRACT

The invention relates to a process for producing a compound comprising an ¹⁸F-difluoromethyl group or a compound comprising an ¹⁸F-difluoromethylene group, which process comprises contacting a compound comprising a nucleophilic group with ¹⁸F-difluorocarbene. The invention also relates to a compound of Formula (I). Compounds comprising an ¹⁸F-difluoromethyl group or an ¹⁸F-difluoromethylene group are also the subject of the present invention.

### FIELD OF THE INVENTION

The invention relates to a process for preparing a compound comprising an ¹⁸F-difluoromethyl group or a compound comprising an ¹⁸F-difluoromethylene group. The invention also relates to a reagent which may be used in that process. Compounds comprising an ¹⁸F-difluoromethyl group or an ¹⁸F-difluoromethylene group are also the subject of the present invention.

### BACKGROUND TO THE INVENTION

As a highly sensitive and non-invasive imaging technique, positron emission tomography (PET) is an imperative tool within the field of nuclear imaging and drug discovery. An important feature contributing to the success of PET is the availability of methodologies for the incorporation of radioisotopes into (bio)molecules of interest. Amongst the available radionuclides, fluorine-18 is one of the most commonly used due to its advantageous properties (positron energy of 0.63 MeV, t1/2=109.7 mins), and the demand for structurally complex ¹⁸F-labeled tracers continues to grow.

Today, methods for the preparation of structurally diverse radiotracers from [¹⁸F]fluoride have relied on nucleophilic substitution of alkyl electrophiles, nucleophilic aromatic substitution (SNAr) and more recently cross-coupling technologies (see Wilson, T. C. et al. A One-Pot Radio-synthesis of [¹⁸F]PARPi. J. Labelled Compd. Radiopharmaceut. 2020; Lemaire, C. et al. A new route for the synthesis of [¹⁸F] fluoroaromatic substituted amino acids: no carrier added Lp-[¹⁸F]fluorophenylalanine. Int. J. Rad. Appl. Instrum. A. 1987, 38, 1033-1038). Another compelling strategy to F-18 label substrates which bare innate nucleophilic handles such as (thio)phenols or N-heteroarenes exploits [¹⁸F]fluoride-derived alkylating reagents, most commonly [¹⁸F]fluoromethyltosylate (see Merchant, S. et al.; Aboagye, E. O. Synthesis and pre-clinical evaluation of a [¹⁸F]fluoromethyl-tanaproget derivative for imaging of progesterone receptor expression. RSC Adv. 2016, 6, 57569-57579). In contrast to the first category, which has witnessed continuous development throughout the last decade, innovation of the latter has lagged behind. Whilst ¹⁸F-alkylation strategies are typically robust and high yielding from a radiochemistry standpoint, challenges remain due to the inherent instability of the resulting fluoromethyl(thio)ethers (see Zhao, Q. et al. Direct Monofluoromethylthiolation with S-(Fluoromethyl) Benzenesulfonothioate. Angew. Chem. Int. Ed. 2017, 56, 11575-11578). The instability of these tracers leads to the release of [¹⁸F]fluoride resulting in increased bone uptake and therefore high background PET scans which exhibit a poor signal to noise ratio (Carson, R. E.; et al. Brain Uptake of the Acid Metabolites of F-18-Labeled WAY 100635 Analogs. J. Cereb. Blood Flow Metab. 2003, 23, 249-260).

To combat in vivo defluorination of fluoroalkylated tracers, radiochemists have employed tactics such as extending the length of fluoroalkyl chains or exchanging specific hydrogen atoms for deuterium (Kuchar, M. et al. Methods to increase the metabolic stability of ¹⁸F-radiotracers. Molecules. 2015, 20, 16186-16220). Whilst in several cases, the in vivo properties of such tracers can be improved through these strategies, deuteration typically does not alleviate defluorination in its entirety. Furthermore, extension of the fluoroalkyl spacer generates an increased steric bulk, which may augment the properties of the radiotracer (Zafrani, Y. et al. Difluoromethyl Bioisostere: Examining the “Lipophilic Hydrogen Bond Donor” Concept. J. Med. Chem. 2017, 60, 797-804). Whilst in ¹⁹F chemistry difluoromethyl(thio)ethers have emerged as stable bioisosteres of methoxy groups, protecting drugs against undesired metabolism, ¹⁸F-radiolabelling technologies to access the ¹⁸F-difluoromethyl motif remains underdeveloped, typically lacking generality and application to complex ¹⁸F-labeled tracers.

Difluoromethyl(thio)ethers are usually prepared by the net insertion of difluorocarbene (DFC) into the (thio)phenol X—H bond (X═O, S) (Ni, C.; Hu, J. Recent advances in the synthetic application of difluorocarbene. Synthesis 2014, 46, 842-863; Dilman, A. D.; Levin, V. V. Difluorocarbene as a building block for consecutive bond-forming reactions. Acc. Chem. Res. 2018, 51, 1272-1280). DFC is an extremely versatile reactive intermediate in organic synthesis, and the F-18 labelling of difluorocarbene ([¹⁸F]DFC) itself would be a promising endeavour in the field of F-18 radiochemistry. Whilst the F-18 labelling of this putative intermediate allows for a general route towards the radiosynthesis of [¹⁸F]difluoromethyl(thio)ethers, its applications likely stretch way beyond these motifs.

The ¹⁸F-trifluoromethylthiolation of alkyl halides by the combination of [¹⁸F]fluoride with elemental sulfur and ¹⁹F-difluorocarbene has been demonstrated (Zheng, J. et al., S. H. Difluorocarbene-Derived Trifluoromethylthiolation and [¹⁸F]Trifluoromethylthiolation of Aliphatic Electrophiles. Angew. Chem. Int. Ed. 2015, 54, 13236-13240). The ¹⁸F-trifluoromethylation of aryl iodides and the radiosynthesis and application of [¹⁸F]ammonium trifluoro-methanesulfinate has also been recently disseminated (Huiban, M. et al., A broadly applicable [¹⁸F]trifluoromethylation of aryl and heteroaryl iodides for PET imaging. Nat. Chem. 2013, 5, 941-944.16). Both these transformations rely on the reaction of [¹⁸F]fluoride with [¹⁹F]DFC to produce a reagent containing a [¹⁸F]trifluoromethyl moiety in the form of a [CF₂¹⁸FSO₂]⁻ anion (Kee, C. W. et al. J. Am. Chem. Soc. 2020, 142, 1180-1185). It is that anion which is then reacted a compound to be labelled to provide an [¹⁸F]trifluoromethyl label. However, these methodologies are not suitable for providing compounds having an [¹⁸F]difluoromethyl group (—CF¹⁸FH).

Zhao et al (Zhao et al. Radiosynthesis of [¹⁸F]ArylSCF₂H using aryl boronic acids, S-(chlorofluoromethyl) benzenesulfonothioate and [¹⁸F]fluoride. CCS Chemistry 2020, 1921-1028) report the synthesis of various aromatic groups having an —CHF¹⁸F group. This is accomplished by installing a —SCFClH group then converting this to a —SCHF¹⁸F group using halogen exchange with ¹⁸F. This process is only applicable to these sulfur containing functional groups, and does not involve an ¹⁸F-difluorocarbene reagent.

Sap et al (Sap et al. Synthesis of ¹⁸F-difluoromethylarenes using aryl boronic acids, ethyl bromofluoroacetate and [¹⁸F]fluoride. Chem. Sci. 2019, 10, 3237-3241) describe the conversion of an ArB(OH)₂ precursor to ArOCHF¹⁸F using a two-step process in which the Cu-catalysed coupling is performed to provide an ArOCFHCO₂H precursor which subsequently undergoes a fluorodecarboxylation reaction in the presence of ¹⁸F to convert to the CHF¹⁸F moiety. Mn-mediated flurodecarboxylation access to [¹⁸F]ArOCF₂H is also demonstrated, but requires the presence of a carboxyl moiety to perform the transformation.

Two-step processes involving decarboxylation for the production of [¹⁸F]ArOCF₂H are also described in Khotavivattana et al (Khotavivattana et al. Synthesis and Reactivity of ¹⁸F-Labeled a, α-Difluoro-α-(aryloxy) acetic Acids. Org. Lett. 2017, 19, 568-571). Khotavivattana also reports in ¹⁸F-Labeling of Aryl-SCF₃, —OCF₃ and —OCHF₂ with [¹⁸F]Fluoride. Angew. Chem. Int. Ed. 2015, 54, 9991-9995 the use of Ag(I) to perform the halex reaction converting ArOCHFCl to [¹⁸F]ArOCF₂H. Both of these processes involve specific functional groups to start (for instance a carboxyl group adjacent to the C to which the ¹⁸F is bound, or a halogen-containing group), so lack general applicability to multiple types of nucleophilic groups.

The insertion of the radical [CHF¹⁸F] into C—H bonds to provide a [¹⁸F]CF₂H is also reported in Trump et al. Late-Stage ¹⁸F-Difluoromethyl Labeling of N-Heteroaromatics with High Molar Activity for PET Imaging. Angew. Chem. Int. Ed. 2019, 58, 13149-13154, Trump et al. Development of a general automated flow photoredox 18F-difluoromethylation of N-heteroaromatics in an AllinOne synthesizer. Org. Process Res. Dev. 2020, 24, 734-744 and in Lemos et al. Radical C—H 18F-Difluoromethylation of Heteroarenes with [¹⁸F] Difluoromethyl Heteroaryl-Sulfones by Visible Light Photoredox Catalysis. Catalysts 2020, 10, 275. However, this radical-based methodology is only applicable to CH bonds and is not suitable for addition of [¹⁸F]CF₂H to nucleophilic groups.

There therefore exists a need to develop a reaction methodology and reagents suitable for installing [¹⁸F]CF₂H at nucleophilic sites (OH, SH, NH and others). The methodology should ideally be applicable to as wide a variety of nucleophiles as possible and be conducted in a single step. Any reagents involved should be stable, easy to purify and react in a controlled an predictable fashion providing an acceptable yield of product. Further, due to the short half-life of ¹⁸F, the approaches to providing the ¹⁸F labelled compounds should be quick to perform and high yielding. Therefore, both the synthesis of the ¹⁸F-containing reagents, and their subsequent reactions with the nucleophilic groups should be straightforward, requiring as few steps as possible. Otherwise, by the time the desired ¹⁸F-labelled product were synthesized positron emission would have decayed to levels unsuitable for use in PET imaging.

### SUMMARY OF THE INVENTION

There is no report on the radiosynthesis [¹⁸F]DFC itself. Whilst isotopic exchange of ¹⁹F-DFC is a viable option, the expected low molar activity (MA) encourages a reagent-based approach (see Trump, L. et al. Late-Stage ¹⁸F-Difluoromethyl Labeling of N-Heteroaromatics with High Molar Activity for PET Imaging. Angew. Chem. Int. Ed. 2019, 58, 13149-13154; Trump, L. et al. Development of a general automated flow photoredox ¹⁸F-difluoromethylation of N-heteroaromatics in an AllinOne synthesizer. Org. Process Res. Dev. 2020, 24, 5, 734-744). The inventors have developed the first radiosynthesis of a [¹⁸F]DFC reagent and illustrate its broad utility as a reactive intermediate in F-18 radiochemistry to install ¹⁸F-difluoromethyl moieties. This reagent allows controlled [¹⁸F]DFC insertion into a variety of X—H (X═O, S, N) bonds and other nucleophiles, representing a new and valuable tool to accelerate the discovery of novel PET ligands with more stable fluorinated motifs. The novel ¹⁸F reagents may be prepared in a two-step-one pot protocol (fluorination followed by oxidation) from [¹⁸F]fluoride, allowing fast processing of ¹⁸F to useful ¹⁸F labelled products.

This discovery has opened the possibility of synthesizing a variety of [¹⁸F] labelled molecules in a one-pot fashion. In view of the number of reactions relying on difluorocarbene-type reagents, access to the first [¹⁸F]DFC reagent, has a considerable impact on the radiochemical space which is may be explored for PET applications. Specifically, it also provides an alternative entry point to access more stable analogues of ¹⁸F radiotracers. As outlined above, many existing routes rely on ¹⁸F-alkylation strategies in which the resulting alkylation products readily undergo radiodefluorination.

The present invention provides a process for producing a compound comprising an ¹⁸F-difluoromethyl group or a compound comprising an ¹⁸F-difluoromethylene group, which process comprises contacting a compound comprising a nucleophilic group with ¹⁸F-difluorocarbene.

The invention also provides a compound of formula (I)

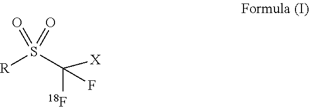

wherein X is H or Cl, preferably wherein X is H, and wherein R is a substituted or unsubstituted aryl group.

The invention also provides a compound comprising an ¹⁸F-difluoromethyl group bonded to nitrogen.

The invention also provides a compound comprising a gem-¹⁸F-difluorocyclopropane moiety.

The invention also provides a compound comprising an ¹⁸F-difluoromethyl group bonded to an alkyne.

The invention also provides a compound comprising an ¹⁸F-difluoromethyl group bonded to an oxygen atom.

The invention also provides a compound comprising an ¹⁸F-difluoromethyl group bonded to a sulfur atom.

The invention also provides a compound as described herein wherein the compound is obtainable by the process as described herein.

The invention also provides a compound as described herein for use in a method for treatment of the human or animal body by therapy or for use in a diagnostic method practised on the human or animal body.

The invention also provides a method of imaging a subject, comprising administering to the subject a compound as described herein or a pharmaceutically acceptable salt thereof, and imaging the subject by positron emission tomography (PET).

### BRIEF DESCRIPTION OF THE FIGURES

FIG. 1A shows the one-pot radiosynthesis of [¹⁸F] 1; FIG. 1B shows the divergent reactivity profile of [¹⁸F]1 under different reaction conditions; FIG. 1C shows the molar activity of various [¹⁸F] compounds where R═H, ^{t}Bu, NO₂ and Cl.

FIG. 2A shows the limitations of known methods, an exemplary strategy according to the present invention and examples of phenols, thiophenols and N-heterocycles treated with the difluorcarbene reagent [¹⁸F]1; FIG. 2B shows examples of biologically relevant molecules and radioligands treated with difluorocarbene reagent; FIG. 2C shows tandem and one-pot sequences for introducing the ¹⁸F[CF₂H] moiety. ′Reaction performed with [¹⁸F] 1-(tert-butyl)-4-((difluoromethyl)sulfonyl)benzene. \hat{ }Reaction performed with [¹⁸F]1-chloro-4-((difluoromethyl)sulfonyl)benzene. ^{a}Reaction was performed at 80° C. ^{b}Reaction was performed at 100° C.; ^{c}Reaction was performed in DMF (anhydrous, 0.3 mL) at 140° C. with NaH (0.1 mmol) in place of KOH; ^{d}Reaction performed in DMF (anhydrous, 0.3 mL) at 60° C. with NaH (0.1 mmol) in place of KOH; ^{e}Reaction performed on 0.1 mmol scale; ^{f}Urea hydrogen peroxide (1.0 equiv), boronic acid in MeCN (0.5 mL), 5 min, rt, then quench KOH_{(aq)} (25% w/w, 0.1 mL), followed by addition of [¹⁸F]ArSO₂CF₂H, 20 min, 100° C. ^{g}aryl chloride (0.2 mmol), B₂Pin₂ (0.2 mmol), KOAc (0.6 mmol), Pd₂dba₃ (0.0025 mmol), XPhos (0.01 mmol), 1,4-dioxane (400 μL), 110° C., 20 min, then filtration, volatiles removed, then Urea·hydrogen peroxide (0.2 mmol), in MeCN (0.5 mL) and DMSO (0.1 mL), 5 min, rt, then quench KOH_{(aq)} (25% w/w, 0.15 mL), followed by addition of [¹⁸F]ArSO₂CF₂H, 20 min, 100° C.

FIG. 3 shows the quantitative 19F NMR spectrum of the difluoromethylation reaction for 4-phenylphenol performed in deuterated solvent.

FIG. 4 shows the quantitative 19F NMR spectrum of the difluoromethylation reaction for 4-phenylphenol and 1-(Tert-butyl)-4-((difluoromethyl-d)sulfonyl)benzene performed in deuterated solvent.

FIG. 5 shows the quantitative ¹⁹F NMR spectrum of the difluoromethylation reaction of sodium phenoxide.

FIG. 6 shows the quantitative ¹⁹F NMR spectrum of the difluoromethylation reaction of sodium thiophenolate.

FIG. 7 shows the percentage yield (based on 4-^{t}BuPhSO₂CF₂H) as a function of equivalents of 4-^{t}BuPhSO₂CF₂H.

FIGS. 8 to 16 show the radio HPLC for the crude reaction mixture for compound 5.20d to 5.20l. General procedure 3 was followed to synthesise the compounds.

FIG. 17 shows the radio HPLC for the crude reaction mixture for compound 5.20m. General procedure 3 was performed at 100° C. to synthesise the compound.

FIGS. 18 to 32 show the radio HPLC for the crude reaction mixture for compounds 5.20n, 5.20o, 5.20p, 5.20b, 5.20q, 5.20r, 5.20s, 5.29a, 5.29b, 5.29c, 5.29d, 5.29e, 5.31a, 5.31b respectively. General procedure 3 was followed to synthesise the compounds.

FIGS. 33 and 34 show the radio HPLC for the crude reaction mixture for compounds 5.31c and 5.31d. General procedure 5 was followed to synthesise the compound.

FIG. 35 shows the radio HPLC for the crude reaction mixture for compound 5.20t. General procedure 3 was followed to synthesise the compound.

FIG. 36 shows the radio HPLC for the crude reaction mixture for compound 5.20u. General procedure 3 was followed to synthesise the compound; the reaction was performed on a 0.1 mmol scale at 100° C.

FIG. 37 shows the radio HPLC for the crude reaction mixture for compound 5.20v. General procedure 3 was performed at 100° C. to synthesise the compound.

FIG. 38 shows the radio HPLC for the crude reaction mixture for compound 5.31f. General procedure 3 was followed to synthesise the compound. Note: The regioselectivity could not be assessed from the crude radio HPLC. Likely the reaction affords two regioisomers.

FIG. 39 shows the radio HPLC for the crude reaction mixture for compound 5.31g. General procedure 5 was performed at 140° C. to synthesise the compound.

FIG. 40 shows the radio HPLC for the crude reaction mixture for compound [¹⁸F]DF-MPC-6827. General procedure 3 was performed at 100° C. to synthesise this compound.

FIG. 41 shows the radio HPLC for the crude reaction mixture for compound [¹⁸F]DF-XY. General procedure 3 was followed to synthesise the compound; the reaction was performed on a 0.1 mmol scale at 100° C.

FIG. 42 shows the radio HPLC for the crude reaction mixture for the reaction of Etoricoxib-B(OH)₂.

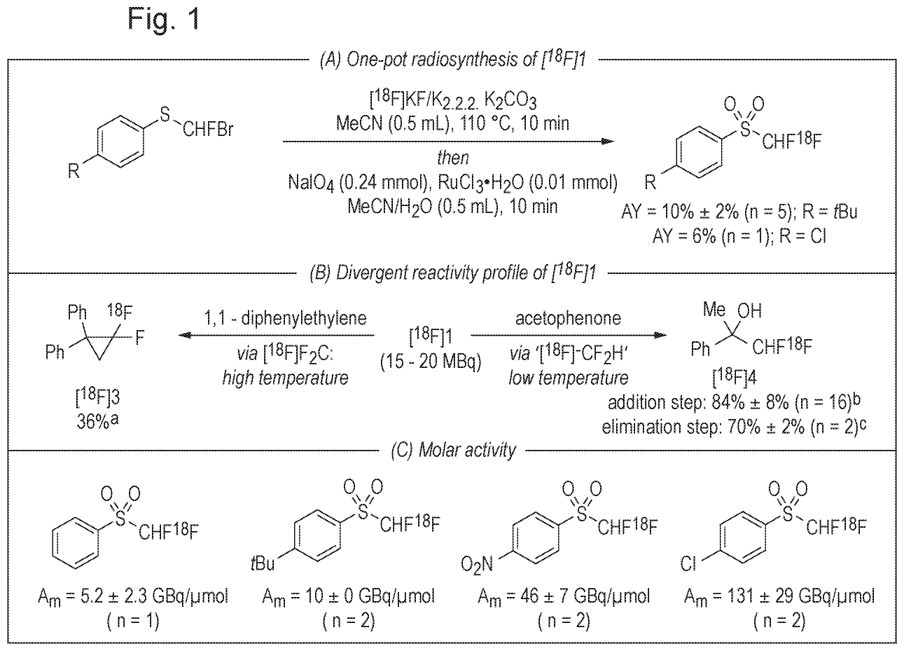

### DETAILED DESCRIPTION

### Definitions

The term “alkyl group”, as used herein, refers to a substituted or unsubstituted, straight or branched chain saturated hydrocarbon radical. Typically an alkyl group is C₁₋₂₀ alkyl, or C₁₋₁₀ alkyl, for example methyl, ethyl, propyl, butyl, pentyl, hexyl, heptyl, octyl, nonyl or decyl (including straight or branched chain isomers thereof), or C₁₋₆ alkyl, for example methyl, ethyl, propyl, butyl, pentyl or hexyl (including straight or branched chain isomers thereof), or C₁₋₄ alkyl, for example methyl, ethyl, i-propyl, n-propyl, t-butyl, s-butyl or n-butyl. When an alkyl group is substituted it typically bears one or more substituents selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Examples of substituted alkyl groups include haloalkyl, hydroxyalkyl, aminoalkyl, alkoxyalkyl and alkaryl groups. The term alkaryl, as used herein, pertains to a C₁₋₂₀ alkyl group in which at least one hydrogen atom has been replaced with an aryl group. Examples of such groups include, but are not limited to, benzyl (phenylmethyl, PhCH₂—), benzhydryl (Ph₂CH—), trityl (triphenylmethyl, Ph₃C—), and phenethyl (phenylethyl, Ph-CH₂CH₂—). Typically a substituted alkyl group carries 1, 2 or 3 substituents, for instance 1 or 2.

The term “alkenyl”, as used herein, refers to a linear or branched chain hydrocarbon radical comprising one or more double bonds. An alkenyl group may be a C₂₋₂₀ alkenyl group, a C₂₋₁₀ alkenyl group or a C₂₋₆ alkenyl group. Examples of C₂₋₂₀ alkenyl groups include those related to C₂₋₂₀ alkyl groups by the insertion of one or more double bonds. Alkenyl groups typically comprise one or two double bonds. The alkenyl groups referred to herein may be substituted or unsubstituted, as defined for alkyl groups above.

The term “alkynyl”, as used herein, refers to a linear or branched chain hydrocarbon radical comprising one or more triple bonds. An alkynyl group may be a C₂₋₂₀ alkynyl group, a C₂₋₁₀ alkynyl group a C₂₋₆ alkynyl group. Examples of C₂₋₂₀ alkynyl groups include those related to C₂₋₂₀ alkyl groups by the insertion of one or more triple bonds. Alkynyl groups typically comprise one or two triple bonds. The alkynyl groups referred to herein may be substituted or unsubstituted, as defined for alkyl groups above.

The term “cycloalkyl group”, as used herein, refers to a substituted or unsubstituted alkyl group which is also a cyclyl group; that is, a monovalent moiety obtained by removing a hydrogen atom from an alicyclic ring atom of a carbocyclic ring of a carbocyclic compound. A cycloalkyl group may have from 3 to 25 carbon atoms (unless otherwise specified), including from 3 to 25 ring atoms. Thus, the term “cycloalkyl” includes the sub-classes cycloalkyenyl and cycloalkynyl.

Examples of groups of C₃₋₂₅ cycloalkyl groups include C₃₋₂₀ cycloalkyl, C₃₋₁₅ cycloalkyl, C₃₋₁₀ cycloalkyl, and C₃₋₇ cycloalkyl. When a C₃₋₂₅ cycloalkyl group is substituted it typically bears one or more substituents selected from substituted or unsubstituted C₁₋₆ alkyl, substituted or unsubstituted aryl, substituted or unsubstituted heteroaryl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted cycloalkyl group carries 1, 2 or 3 substituents, for instance 1 or 2.

Examples of C₃₋₂₅ cycloalkyl groups include, but are not limited to, those derived from saturated monocyclic hydrocarbon compounds, which C₃₋₂₅ cycloalkyl groups are substituted or unsubstituted as defined above: cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀); unsaturated monocyclic hydrocarbon compounds: cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈); saturated polycyclic hydrocarbon compounds: thujane (C₁₀), carane (C₁₀), pinane (C₁₀), bornane (C₁₀), norcarane (C₇), norpinane (C₇), norbomane (C₇), adamantane (C₁₀), decalin (decahydronaphthalene) (C₁₀); unsaturated polycyclic hydrocarbon compounds: camphene (C₁₀), limonene (C₁₀), pinene (C₁₀); polycyclic hydrocarbon compounds having an aromatic ring: indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

The term “heterocyclyl group”, as used herein, refers to a substituted or unsubstituted monovalent moiety obtained by removing a hydrogen atom from a ring atom of a heterocyclic compound, which moiety has from 3 to 20 ring atoms (unless otherwise specified), of which from 1 to 10 are ring heteroatoms. Heterocyclic compounds include aromatic heterocyclic compounds and non-aromatic heterocyclic compounds. Preferably, each ring has from 3 to 7 ring atoms, of which from 1 to 4 are ring heteroatoms. When a C₃₋₂₀ heterocyclyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₂₀ heterocyclyl group carries 1, 2 or 3 substituents, for instance 1 or 2.

Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of (non-aromatic) monocyclic C₃₋₂₀ heterocyclyl groups include, but are not limited to, those derived from:
N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of C₃₋₂₀ heterocyclyl groups which are also aryl groups are described below as heteroaryl groups.

The term “aryl group”, as used herein, refers to a substituted or unsubstituted, monocyclic or polycyclic (for instance bicyclic) aromatic group which typically contains from 6 to 14 carbon atoms, preferably from 6 to 10 carbon atoms in the ring portion. Examples include phenyl, naphthyl, indenyl, indanyl, anthracenyl and pyrenyl groups. An aryl group is substituted or unsubstituted. When an aryl group is substituted it typically bears one or more substituents selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Typically it carries 0, 1, 2 or 3 substituents. A substituted aryl group may be substituted in two positions with a single C₁₋₆ alkylene group, or with a bidentate group represented by the formula —X—C₁₋₆ alkylene, or —X—C₁₋₆ alkylene-X—, wherein X is selected from 0, S and NR, and wherein R is H, aryl or C₁₋₆ alkyl. Thus a substituted aryl group may be an aryl group fused with a cycloalkyl group or with a heterocyclyl group. The term “aralkyl” as used herein, pertains to an aryl group in which at least one hydrogen atom (e.g., 1, 2, 3) has been substituted with a C₁₋₆ alkyl group. Examples of such groups include, but are not limited to, tolyl (from toluene), xylyl (from xylene), mesityl (from mesitylene), and cumenyl (or cumyl, from cumene), and duryl (from durene).

The ring atoms of an aryl group may include one or more heteroatoms (as in a heteroaryl group). Such an aryl group is a heteroaryl group, and is a substituted or unsubstituted monocyclic or polycyclic (for instance bicyclic) heteroaromatic group which typically contains from 6 to 14 atoms, for instance 6 to 10 atoms, in the ring portion including one or more heteroatoms. It is generally a 5- or 6-membered ring, containing at least one heteroatom selected from O, S, N, P, Se and Si. It may contain, for example, 1, 2 or 3 heteroatoms. Examples of heteroaryl groups include pyridyl, pyrazinyl, pyrimidinyl, pyridazinyl, furanyl, benzofuranyl, dibenzofuranyl, thienyl, thiophenyl, benzothiophenyl, dibenzothiophenyl, pyrazolidinyl, pyrrolyl, oxazolyl, oxadiazolyl, isoxazolyl, thiadiazolyl, thiazolyl, isothiazolyl, imidazolyl, pyrazolyl, triazolyl, indolyl, benzimidazolyl, indazolyl, benzotriazolyl, pyrrolopyridinyl, pyrrolopyrimidinyl, purinyl, indolizinyl, pyrrolopyrazinyl, pyrrolopyriminyl, pyrrolopyridazinyl, imidazopyridinyl, pyrazolopyridinyl, imidazopyridazinyl, imidazopyrimidinyl, imidazopyrazinyl, imidazopyrimidinyl, triazolopyridinyl, quinolyl, isoquinolyl, cinnolinyl, quinazolinyl, quinoxalinyl, phthalazinyl, pyridopyrazinyl, pteridinyl, pyridopyridazinyl, naphthyridinyl, xanthanyl and carbazolyl. A heteroaryl group may be substituted or unsubstituted, for instance, as specified above for aryl. Typically it carries 0, 1, 2 or 3 substituents.

The term “benzyl group” or “benzylic group” refers the moiety —CH₂C₆H₅. Any of the hydrogens on the benzyl group may be substituted for a substituent as described herein. Typically, the benzyl group carries 0, 1, 2 or 3 substituents.

The term “alkylene group” as used herein, refers to an substituted or unsubstituted bidentate moiety obtained by removing two hydrogen atoms, either both from the same carbon atom, or one from each of two different carbon atoms, of a hydrocarbon compound having from 1 to 20 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below. Typically it is C₁₋₁₀ alkylene, for instance C₁₋₆ alkylene. Typically it is C₁₋₄ alkylene, for example methylene, ethylene, i-propylene, n-propylene, t-butylene, s-butylene or n-butylene. It may also be pentylene, hexylene, heptylene, octylene and the various branched chain isomers thereof. An alkylene group may be substituted or unsubstituted, for instance, as specified above for alkyl. Typically a substituted alkylene group carries 1, 2 or 3 substituents, for instance 1 or 2.

In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkylene,” as used herein, pertains to an alkylene group having from 1 to 4 carbon atoms. Examples of groups of alkylene groups include C₁₋₄ alkylene (“lower alkylene”), C₁₋₇ alkylene, C₁₋₁₀ alkylene and C₁₋₂₀ alkylene.

Examples of linear saturated C₁₋₇ alkylene groups include, but are not limited to, —(CH₂)ₙ— where n is an integer from 1 to 7, for example, —CH₂— (methylene), —CH₂CH₂— (ethylene), —CH₂CH₂CH₂-(propylene), and —CH₂CH₂CH₂CH₂— (butylene).

Examples of branched saturated C₁₋₇ alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₁₋₇ alkylene groups include, but are not limited to, —CH═CH— (vinylene), —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—.

Examples of branched partially unsaturated C₁₋₇ alkylene groups include, but are not limited to, —C(CH₃)=CH—, —C(CH₃)=CH—CH₂—, and —CH═CH—CH(CH₃)—.

Partially unsaturated alkylene groups comprising one or more double bonds may be referred to as alkenylene groups. Partially unsaturated alkylene groups comprising one or more triple bonds may be referred to as alkynylene groups (for instance —C≡C—, CH₂—C≡C—, and —CH₂—C≡C—CH₂—).

Examples of alicyclic saturated C₁₋₇ alkylene groups include, but are not limited to, cyclopentylene (e.g., cyclopent-1,3-ylene), and cyclohexylene (e.g., cyclohex-1,4-ylene).

Examples of alicyclic partially unsaturated C₁₋₇ alkylene groups include, but are not limited to, cyclopentenylene (e.g., 4-cyclopenten-1,3-ylene), cyclohexenylene (e.g., 2-cyclohexen-1,4-ylene; 3-cyclohexen-1,2-ylene; 2,5-cyclohexadien-1,4-ylene).

As used herein the term “oxo” represents a group of formula: ═O.

As used herein the term “acyl” represents a group of formula: —C(═O)R, wherein R is an acyl substituent, for example, a substituted or unsubstituted C₁₋₂₀ alkyl group, substituted or unsubstituted C₂₋₂₀ alkenyl group, substituted or unsubstituted C₂₋₂₀ alkynyl group, a substituted or unsubstituted C₃₋₂₀ heterocyclyl group, a substituted or unsubstituted aryl group or a substituted or unsubstituted heteroaryl group, for instance a substituted or unsubstituted C₁₋₆ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

As used herein the term “acyloxy” (or reverse ester) represents a group of formula: —OC(═O)R, wherein R is an acyloxy substituent, for example, a substituted or unsubstituted C₁₋₂₀ alkyl group, substituted or unsubstituted C₂₋₂₀ alkenyl group, substituted or unsubstituted C₂₋₂₀ alkynyl group, a substituted or unsubstituted C₃₋₂₀ heterocyclyl group, a substituted or unsubstituted aryl group or a substituted or unsubstituted heteroaryl group, for instance a substituted or unsubstituted C₁₋₆ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

As used herein the term “ester” (or carboxylate, carboxylic acid ester or oxycarbonyl) represents a group of formula: —C(═O)OR, wherein R is an ester substituent, for example, a substituted or unsubstituted C₁₋₂₀ alkyl group, substituted or unsubstituted C₂₋₂₀ alkenyl group, substituted or unsubstituted C₂₋₂₀ alkynyl group, a substituted or unsubstituted C₃₋₂₀ heterocyclyl group, a substituted or unsubstituted aryl group or a substituted or unsubstituted heteroaryl group, for instance a substituted or unsubstituted C₁₋₆ alkyl group. Examples of ester groups include, but are not limited to, —C(═O)OCH₃, —C(═O)OCH₂CH₃, —C(═O)OC(CH₃)₃, and —C(═O)OPh.

As used herein the term “amino” represents a group of formula —NH₂. The term “C₁-C₁₀ alkylamino” represents a group of formula —NHR′ wherein R′ is a C₁₋₁₀ alkyl group, preferably a C₁₋₆ alkyl group, as defined previously. The term “di(C₁₋₁₀)alkylamino” represents a group of formula —NR′R″ wherein R′ and R″ are the same or different and represent C₁₋₁₀ alkyl groups, preferably C₁₋₆ alkyl groups, as defined previously. The term “arylamino” represents a group of formula —NHR′ wherein R′ is an aryl group, preferably a phenyl group, as defined previously. The term “diarylamino” represents a group of formula —NR′R″ wherein R′ and R″ are the same or different and represent aryl groups, preferably phenyl groups, as defined previously. The term “arylalkylamino” represents a group of formula —NR′R″ wherein R′ is a C₁₋₁₀ alkyl group, preferably a C₁₋₆ alkyl group, and R″ is an aryl group, preferably a phenyl group.

A halo group is chlorine, fluorine, bromine or iodine (a chloro group, a fluoro group, a bromo group or an iodo group). It is typically chlorine, fluorine or bromine.

The term “halide”, as used herein, refers to fluoride, chloride, bromide and iodide.

As used herein the term “amido” represents a group of formula: —C(═O)NR′R″, wherein R′ and R″ are independently amino substituents, as defined for di(C₁₋₁₀)alkylamino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′ and R″, together with the nitrogen atom to which they are attached, form a heterocyclic structure as in, for example, piperidinocarbonyl, morpholinocarbonyl, thiomorpholinocarbonyl, and piperazinocarbonyl.

As used herein the term “acylamido” represents a group of formula: —NR′C(═O)R″, wherein R′ is an amide substituent, for example, hydrogen, a C₁₋₂₀ alkyl group, a C₃₋₂₀ heterocyclyl group, an aryl group, preferably hydrogen or a C₁₋₂₀ alkyl group, and R″ is an acyl substituent, for example, a C₁₋₂₀ alkyl group, a C₃₋₂₀ heterocyclyl group, or an aryl group, preferably hydrogen or a C₁₋₂₀ alkyl group. Examples of acylamide groups include, but are not limited to, —NHC(═O)CH₃, —NHC(═O)CH₂CH₃, —NHC(═O)Ph, —NHC(═O)C₁₅H₃₁ and —NHC(═O)C₉H₁₉. Thus, a substituted C₁₋₂₀ alkyl group may comprise an acylamido substituent defined by the formula —NHC(═O)—C₁₋₂₀ alkyl, such as —NHC(═O)C₁₅H₃₁ or —NHC(═O)C₉H₁₉. R¹ and R² may together form a cyclic structure, as in, for example, succinimidyl, maleimidyl, and phthalimidyl:

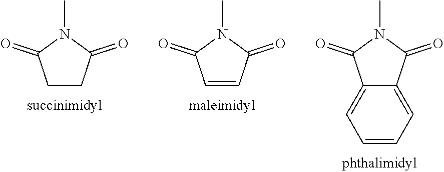

A C₁₋₁₀ alkylthio group is a said C₁₋₁₀ alkyl group, preferably a C₁₋₆ alkyl group, attached to a thio group. An arylthio group is an aryl group, preferably a phenyl group, attached to a thio group.

A C₁₋₂₀ alkoxy group is a said substituted or unsubstituted C₁₋₂₀ alkyl group attached to an oxygen atom. A C₁₋₆ alkoxy group is a said substituted or unsubstituted C₁₋₆ alkyl group attached to an oxygen atom. A C₁₋₄ alkoxy group is a substituted or unsubstituted C₁₋₄ alkyl group attached to an oxygen atom. Examples of C₁₋₄ alkoxy groups include, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy). Further examples of C₁₋₂₀ alkoxy groups are —O(Adamantyl), —O—CH₂-Adamantyl and —O—CH₂—CH₂-Adamantyl.

An aryloxy group is a substituted or unsubstituted aryl group, as defined herein, attached to an oxygen atom. An example of an aryloxy group is —OPh (phenoxy).

Unless otherwise specified, included in the above are the well-known ionic, salt, solvate, and protected forms of these substituents. For example, a reference to carboxylic acid or carboxyl group (—COOH) also includes the anionic (carboxylate) form (—COO⁻), a salt or solvate thereof, as well as conventional protected forms. Similarly, a reference to an amino group includes the protonated form (—N⁺HR′R″), a salt or solvate of the amino group, for example, a hydrochloride salt, as well as conventional protected forms of an amino group. Similarly, a reference to a hydroxyl group also includes the anionic form (—O—), a salt or solvate thereof, as well as conventional protected forms.

Certain compounds may exist in one or more particular geometric, optical, enantiomeric, diastereomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto, enol, and enolate forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting known methods, in a known manner.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like, unless otherwise specified. However, reference to an isotope of fluorine refers only to that isotope of fluorine. In particular, reference to ¹⁸F includes only ¹⁸F. Reference to fluorine without specifying the isotope may refer to ¹⁸F or ¹⁹F depending on context. Typically, reference to “F” (i.e. without defining the isotope) refers to the ¹⁹F, i.e. stable fluorine.

The term “substituted”, as used herein, may be as defined above for particular groups. However, in some instances, the term substituted may refer to a group substituted with a group selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. In other instances, the term “substituted” may refer to a group substituted with a group selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted cycloalkyl, substituted or unsubstituted heterocyclyl, cyano, amino, C₁₋₆ alkylamino, di(C₁₋₆)alkylamino, arylamino, diarylamino, arylalkylamino, amido, acylamido, hydroxy, oxo, halo, carboxy, ester, acyl, acyloxy, C₁₋₆ alkoxy, aryloxy, haloalkyl, sulfonic acid, thiol, C₁₋₆ alkylthio, arylthio, sulfonyl, phosphoric acid, phosphate ester, phosphonic acid and phosphonate ester. For example, the term “substituted” may refer to a group substituted with a group selected unsubstituted C₁₋₆ alkyl, unsubstituted C₂₋₆ alkenyl, unsubstituted C₂₋₆ alkynyl, unsubstituted aryl, unsubstituted heteroaryl, cyano, amino, unsubstituted C₁₋₆ alkylamino, unsubstituted di(C₁₋₆)alkylamino, unsubstituted arylamino, unsubstituted diarylamino, unsubstituted arylalkylamino, unsubstituted amido, unsubstituted acylamido, hydroxy, oxo, halo, carboxy, unsubstituted ester, unsubstituted acyl, unsubstituted acyloxy, unsubstituted C₁₋₆ alkoxy, unsubstituted aryloxy, sulfonic acid, thiol, unsubstituted C₁₋₆ alkylthio, unsubstituted arylthio, sulfonyl, phosphoric acid, unsubstituted phosphate ester, unsubstituted phosphonic acid and unsubstituted phosphonate ester.

The term “¹⁸F” refers to an atom of the specific isotope of fluorine having 9 protons and 9 neutrons. The terms “¹⁸F⁻” and “¹⁸F-fluoride” refer to an anion of the atom of the specific isotope of fluorine having 9 protons and 9 neutrons.

The use of “¹⁸F⁻” before a chemical entity name or “¹⁸F[chemical formula]” refers to a chemical entity in which a ¹⁹F has been replaced with an ¹⁸F. Therefore, the terms “¹⁸F-trifluoromethyl” and “¹⁸F[CF₃]” as used herein refer to a —CF₃ (trifluoromethyl) group in which one of the three fluorines is ¹⁸F, i.e. a group of formula —CF₂¹⁸F.

The term “carbene” as used herein refers to electrically neutral species H₂C: and its derivatives, in which the carbon is covalently bonded to two univalent groups of any kind or a divalent group. The carbon of the carbene bears two nonbonding electrons, which may be spin-paired (singlet state) or spin-non-paired (triplet state). The term “difluorocarbene” as used herein refers to F₂C:.

The term “ligand”, as used herein, refers to a species capable of binding to a central atom to form a complex. Ligands may be charged or neutral species. Typically, as referred to herein, a ligand is a neutral species.

### Process

The invention provides a process for producing a compound comprising an ¹⁸F-difluoromethyl group or a compound comprising an ¹⁸F-difluoromethylene group, which process comprises contacting a compound comprising a nucleophilic group with ¹⁸F-difluorocarbene.

An ¹⁸F-difluoromethyl group is a group —CHF¹⁸F. In some instances, (for example when the reaction is carried out in the presence of deuterated solvent) the ¹⁸F-difluoromethyl group may be deuterated (i.e. —CDF¹⁸F). Throughout this specification the term “¹⁸F-difluoromethyl group” refers to both the deuterated and non-deuterated variants. An ¹⁸F-difluoromethylene group is a divalent group —CF¹⁸F—.

Typically, contacting the compound with ¹⁸F-difluorcarbene comprises treating the compound comprising a nucleophilic group with a source of ¹⁸F-difluorocarbene. The source of ¹⁸F-difluorocarbene is typically a reagent capable of generating ¹⁸F-difluorocarbene. Typically, the reagent is capable of generating ¹⁸F-difluorocarbene in situ i.e. under the same reaction conditions used when the compound comprising a nucleophilic group is contacted with ¹⁸F-difluorocarbene.

Typically, the nucleophilic group undergoes a reaction to produce the compound comprising the ¹⁸F-difluoromethyl group or the compound comprising the ¹⁸F-difluoromethylene group. Thus, the nucleophilic group of the compound comprising a nucleophilic group typically undergoes a chemical transformation to a different chemical moiety during the reaction process. The ¹⁸F-difluorocarbene may for example insert into a chemical bond. For instance, the ¹⁸F-difluorocarbene may insert into an X—H bond to form the group —XCF¹⁸FH, where X may be S, N or O. The ¹⁸F-difluorocarbene may react with an alkene to form a ¹⁸F-difluorocyclopropane group.

The process typically comprises treating a compound comprising a nucleophilic group with a compound of formula (I):

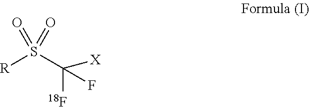

wherein X is H or Cl, preferably wherein X is H, and wherein R is a substituted or unsubstituted aromatic group. Typically, the process comprises treating a compound comprising a nucleophilic group with a compound of formula (I) in the presence of a base. Typically, the process comprises treating a compound comprising a nucleophilic group with a compound of formula (I) in which X is H in the presence of a base.

Thus, the source of ¹⁸F-difluorocarbene is usually a compound of formula (I) as described herein. Thus, the nucleophilic group typically reacts with the CF¹⁸F moiety from the compound of formula (I) to produce the compound comprising the ¹⁸F-difluoromethyl group or the compound comprising the ¹⁸F-difluoromethylene group.

In the reaction the compound of formula (I) usually undergoes a reaction to generate ¹⁸F-difluorocarbene in situ. The compound of formula (I) typically provides ¹⁸F-difluorocarbene via an alpha elimination reaction. For instance, when a compound of formula (I), where X is H, is treated with a base, deprotonation occurs. The deprotonated molecule then undergoes alpha elimination to liberate ¹⁸F-difluorocarbene. Alpha elimination reaction is a transformation of the type:

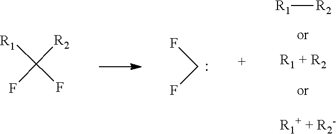

wherein R₁ and R₂ are leaving groups. The loss of R₁ and R₂ from the central CF₂ group generates difluorocarbene (:CF₂). Typically, the difluorocarbene generated is free difluorocarbene.

In the compound of formula (I), R may be selected from substituted or unsubstituted aryl and substituted or unsubstituted heteroaryl. For instance, R may be a substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl. Preferably, R is substituted or unsubstituted phenyl.

Alternatively, R may be a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted benzothiazolyl and substituted or unsubstituted carbazolyl.

Preferably R is substituted or unsubstituted pyridyl, substituted or unsubstituted pyrimidyl or substituted or unsubstituted benzothiazolyl.

R may be substituted with one or more substituents, as described herein. In some instances, R is substituted with one or more electron withdrawing substituents. In some instances, R is substituted with one or more electron donating substituents. Typically the one or more substituents are different from the nucleophilic group on the compound comprising a nucleophilic group.

Typically, R is substituted with 1, 2 or 3 substituents as described herein. Typically, R is substituted with 1, 2 or 3 substituents selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted cycloalkyl, substituted or unsubstituted heterocyclyl, cyano, amino, C₁₋₆ alkylamino, di(C₁₋₆)alkylamino, arylamino, diarylamino, arylalkylamino, amido, acylamido, nitro, hydroxy, oxo, halo, carboxy, ester, acyl, acyloxy, C₁₋₆ alkoxy, aryloxy, haloalkyl, sulfonic acid, thiol, C₁₋₆ alkylthio, arylthio, sulfonyl, phosphoric acid, phosphate ester, phosphonic acid and phosphonate ester. For instance, the aromatic group may be substituted with 1, 2 or 3 substituents selected from the group consisting of unsubstituted C₁₋₆ alkyl, unsubstituted C₂₋₆ alkenyl, unsubstituted C₂₋₆ alkynyl, unsubstituted aryl, unsubstituted heteroaryl, cyano, amino, unsubstituted C₁₋₆ alkylamino, unsubstituted di(C₁₋₆)alkylamino, unsubstituted arylamino, unsubstituted diarylamino, unsubstituted arylalkylamino, unsubstituted amido, unsubstituted acylamido, hydroxy, oxo, halo, carboxy, unsubstituted ester, unsubstituted acyl, unsubstituted acyloxy, unsubstituted C₁₋₆ alkoxy, unsubstituted aryloxy, sulfonic acid, thiol, unsubstituted C₁₋₆ alkylthio, unsubstituted arylthio, sulfonyl, phosphoric acid, unsubstituted phosphate ester, unsubstituted phosphonic acid and unsubstituted phosphonate ester. Typically, R is substituted with 1, 2 or 3 substituents which are unsubstituted C₁₋₆ alkyl, nitro or halo. For instance, R may be substituted with 1, 2 or 3 substituents selected from methyl, ethyl, propyl, butyl, pentyl, hexyl. Preferably R is substituted with ^{t}butyl. Preferably R is substituted with para-^{t}butyl.

R may be substituted with 1, 2 or 3 substituents which comprise a nitro group. For instance, R may be substituted with 1 substituent which is a nitro group. Preferably R is substituted with a para-nitro group.

R may be substituted with 1, 2 or 3 substituents which are halogens. For instance, R may be substituted with 1, 2 or 3 substituents selected from the group consisting of F, Cl, Br and I. Typically, R is substituted with 1, 2 or 3 substituents which comprise Cl. Preferably R is substituted with 1 substituent which is Cl, more preferably R is substituted with para-Cl.

Typically, the compound of formula (I) is a compound having the following formula:

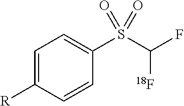

wherein R is selected from the group consisting of Bu, nitro and Cl, preferably wherein R is Cl.

Typically, the compound of formula (I) is a compound having the following formula:

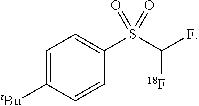 [SMILES: CC(C)(C)c1ccc(S(=O)(=O)C(F)[18F])cc1]

The ¹⁸F-difluorocarbene may be generated by treating the compound of formula (I) with a base. For instance, the ¹⁸F-difluorocarbene may be generated by treating a compound having the following formula:

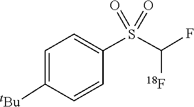 [SMILES: CC(C)(C)c1ccc(S(=O)(=O)C(F)[18F])cc1]

with abase.

Preferably, the compound of formula (I) is a compound having the following formula:

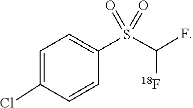 [SMILES: O=S(=O)(c1ccc(Cl)cc1)C(F)[18F]]

The ¹⁸F-difluorocarbene may be generated by treating the compound of formula (I) with a base. For instance, the ¹⁸F-difluorocarbene may be generated by treating a compound having the following formula:

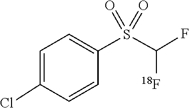 [SMILES: O=S(=O)(c1ccc(Cl)cc1)C(F)[18F]]

with a base.

The base may be any base suitable to generate the ¹⁸F-difluorocarbene and is preferably also compatible with the compound comprising a nucleophilic group. In some instances, the compound comprising a nucleophilic group may itself act as a base, such that no additional base is required. In other instances, the base may be an additional base, which may be any suitable base known to the skilled person. The skilled person would be well aware of suitable bases for deprotonating the compound of formula (I) and would be able to choose a base appropriate for the compound comprising a nucleophilic group. Typically, the base is selected from metal hydroxides and metal hydrides. Typically the base is selected from alkali metal hydroxides and alkali metal hydrides. For instance, the base may be selected from NaOH, KOH and NaH.

The base is typically dissolved in a solvent. Typically the solvent is an aqueous solvent i.e. a solvent which comprises or consists of water. In some instances, the reagents may not be compatible with water in which case the base may be dissolved in a non-aqueous solvent. Typically, the non-aqueous solvent is an organic solvent, preferably a polar organic solvent. For instance, the base may be dissolved in a non-aqueous solvent such as dimethylformamide (DMF) or acetonitrile (MeCN).

### Compound Comprising a Nucleophilic Group

The nucleophilic group of the compound comprising a nucleophilic group typically comprises an atom or ion with a lone pair of electrons. The nucleophilic group may comprise a π bond.

For instance, the nucleophilic group may be —XH, where X is a heteroatom, or the nucleophilic group may be selected from an alkene, an alkyne, a phosphonate, a sulfinate, an aldehyde and a functional group disposed to tautomerism The heteroatom may be selected from O, S, N, P, Se and Si, preferably from O, S, N and P. Typically, the nucleophilic group is selected from the group consisting of —OH, —SH or —NH. The skilled person would be well aware of which functional groups are disposed to tautomerisation and their ability to act as nucleophiles. Groups disposed to tautomerism include ketones, amides, imines, enamines, nitro groups, nitroso groups, ketenes and phosphonates.

Typically, when the nucleophilic group is NH, the NH group is comprised in an N-heterocyclic or N-heteroaromatic ring. Thus, when the nucleophilic group is NH and the compound comprising a nucleophilic group may comprise an N-heterocyclic ring wherein the nitrogen atom of Nuc is a ring atom in the heterocyclic ring

The nucleophilic group may be generated in situ. For example, where the nucleophilic group is an OH group, the OH group may be generated from a boronic acid or boronic ester precursor through reaction with an oxidising agent such as hydrogen peroxide. These reactions may be conducted in a “one-pot” fashion, with reaction of the boronic acid precursor to provide the OH group followed by reaction with a compound of formula (I). This process may be extended to apply to other starting materials, for instance aryl or benzyl halides. Thus, the compound comprising a nucleophilic group may be formed in situ from an aromatic group or benzylic group, as described herein, comprising a halo substituent, for instance chloride. The halo substituent is reacted to form a boronic acid or boronic ester which is subsequently treated with an oxidising agent to provide the nucleophilic group, which then goes on to react with the ¹⁸F-difluorocarbene. This process may be performed in a “one-pot” fashion. An example of this process is provided in Example 4.

Where the nucleophilic group is an alkyne, deprotonation of the alkyne may occur to provide the nucleophilic anion.

Typically, the compound comprising the nucleophilic group comprises an substituted or unsubstituted aromatic group or a substituted or unsubstituted benzylic group. The aromatic group may be selected from substituted or unsubstituted aryl and substituted or unsubstituted heteroaryl. The term “substituted” is as described herein. Typically, the compound comprising a nucleophilic group comprises a substituted aromatic group or a substituted benzylic group bearing 1, 2 or 3 substituents as described herein. Typically, the compound comprising a nucleophilic group comprises an aromatic group selected from the group consisting of an unsubstituted aryl group, an unsubstituted heteroaryl group, an aryl group substituted aromatic group bearing 1, 2 or 3 substituents as described herein or a heteroaryl group bearing 1, 2 or 3 substituents as described herein. The compound comprising a nucleophilic group may comprise an benzylic group selected from the group consisting of an unsubstituted benzylic group or a substituted benzylic group bearing 1, 2 or 3 substituents as described herein.

For the avoidance of doubt, if the compound comprising a nucleophilic group comprises a nucleophilic group (for instance an OH group) bound directly to the benzyl, aryl or heteroaryl group, then the nucleophilic group itself is not counted as one of the substituents. In other words, the substituents are additional to the nucleophilic group.

Typically, the aromatic group or benzylic group is substituted with 1, 2 or 3 substituents selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted cycloalkyl, substituted or unsubstituted heterocyclyl, cyano, amino, C₁₋₆ alkylamino, di(C₁₋₆)alkylamino, arylamino, diarylamino, arylalkylamino, amido, acylamido, hydroxy, oxo, halo, carboxy, ester, acyl, acyloxy, C₁₋₆ alkoxy, aryloxy, haloalkyl, sulfonic acid, thiol, C₁₋₆ alkylthio, arylthio, sulfonyl, phosphoric acid, phosphate ester, phosphonic acid and phosphonate ester. For instance, the aromatic group may be substituted with 1, 2 or 3 substituents selected from the group consisting of unsubstituted C₁₋₆ alkyl, unsubstituted C₂₋₆ alkenyl, unsubstituted C₂₋₆ alkynyl, unsubstituted aryl, unsubstituted heteroaryl, cyano, amino, unsubstituted C₁₋₆ alkylamino, unsubstituted di(C₁₋₆)alkylamino, unsubstituted arylamino, unsubstituted diarylamino, unsubstituted arylalkylamino, unsubstituted amido, unsubstituted acylamido, hydroxy, oxo, halo, carboxy, unsubstituted ester, unsubstituted acyl, unsubstituted acyloxy, unsubstituted C₁₋₆ alkoxy, unsubstituted aryloxy, sulfonic acid, thiol, unsubstituted C₁₋₆ alkylthio, unsubstituted arylthio, sulfonyl, phosphoric acid, unsubstituted phosphate ester, unsubstituted phosphonic acid and unsubstituted phosphonate ester.

The compound comprising the nucleophilic group may comprise a substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl. Preferably the compound comprising the nucleophilic group comprises a substituted or unsubstituted phenyl group. The substituents on the aryl group may be 1, 2 or 3 substituents selected from those listed above for thearomatic group.

The compound comprising the nucleophilic group may comprise a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted thiophenyl, substituted or unsubstituted benzofuranyl, substituted or unsubstituted dibenzofuranyl, substituted or unsubstituted benzothiphenyl, substituted or unsubstituted dibenzothiophenyl, substituted or unsubstituted xanthenyl and substituted or unsubstituted carbazolyl. Preferably, the compound comprising the nucleophilic group comprises a substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl and substituted or unsubstituted quinolinyl group. The substituents on the heteroaryl group may be 1, 2 or 3 substituents selected from those listed above for the aromatic group.

Typically, the nucleophilic group is bonded to the aromatic group. For instance, the nucleophilic group may be directly bonded to the aromatic group or may be bonded through a linking group. Hence, the compound comprising a nucleophilic group may be a compound of formula (II):

Ar-L-Nuc Formula (II)

where Ar represents a substituted or unsubstituted aromatic group, wherein the aromatic group is as described herein, L represents a linking group or a bond, and wherein Nuc represents a nucleophilic group as described herein.

For instance, Ar may represent a substituted or unsubstituted aryl group as described herein, or a substituted or unsubstituted heteroaryl group as described herein. Ar may represent a substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl. Preferably, Ar represents a substituted or unsubstituted phenyl group. The substituents on the aryl group may be 1, 2 or 3 substituents selected from those listed above for the aromatic group.

Ar may represent a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted thiophenyl, substituted or unsubstituted benzofuranyl, substituted or unsubstituted dibenzofuranyl, substituted or unsubstituted benzothiphenyl, substituted or unsubstituted dibenzothiophenyl, substituted or unsubstituted xanthenyl and substituted or unsubstituted carbazolyl. Preferably, Ar represents a substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl or substituted or unsubstituted quinolinyl group. The substituents on the heteroaryl group may be 1, 2 or 3 substituents selected from those listed above for the aromatic group.

L is typically selected from a bond or a substituted or unsubstituted C₁₋₁₀ alkylene. For instance, L may be selected from a bond or an unsubstituted C₁₋₄ alkylene. Preferably, L is a bond.

Nuc typically represents an atom or ion with a lone pair of electrons. The nucleophilic group may comprise a π bond. For instance, Nuc may be —XH, where X is a heteroatom, or the nucleophilic group may be selected from an alkene, an alkyne, a phosphonate, a sulfinate, an aldehyde and a functional group disposed to tautomerism. The heteroatom may be selected from O, S, N, P, Se and Si, preferably from O, S, N and P. Typically, Nuc is selected from the group consisting of —OH, —SH or —NH. For instance, Nuc may represent an OH or SH group.

When Nuc is NH and the compound comprising a nucleophilic group may comprise an N-heterocyclic ring wherein the nitrogen atom of Nuc is a ring atom in the heterocyclic ring Typically, the N-heterocyclic ring is in a N-containing heteroaryl. For instance, the N atom may be comprised in a substituted or unsubstituted pyridyl, substituted or unsubstituted pyrazinyl, substituted or unsubstituted pyrimidinyl, substituted or unsubstituted pyridazinyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, or a substituted or unsubstituted carbazolyl group. Preferably, the N atom is comprised in a substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl or a substituted or unsubstituted quinolinyl group. The substituents on the N-heterocyclic ring may be 1, 2 or 3 substituents selected from those listed above for the aromatic group.

For instance, the compound comprising a nucleophilic group may be selected from:

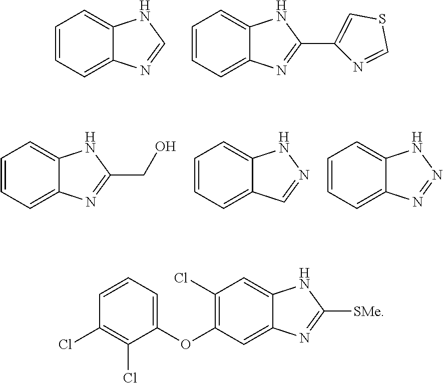

The nucleophilic group in the compound comprising a nucleophilic group may be an alkene group. For instance, the compound comprising a nucleophilic group may be a compound having the following formula:

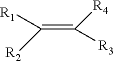

wherein R₁, R₂, R₃ and R₄ are each independently selected from the group consisting of hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Typically, R₁, R₂, R₃ and R₄ are each independently selected from the group consisting of hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl and substituted or unsubstituted heterocyclyl. For instance, R₁, R₂, R₃ and R₄ may each independently selected from the group consisting of unsubstituted C₁₋₆ alkyl, unsubstituted C₂₋₆ alkenyl, unsubstituted C₂₋₆ alkynyl, unsubstituted aryl and unsubstituted heteroaryl.

The compound comprising a nucleophilic group may be

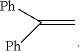

The nucleophilic group in the compound comprising a nucleophilic group may be an alkyne functional group. The compound comprising a nucleophilic group may therefore be a compound having the following formula:

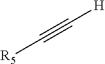

where R₅ is selected from the group consisting of hydrogent, substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Typically, R₅ is selected from the group consisting of hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl and substituted or unsubstituted heterocyclyl.

The nucleophilic group in the compound comprising a nucleophilic group may be an OH group. Typically, the O atom in the OH group is bonded directly to an aromatic group. For instance, the O atom may be bonded directly to substituted or unsubstituted aryl group as described herein, or a substituted or unsubstituted heteroaryl group as described herein.

The O atom may be bonded directly to substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl. Preferably, the O atom is bonded directly to a substituted or unsubstituted phenyl group. The substituents on the aryl group may be 1, 2 or 3 substituents selected from those listed above for the aromatic group.

The O atom may be bonded directly to a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted thiophenyl, substituted or unsubstituted benzofuranyl, substituted or unsubstituted dibenzofuranyl, substituted or unsubstituted benzothiphenyl, substituted or unsubstituted dibenzothiophenyl, substituted or unsubstituted xanthenyl and substituted or unsubstituted carbazolyl. Typically, the O atom is bonded directly to a substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl or substituted or unsubstituted quinolinyl group. The substituents on the heteroaryl group may be 1, 2 or 3 substituents selected from those listed above for the aromatic group.

For instance, the compound comprising a nucleophilic group may be selected from:

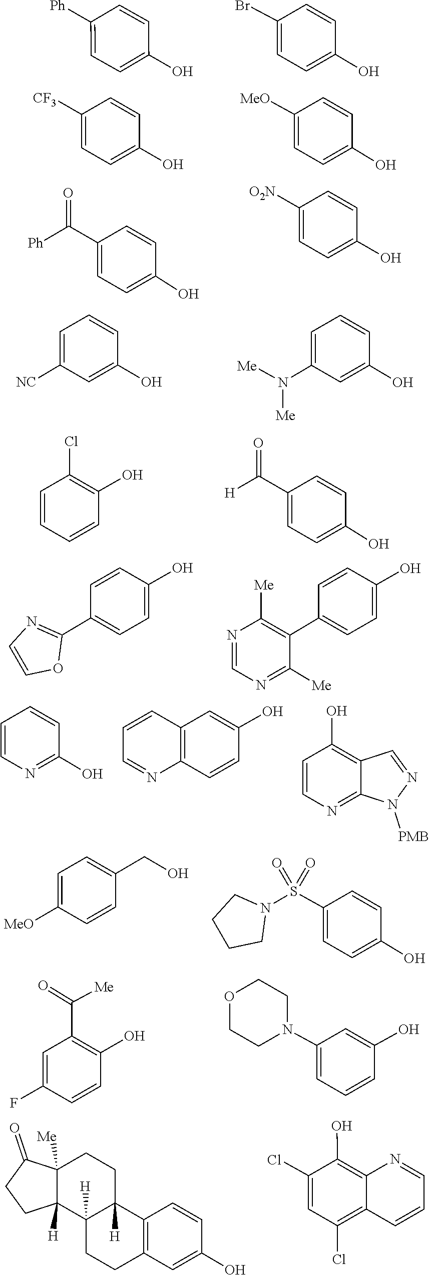

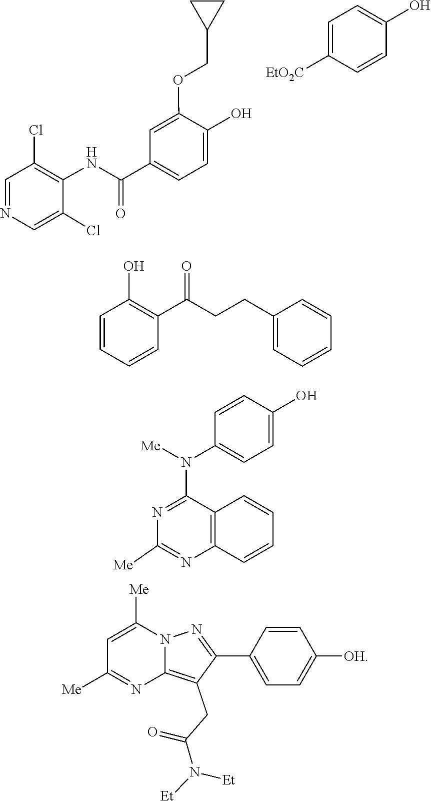

The nucleophilic group in the compound comprising a nucleophilic group may be an —SH functional group. The S atom may be bonded directly to substituted or unsubstituted aryl group as described herein, or a substituted or unsubstituted heteroaryl group as described herein.

The S atom may be bonded directly to substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl. Preferably, Ar represents a substituted or unsubstituted phenyl group. The substituents on the aryl group may be 1, 2 or 3 substituents selected from those listed above for the aromatic group.

The S atom may be bonded directly to a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted thiophenyl, substituted or unsubstituted benzofuranyl, substituted or unsubstituted dibenzofuranyl, substituted or unsubstituted benzothiphenyl, substituted or unsubstituted dibenzothiophenyl, substituted or unsubstituted xanthenyl and substituted or unsubstituted carbazolyl. The S atom may be bonded directly to a substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl or substituted or unsubstituted quinolinyl group. The substituents on the heteroaryl group may be 1, 2 or 3 substituents selected from those listed above for the aromatic group.

The compound comprising a nucleophilic group may be selected from the following compounds:

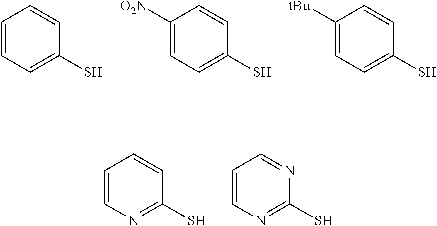

The compound comprising a ¹⁸F-difluoromethyl group or the compound comprising an ¹⁸F-difluoromethylene group may be a positron emission tomography (PET) ligand.

The compound comprising a ¹⁸F-difluoromethyl group may be an ¹⁸F-difluoromethyl functionalised microtubule ligand, an ¹⁸F-difluoromethyl functionalised microtubule ligand PK-M2 ligand, an ¹⁸F-difluoromethyl functionalised microtubule ligand P2X₇R ligand, an ¹⁸F-difluoromethyl functionalised microtubule ligand mGluR2 ligand, an ¹⁸F-difluoromethyl functionalised TSPO ligand or an ¹⁸F-difluoromethyl functionalised GABAA receptor antagonist.

### Other Process Features

Typically, treating the compound comprising a nucleophilic group is performed in the presence of a solvent. The solvent may be a single solvent or a mixture of solvents. For instance, the solvent may comprise or consist of an organic solvent. The solvent may comprise or consist of water i.e. the solvent may be an aqueous solvent. In cases where the reagents are sensitive to water, the solvent will comprise no or very small amounts (less than 0.5% by weight, typically less than 0.1% by weight) water. The solvent may comprise or consist of a polar aprotic solvent. For instance, the solvent may comprise or consist of acetonitrile, propylene carbonate, DMSO or DMF.

In some instances the solvent may be a deuterated solvent. Deuterated solvents may be used when the incorporation of D into the final [¹⁸F]difluoromethyl or [¹⁸F]difluoromethylene-containing product is desired.

Typically, treating the compound comprising a nucleophilic group is performed at a temperature above room temperature (i.e above 25° C.). For instance, treating the compound comprising a nucleophilic group may be performed at a temperature of at least 40° C. Treating the compound comprising a nucleophilic group may be performed at a temperature of no more than 300° C., no more than 250° C., no more than 200° C., no more than 150° C. or no more than 100° C. Preferably, treating the compound comprising a nucleophilic group is performed at a temperature of between 50° C. and 250° C. For instance, treating the compound comprising a nucleophilic group is performed at a temperature of between 50° C. and 200° C., or a temperature of between 50° C. and 150° C., or a temperature of between 50° C. and 100° C. The treatment step may last at least 1 minute, for instance at least 5 minutes or at least 10 minutes. Typically, the treatment step lasts no longer than one hour, typically no longer than 45 minutes, preferably no longer than 30 minutes. Thus, the treatment step usually lasts from 1 minute to 1 hour, for instance from 5 minutes to 45 minutes, preferably from 10 minutes to 30 minutes.

### DFC Reagent

The invention also provides a compound of formula (I):

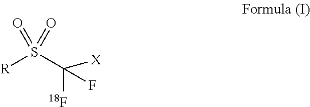

where X is H or Cl, and wherein R is a substituted or unsubstituted aryl group. Preferably X is H.

R may be a substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl. Preferably, R is substituted or unsubstituted phenyl.

R may be substituted with one or more substituents, as described herein. In some instances, R is substituted with one or more electron withdrawing substituents. In some instances, R is substituted with one or more electron donating substituents.

Typically, R is substituted with 1, 2 or 3 substituents as described herein. Typically, R is substituted with 1, 2 or 3 substituents selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted cycloalkyl, substituted or unsubstituted heterocyclyl, cyano, amino, C₁₋₆ alkylamino, di(C₁₋₆)alkylamino, arylamino, diarylamino, arylalkylamino, amido, acylamido, hydroxy, oxo, nitro, halo, carboxy, ester, acyl, acyloxy, C₁₋₆ alkoxy, aryloxy, haloalkyl, sulfonic acid, thiol, C₁₋₆ alkylthio, arylthio, sulfonyl, phosphoric acid, phosphate ester, phosphonic acid and phosphonate ester. For instance, the aromatic group may be substituted with 1, 2 or 3 substituents selected from the group consisting of unsubstituted C₁₋₆ alkyl, unsubstituted C₂₋₆ alkenyl, unsubstituted C₂₋₆ alkynyl, unsubstituted aryl, unsubstituted heteroaryl, cyano, amino, unsubstituted C₁₋₆ alkylamino, unsubstituted di(C₁₋₆)alkylamino, unsubstituted arylamino, unsubstituted diarylamino, unsubstituted arylalkylamino, unsubstituted amido, unsubstituted acylamido, hydroxy, oxo, nitro, halo, carboxy, unsubstituted ester, unsubstituted acyl, unsubstituted acyloxy, unsubstituted C₁₋₆ alkoxy, unsubstituted aryloxy, sulfonic acid, thiol, unsubstituted C₁₋₆ alkylthio, unsubstituted arylthio, sulfonyl, phosphoric acid, unsubstituted phosphate ester, unsubstituted phosphonic acid and unsubstituted phosphonate ester. Typically, R is substituted with 1, 2 or 3 substituents selected from the group consisting of unsubstituted C₁₋₆ alkyl, nitro and halo. Typically, R is substituted with 1, 2 or 3 substituents which are unsubstituted C₁₋₆ alkyl. For instance, R may be substituted with 1, 2 or 3 substituents selected from methyl, ethyl, propyl, butyl, pentyl, hexyl. Preferably R is substituted with ^{t}butyl. Preferably R is substituted with para-^{t}butyl.

R may be substituted with 1, 2 or 3 substituents which comprise a nitro group. For instance, R may be substituted with 1 substituent which is a nitro group. Preferably R is substituted with a para-nitro group.

R may be substituted with 1, 2 or 3 substituents which are halogens. For instance, R may be substituted with 1, 2 or 3 substituents selected from the group consisting of F, Cl, Br and I. Typically, R is substituted with 1, 2 or 3 substituents which comprise Cl. Preferably R is substituted with 1 substituent which is Cl, more preferably R is substituted with para-Cl.

Typically, the compound of formula (I) is a compound having the following formula:

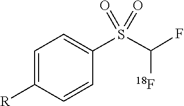

wherein R is selected from the group consisting of Bu, nitro and Cl, preferably wherein R is Cl.

Typically, the compound of formula (I) is a compound having the following formula:

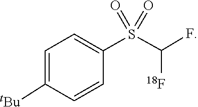 [SMILES: CC(C)(C)c1ccc(S(=O)(=O)C(F)[18F])cc1]

Preferably, the compound of formula (I) is a compound having the following formula:

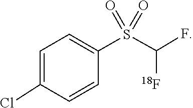 [SMILES: O=S(=O)(c1ccc(Cl)cc1)C(F)[18F]]

The compound of formula (I) is typically prepared from a compound of formula (II):

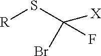

wherein R and X are as defined above. Typically the compound of formula (I) is prepared by contacting the compound of formula (II) with a source of ¹⁸F, and performing oxidation on the sulfur atom.

Any suitable source of ¹⁸F may be used. Typically the source of ¹⁸F is a source of ¹⁸F⁻. As will be understood by the skilled person the ¹⁸F⁻ will typically be present in the form of a salt, with a counter cation. Any suitable counter cation may be used. Typically, the counter cation is a quaternary ammonium cation, for instance tetrabutylammonium, or an alkali metal cation, for instance Cs⁺ or K⁺, or a proton, H⁺. Preferably, the source of ¹⁸F⁻ comprises an alkali metal or ammonium salt of ¹⁸F⁻.

When an alkali metal cation is employed, the alkali metal cation may be complexed in a cryptand, for instance aminopolyether 2.2.2 (K₂₂₂), which is commercially available as Kryptofix-222. Thus, the source of ¹⁸F⁻ may further comprise a cryptand ligand. Advantageously, the addition of such a cryptand enables the fluoride ion ¹⁸F⁻ to be solubilized in a polar aprotic solvent, for instance acetonitrile or DMF. It also enables the formation of a ‘naked fluoride ion’ as a KF—K₂₂₂ complex. In one embodiment, therefore, the source of ¹⁸F⁻ is a K[¹⁸F]F—K₂₂₂ complex. Alternatively, the source of ¹⁸F⁻ may be [¹⁸F]TEAF (tetraethylammonium fluoride), [¹⁸F]TBAF (tetrabutylammonium fluoride), [¹⁸F]CsF, or [¹⁸F]HF. Typically, ¹⁸F⁻ is present as K[¹⁸F]F—K₂₂₂ or [¹⁸F]HF. More typically, ¹⁸F⁻ is present as K[¹⁸F]F—K₂₂₂.

For instance, the compound of formula (I) may be prepared by contacting a compound of formula (II) with a source of ¹⁸F⁻, typically with a K[¹⁸F]F—K₂₂₂ complex.

The oxidation reaction may be any reaction suitable to oxidise the bridging sulfur atom to a sulfone. This is typically achieved using an oxidising agent. Suitable oxidising agents are known to the skilled person and include NaIO₄/RuCl₃, meta-chloroperoxybenzoic acid (mCPBA), oxone, KMnO₄/MnO₂ and H₂O₂. Typically, the oxidising agent is NaIO₄/RuCl₃.

Typically, the compound of formula (I) is prepared by a first step of contacting the compound of formula (II) with a source of ¹⁸F, and a second step of performing oxidation on the sulfur atom. The first and second steps may be performed without isolation or purification of any intermediate, i.e. may be performed as a two-step one-pot process. This process is typically quick to perform, providing the compound of formula (I) in a suitable timeframe to give the ultimate ¹⁸F labelled products with suitable radioactivity for subsequent use, e.g. as PET ligands.

Typically, the compound of formula (I) is prepared by a first step of contacting the compound of formula (II) with a source of ¹⁸F⁻, preferably with a K[¹⁸F]F—K₂₂₂ complex, and a second step of performing oxidation on the sulfur atom, typically using an oxidising agent selected from the group consisting of NaIO₄/RuCl₃, meta-chloroperoxybenzoic acid (mCPBA), oxone, KMnO₄/MnO₂ and H₂O₂, preferably where the oxidising agent is NaIO₄/RuCl₃.

### ¹⁸F-Labelled Products

The invention also provides a compound comprising an ¹⁸F-difluoromethyl group bonded to nitrogen (i.e. the moiety N—CHF¹⁸F). Typically, the N atom is comprised in an N-heterocyclic or N-heteroaromatic ring. For instance, the N atom may be comprised in a substituted or unsubstituted pyridyl, substituted or unsubstituted pyrazinyl, substituted or unsubstituted pyrimidinyl, substituted or unsubstituted pyridazinyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, or a substituted or unsubstituted carbazolyl group.

Preferably, the N atom is comprised in a substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl or a substituted or unsubstituted quinolinyl group. The substituents on the N-heterocyclic or N-heteroaromatic ring may be 1, 2 or 3 substituents selected from those listed above for the aromatic group.

The compound comprising an ¹⁸F-difluoromethyl group bonded to nitrogen may be selected from the following compounds:

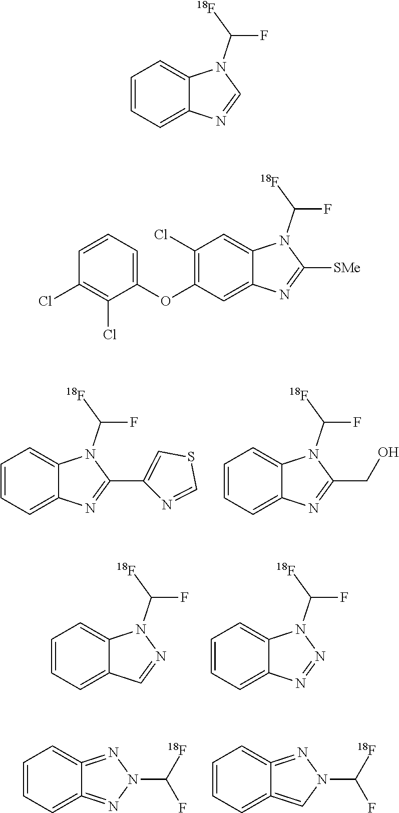

The invention also provides a compound comprising a gem-¹⁸F-difluorocyclopropane moiety. Thus, the compound may be a compound having the following formula:

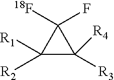

wherein R₁, R₂, R₃ and R₄ are each independently selected from the group consisting of hydrogent, substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Typically, R₁, R₂, R₃ and R₄ are each independently selected from the group consisting of hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl and substituted or unsubstituted heterocyclyl. For instance, R₁, R₂, R₃ and R₄ may each independently selected from the group consisting of unsubstituted C₁₋₆ alkyl, unsubstituted C₂₋₆ alkenyl, unsubstituted C₂₋₆ alkynyl, unsubstituted aryl and unsubstituted heteroaryl.

For instance, the compound may be

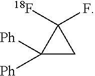 [SMILES: FC1([18F])CC1(c1ccccc1)c1ccccc1]

The invention also provides a compound comprising an ¹⁸F-difluoromethyl group bonded to an alkyne. Thus, the compound may be a compound having the following formula:

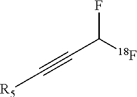

where R₅ is selected from the group consisting of hydrogent, substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Typically, R₅ is selected from the group consisting of hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl and substituted or unsubstituted heterocyclyl.

The invention also provides a compound comprising an ¹⁸F-difluoromethyl group bonded to an oxygen atom (i.e. the i.e. the moiety O—CHF¹⁸F). Typically, the O atom is bonded directly to an aromatic group or benzylic group. For instance, the O atom may be bonded directly to substituted or unsubstituted aryl group as described herein, or a substituted or unsubstituted heteroaryl group or a substituted or unsubstituted benzyl group as described herein.

The O atom may be bonded directly to substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl. Preferably, the O atom is bonded directly to a substituted or unsubstituted phenyl group. The substituents on the aryl group may be 1, 2 or 3 substituents selected from those listed above for the aromatic group.

The O atom may be bonded directly to a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted thiophenyl, substituted or unsubstituted benzofuranyl, substituted or unsubstituted dibenzofuranyl, substituted or unsubstituted benzothiphenyl, substituted or unsubstituted dibenzothiophenyl, substituted or unsubstituted xanthenyl and substituted or unsubstituted carbazolyl. The O atom may be bonded directly to a substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl or substituted or unsubstituted quinolinyl group. The substituents on the heteroaryl group may be 1, 2 or 3 substituents selected from those listed above for the aromatic group.

The comprising an ¹⁸F-difluoromethyl group bonded to an oxygen atom may be selected from the following compounds:

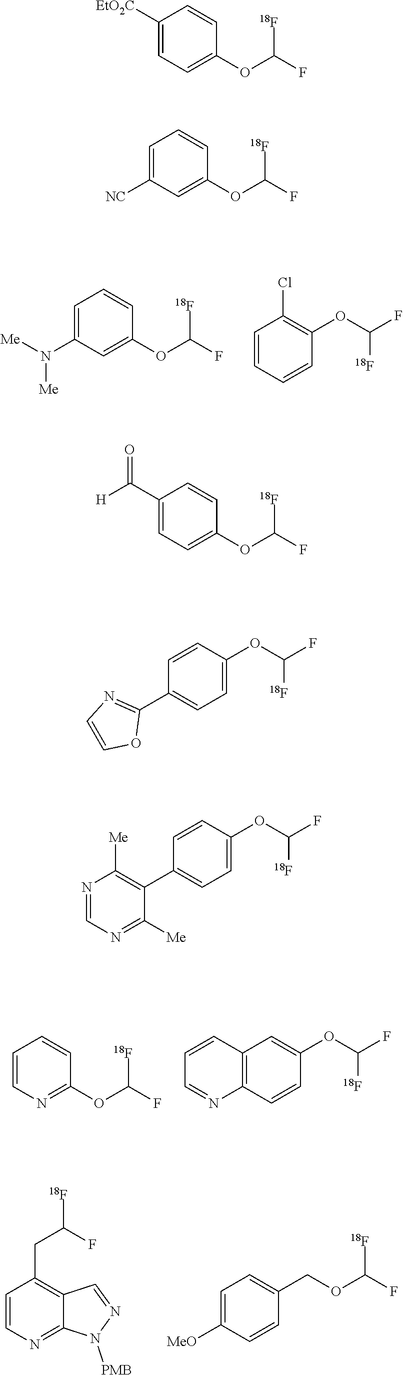

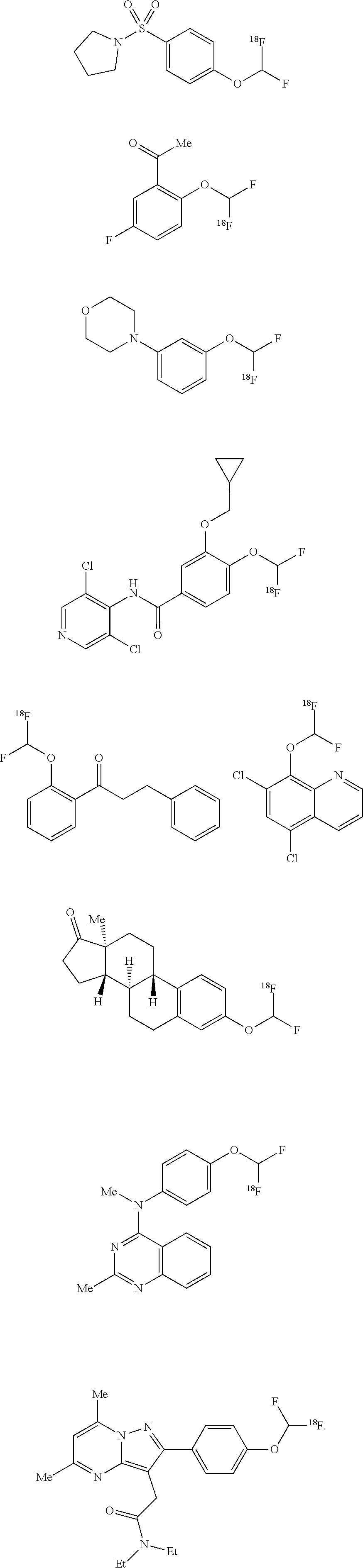

The invention also provides a compound comprising an ¹⁸F-difluoromethyl group bonded to a sulfur atom, (i.e. the i.e. the moiety S—CHF¹⁸F). For instance, the S atom may be bonded directly to substituted or unsubstituted aryl group as described herein, or a substituted or unsubstituted heteroaryl group as described herein, or a substituted or unsubstituted benzyl group as described herein.

The S atom may be bonded directly to substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl. Preferably, Ar represents a substituted or unsubstituted phenyl group. The substituents on the aryl group may be 1, 2 or 3 substituents selected from those listed above for the aromatic group.

The S atom may be bonded directly to a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted thiophenyl, substituted or unsubstituted benzofuranyl, substituted or unsubstituted dibenzofuranyl, substituted or unsubstituted benzothiphenyl, substituted or unsubstituted dibenzothiophenyl, substituted or unsubstituted xanthenyl and substituted or unsubstituted carbazolyl. The S atom may be bonded directly to a substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl or substituted or unsubstituted quinolinyl group. The substituents on the heteroaryl group may be 1, 2 or 3 substituents selected from those listed above for the aromatic group.

The compound may be selected from the following compounds:

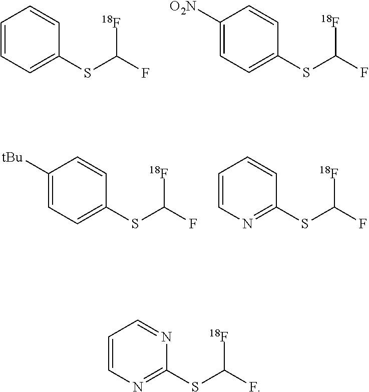

The compound may be a positron emission tomography (PET) ligand.

The invention also provides a compound obtainable by the process as described herein.

### Medical Uses

The invention also provides a pharmaceutical composition comprising (i) a compound comprising an ¹⁸F-difluoromethyl group or an ¹⁸F-difluoromethylene group as described herein, or a pharmaceutically acceptable salt thereof, and optionally (ii) one or more pharmaceutically acceptable ingredients.

Suitable pharmaceutically acceptable ingredients are well known to those skilled in the art and include pharmaceutically acceptable carriers (e.g. a saline solution, an isotonic solution), diluents, excipients, adjuvants, fillers, buffers, preservatives, anti-oxidants, lubricants, stabilisers, solubilisers, surfactants (e.g. wetting agents), masking agents, colouring agents, flavouring agents and sweetening agents. Suitable carriers, diluents, excipients, etc. can be found in standard pharmaceutical texts. See, for example, Handbook for Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

A pharmaceutical composition may be in the form of (i.e. be formulated as) a liquid, a solution or a suspension (e.g. an aqueous or a non-aqueous solution), an emulsion (e.g. oil-in-water, water-in-oil), an elixir, a syrup, an electuary, a tablet (e.g. coated tablets), granules, a powder, a lozenge, a pastille, a capsule (e.g. hard and soft gelatine capsules), a pill, an ampoule, a bolus, a tincture, a gel, a paste or an oil.

Typically the pharmaceutical composition is suitable for parenteral administration. A pharmaceutical composition suitable for parenteral administration (e.g. by injection) may include an aqueous or non-aqueous, sterile liquid in which the particles employed in the invention are dissolved or suspended. Such liquids may additionally contain other pharmaceutically acceptable ingredients, such as anti-oxidants, buffers, preservatives, stabilisers, bacteriostats, suspending agents, thickening agents, and solutes that render the formulation isotonic with the blood (or other relevant bodily fluid) of the intended recipient. Examples of excipients include water, alcohols, polyols, glycerol, vegetable oils, and the like. Examples of suitable isotonic solutions for use in such formulations include Sodium Chloride Injection, Ringer's Solution or Lactated Ringer's Injection.

The invention also provides a compound comprising an ¹⁸F-difluoromethyl group or an ¹⁸F-difluoromethylene group as described herein for use in a method for treatment of the human or animal body by therapy or for use in a diagnostic method practised on the human or animal body.

The invention also provides a method of treatment comprising administering a therapeutically effective amount of a compound comprising an ¹⁸F-difluoromethyl group or an ¹⁸F-difluoromethylene group as described to a subject.

The invention also provides the use of a compound comprising an ¹⁸F-difluoromethyl group or an ¹⁸F-difluoromethylene group as described herein in the manufacture of a medicament.

The invention also provides a method of imaging a subject, comprising administering to the subject a compound comprising an ¹⁸F-difluoromethyl group or an ¹⁸F-difluoromethylene group as described herein or a pharmaceutically acceptable salt thereof, and imaging the subject by positron emission tomography (PET). The method may comprise administering to the subject a pharmaceutical composition as described herein.

The invention is further described in the following Examples.

### EXAMPLES

In 2019, Genicot, Luxen and co-workers disclosed the radiosynthesis of a [¹⁸F]difluoromethyl sulfone reagent with application to the C—H ¹⁸F-difluoromethylation of heteroarenes (see Zheng et al. Chlorodifluoromethyl phenyl sulfone: a novel non-ozone-depleting substance-based difluorocarbene reagent for O- and N-difluoromethylations. Chem. Commun. 2007, 5149-5151). Inspired by this report, we felt [¹⁸F]chlorodifluoromethyl phenyl sulfone ([¹⁸F]1), a known [¹⁹F]DFC surrogate, was within reach. We proposed that ¹⁸F-fluorination of bromofluoromethylthioether followed by oxidation and chlorination could provide access to [¹⁸F]1 in three steps. Applying this three step radiosynthesis, we obtained [¹⁸F]1 in a radiochemical yield (RCY) of ˜1% and >99% (radio)chemical purity. With this reagent in hand, we illustrated for the first time the successful radiosynthesis of a [¹⁸F]DFC reagent (see Hine, J.; Porter, J. J. The Formation of Difluoromethylene from Difluoromethyl Phenyl Sulfone and Sodium Methoxide 1. J. Am. Chem. Soc. 1960, 82, 6178-6181). However, the complex radiosynthesis and the instability of [¹⁸F]1 were deemed as detrimental which would prevent the broader application of this chemistry. In search for an alternative [¹⁸F]DFC reagent which was chemically more stable, we became inspired by a 1960 report from Hine and Porter who described the release of DFC from PhSO₂CF₂H in basic conditions (see Fier et al. Synthesis of difluoromethyl ethers with difluoromethyltriflate. Angew. Chem. Int. Ed. 2013, 52, 2092-2095). We therefore investigated whether [¹⁸F]4-tBuPhSO₂CF₂H ([¹⁸F]2) could be exploited as a [¹⁸F]DFC reagent. With further optimization, [¹⁸F]2 was prepared in a non-decay corrected activity yield (AY) of 10%±2% (n=5) and MA of 10 GBq/μmol after isolation and reformulation (no decomposition observed, Scheme 1A). To evaluate whether [¹⁸F]2 could indeed be used as a [¹⁸F]DFC reagent, we examined the [¹⁸F]difluorocyclopropanation of 1,1-diphenylethylene (3a). Mechanistically, such a reaction would most likely proceed through a difluorocarbene pathway. Indeed, when [¹⁸F]2 was reacted with sodium hydroxide in presence of 3a we obtained difluorocyclopropane [¹⁸F]4a in 36% RCY (Scheme 1B).

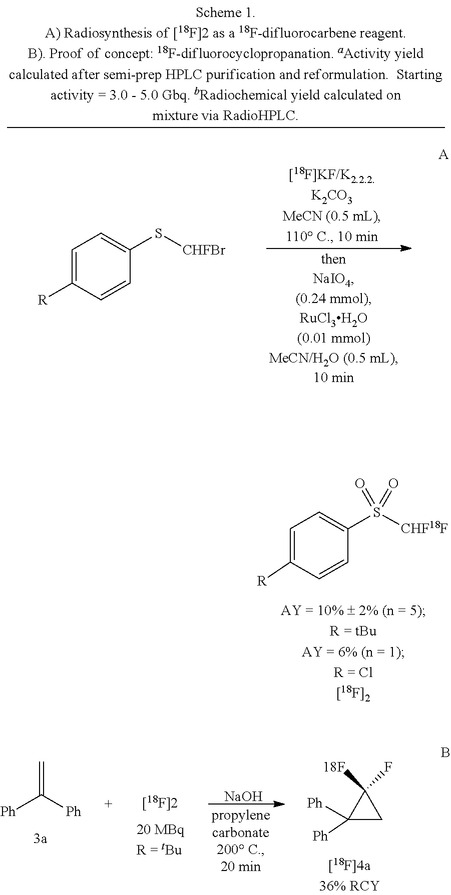

With the proof of concept in hand that illustrates [¹⁸F]2 is indeed a [¹⁸F]DFC reagent, we next focused on identifying milder reaction conditions for the difluoromethylation of (thio)phenols. We found that when 4-biphenyl phenol (3b) was treated with [¹⁸F]2 in a mixture of aqueous KOH and acetonitrile at 80° C. the difluoromethyl ether [¹⁸F]4b was obtained in 58% RCY (Table 1, entry 1). Doubled substrate concentration led to an increased RCY of 71% (Table 1, entry 2). When the amount of potassium hydroxide was changed the product [¹⁸F]4b was obtained in lower yield (Table 1, entries 3-4). Changing the solvent to DMSO had detrimental effects on the reaction outcome (Table 1, entry 5). Furthermore, when the reaction was performed in absence of water the desired product was not observed (Table 1, entry 6). Other attempts aimed at lowering the substrate loading led to lower radiochemical yields of [¹⁸F]4b (Table 1, entry 7).

TABLE 1
Optimization of the reaction conditions for the [¹⁸F]difluoromethylation
of phenol 3b.ª

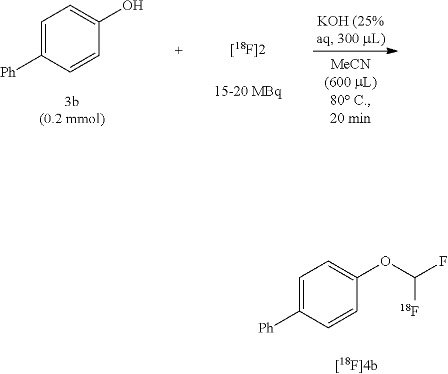 [SMILES: Oc1ccc(-c2ccccc2)cc1 ; FC([18F])Oc1ccc(-c2ccccc2)cc1]

| Entry | Alterations to conditions | RCY |
|---|---|---|
| 1 | 3b (0.1 mmol) | 58% |
| 2 | No alteration | 71% ± 10 |
| | | (n = 3) |
| 3 | KOH (25% aq, 50 μL) | Trace |
| 4 | KOH (25% aq, 150 μL) | 59% |
| 5 | DMSO (600 μL) instead of MeCN | 25% |
| 6 | KOH (3 mg) instead of KOH (aq) | 0% |
| 7 | 3b (0.1 mmol), KOH (25% aq, 150 μL), | 52% |
| | MeCN (300 μL) | |

^{a}Reaction conditions:
3b (0.2 mmol), KOH (25% aq, 300 μL), MeCN (600 μL), [¹⁸F]2 (15-20 MBq), 80° C., 20 min.
Radiochemical Yield calculated on crude mixture via RadioTLC and RadioHPLC.
n = number of reactions

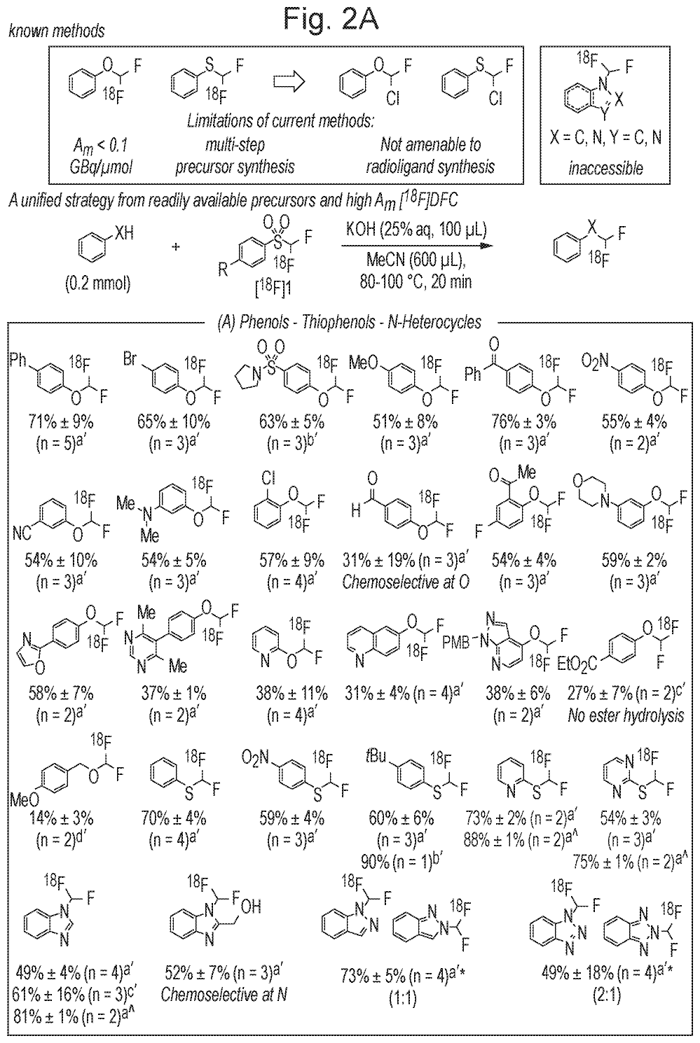

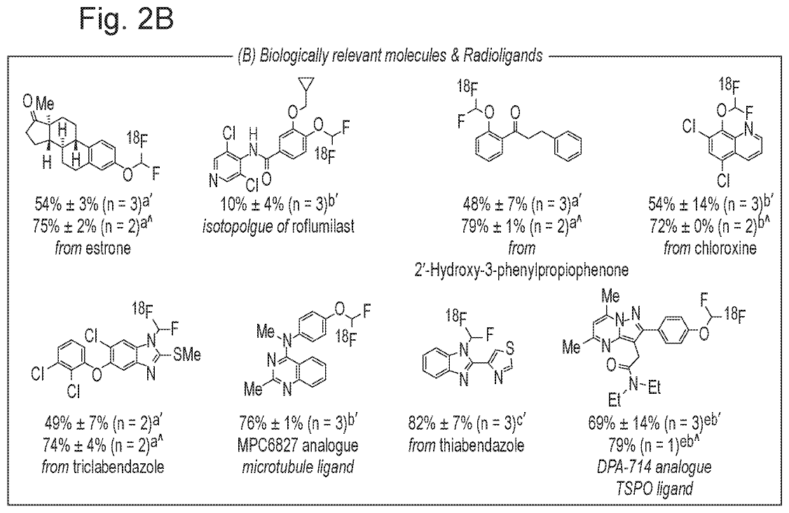

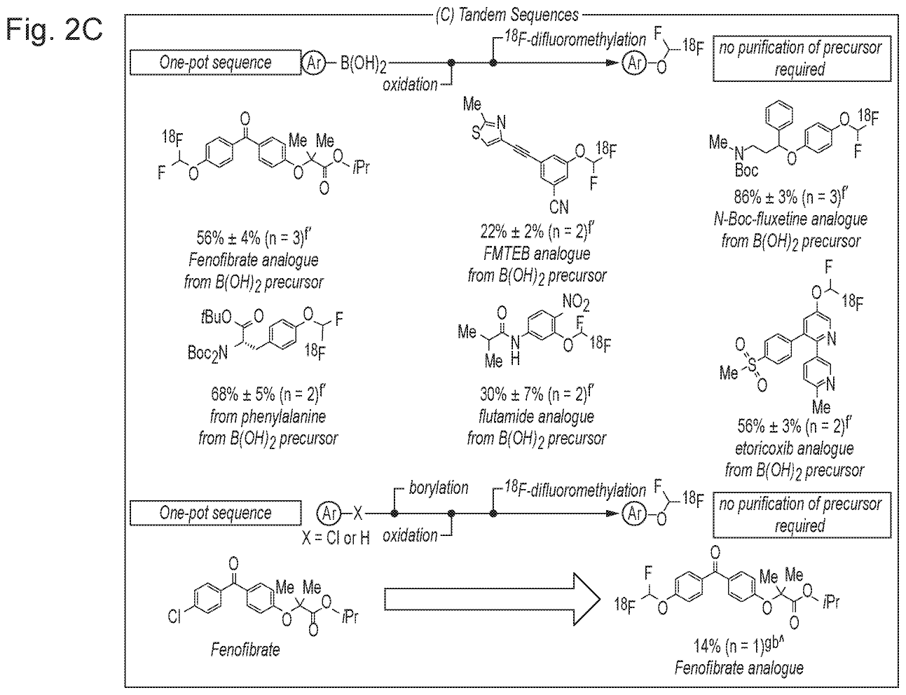

With the optimized reaction conditions in hand (Table 1, entry 2), we next evaluated the reaction on a broad scope of (thio)phenols (FIG. 2). Phenols with different electronic properties and different substitution patterns were all difluoromethylated in moderate to high RCY ([¹⁸F]4b-[¹⁸F]4k). Halide, ketone, nitro, nitrile, amine and aldehyde functionalities were all tolerated under the reaction conditions and remained untouched. Due to the prevalence of heteroarenes in drug/radiotracer scaffolds, we further investigated a series of both heteroaryl phenols as well as arenes substituted with heteroaryl functionalities. Phenols substituted with other heteroarenes such as oxazole and pyrimidine gave access to difluoromethyl ethers [¹⁸F]41 and [¹⁸F]4m in moderate to good RCY. 2-hydroxypyridine (3n), quinolin-6-ol (3o) and a fused pyrazolo-pyridine substrate (3p) underwent the desired transformation in moderate RCY. A representative aliphatic alcohol reacted in very low radiochemical yield under the standard reaction conditions ([¹⁸F]4q).

Inspired by a recent report from Hartwig and co-workers we investigated a one-pot procedure from readily available boronic acids (see Teare et al. Radiosynthesis and evaluation of [¹⁸F]Selectfluor bis (triflate). Angew. Chem. Int. Ed. 2010, 49, 6821-6824; Teare et al. Synthesis and reactivity of [¹⁸F]—N-fluorobenzenesulfonimide. Chem. Commun. 2007, 2330-2332; Verhoog et al. ¹⁸F-trifluoromethylation of unmodified peptides with 5-¹⁸F-(trifluoromethyl) dibenzothiophenium trifluoromethanesulfonate. J. Am. Chem. Soc. 2018, 140, 1572-1575). Indeed, when boronic acid 5b was first treated with urea hydroperoxide and then without further purification reacted under the standard reaction conditions, the desired [¹⁸F]difluoromethylether [¹⁸F]4b was obtained in 40% RCY. This one-pot procedure from aryl boron precursors was seen as advantageous, given the propensity of methods to install such a motif in a late-stage fashion and its applicability to F-18 radiochemistry.

Next, we turned our attention to the ¹⁸F-difluoromethylation of thiophenols. Independent of their electronic properties or substituents a range of [¹⁸F]difluoromethylthioethers were prepared in high RCY ([¹⁸F]4r-[¹⁸F]4t). A representative heteroaryl thiophenol was more reactive than the corresponding phenol and gave access to [¹⁸F]4u in high RCY. N-heterocycles, such as benzimidazole (3v) required slightly modified reaction conditions to undergo ¹⁸F-difluoromethylation in good RCY. When the solvent was changed to DMF and sodium hydride was used as base, [¹⁸F]4v was obtained in 69% RCY.

Given the absence in knowledge of DFC reactivity in the context of F-18 radiochemistry, we felt compelled to study its reactivity towards competing sites of insertion or whether these additional sites could terminate reactivity altogether. We felt that evaluation of the relative reactivity profiles of [¹⁸F]DFC derived from [¹⁸F]2 through simple competition experiments would provide insightful information, formative for end-users of this chemistry who will likely employ it for complex scaffolds. Competition experiments were conducted between 3b and 3t in order to determine which bond (N—H or O—H) would more readily react. Under the standard reaction conditions both substrates underwent ¹⁸F-difluoromethylation and no selectivity was observed. However, a competition experiment between 3e and 3q revealed that under the standard reaction conditions phenols react selectively and therefore might not require protection of aliphatic alcohols, among radiotracers which contain both motifs.

The synthesis and application of ¹⁸F-reagents pioneered by our group and others has significantly changed the landscape of F-18 radiochemistry. The disclosure of F-18 isotopologues of commonly used F-19 reagents, such as CuCF₃, Selectfluor, NFSI, and most recently Umemoto and Langlois reagents have helped bridge the gap between F-19 and F-18 developments (Kee et al. ¹⁸F-Trifluoromethanesulfinate enables Direct C—H ¹⁸F-Trifluoromethylation of Native Aromatic Residues in Peptides. J. Am. Chem. Soc. 2020, 142, 1180-1185; Zheng et al. Difluorocarbene-Derived Trifluoromethylthiolation and [¹⁸F]Trifluoromethylthiolation of Aliphatic Electrophiles. Angew. Chem. Int. Ed. 2015, 54, 13236-13240). Unlocking these F-18 reagents has allowed the field of F-18 radiochemistry to tremendously benefit from advancements in the field of F-19 organofluorine chemistry which has accelerated at a much faster pace. Despite these advancements, the use of F-18 reagents often impose additional limitations such as long radiosyntheses involving multiple purification steps, which in many instances result in low MA products. With the radiosynthesis of [¹⁸F]2 we were keen to address these challenges. After careful optimization we were successful in obtaining [¹⁸F]2 in high MA (131±29 GBq/μmol) for [¹⁸F]2 with R=Cl (see scheme below).

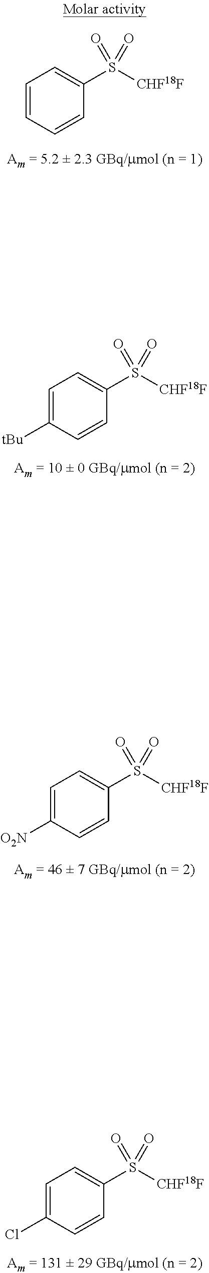

Given our success in developing a two-step-one-pot ¹⁸F-difluoromethylation protocol from 5b, we postulated on whether a telescoped radiosynthesis of [¹⁸F]4b from [¹⁸F]fluoride was possible. This would significantly reduce the overall synthesis time in addition to alleviating the requirement for additional cumbersome purification steps. Remarkably, we were successful in this endeavor and obtained [¹⁸F]2 after 15 minutes total reaction time in an RCY of 36% from bromofluoromethylthioether. Notably, when [¹⁸F]2 was used without HPLC purification and exposed to the standard reaction conditions, the desired difluoromethylether [¹⁸F]4b was obtained in 31% RCY.

In conclusion, we have disseminated the first protocol enabling access to [¹⁸F]DFC, a highly common reactive intermediate in synthetic chemistry. In this report we illustrate the synthesis of a [¹⁸F]DFC reagent, [¹⁸F]2 which allows controlled [¹⁸F]DFC insertion into a variety of X—H (X═O, S, N) bonds. This method is a new and valuable tool to accelerate the discovery of novel PET ligands with more stable fluorinated motifs. The strategy required the novel ¹⁸F reagent [¹⁸F]2, which was prepared in a two-step-one pot protocol (fluorination followed by oxidation) from [¹⁸F]fluoride. We illustrated that a telescoped synthesis of [¹⁸F]4b from was feasible and that [¹⁸F]4b was also accessible from an aryl boronic acid in a one pot fashion. In view of the number of reactions relying on difluorocarbene-type reagents, access to the first [¹⁸F]DFC reagent, [¹⁸F]2 will likely have a considerable impact on the radiochemical space which is being explored for PET applications. Specifically, it also provides an alternative entry point to access more stable analogues of ¹⁸F radiotracers which rely on ¹⁸F-alkylation strategies of which the resulting alkylation products readily undergo radiodefluorination.

### Supporting Information for Example 1

### Mechanistic Experiments

### Deuteration of 1-(tert-butyl)-4-((difluoromethyl)sulfonyl)benzene

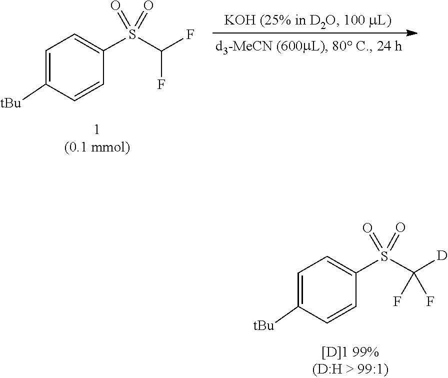 [SMILES: CC(C)(C)c1ccc(S(=O)(=O)C(F)F)cc1 ; [2H]C(F)(F)S(=O)(=O)c1ccc(C(C)(C)C)cc1]

1-(Tert-butyl)-4-((difluoromethyl)sulfonyl)benzene (24.8 mg, 0.1 mmol, 1.0 equiv) was dissolved in d₃-MeCN (600 μL) in a 7 mL vial. To this solution was added KOH (25% w/w in D₂O, 100 μL) at room temperature. The mixture was heated to 80° C. for 24 hours and cooled to room temperature. The mixture was diluted with d₆-DMSO (2.0 mL) and 4-fluoroanisole (11.3 μL, 0.1 mmol, 1.0 equiv) was added as an internal standard. The mixture was analysed (conversion and D:H ratio) by quantitative ¹⁹F NMR spectroscopy.

### Difluoromethylation of Sodium Benzenesulfinate

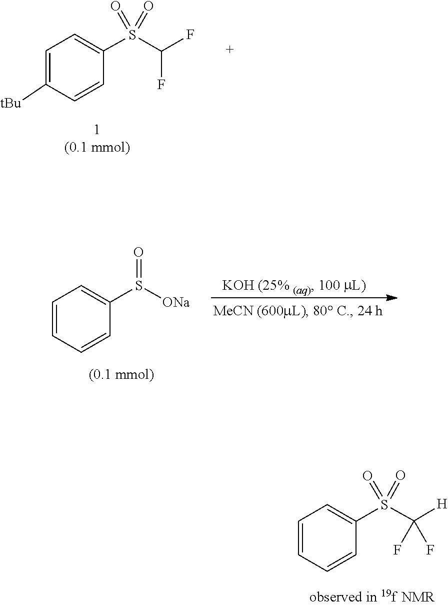

1-(tert-butyl)-4-((difluoromethyl)sulfonyl)benzene (24.8 mg, 0.1 mmol, 1.0 equiv) and sodium benzenesulfinate (16.4 mg, 0.1 mmol, 1 equiv) were added to a 7 mL vial in MeCN (600 μL). To this solution was added KOH (25% w/w in H₂O, 100 μL) at room temperature. The mixture was heated to 80° C. for 24 hours and cooled to room temperature. The mixture was diluted with d₆-DMSO (2.0 mL) and 4-fluoroanisole (11.3 μL, 0.1 mmol, 1.0 equiv) was added as an internal standard. The mixture was analysed by quantitative ¹⁹F NMR spectroscopy.

### Difluoromethylation of Phenol in Deuterated Solvents

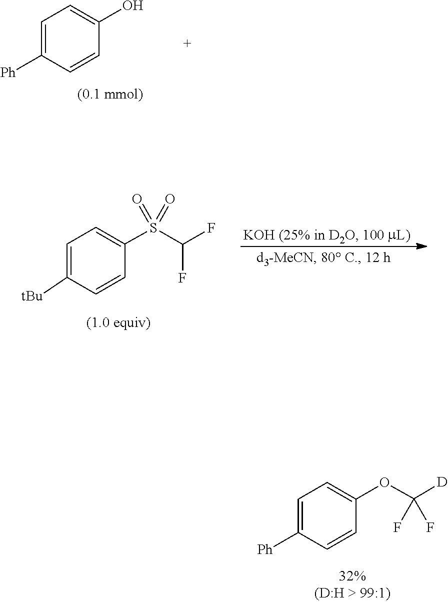 [SMILES: Oc1ccc(-c2ccccc2)cc1 ; CC(C)(C)c1ccc(S(=O)(=O)C(F)F)cc1 ; [2H]C(F)(F)Oc1ccc(-c2ccccc2)cc1]

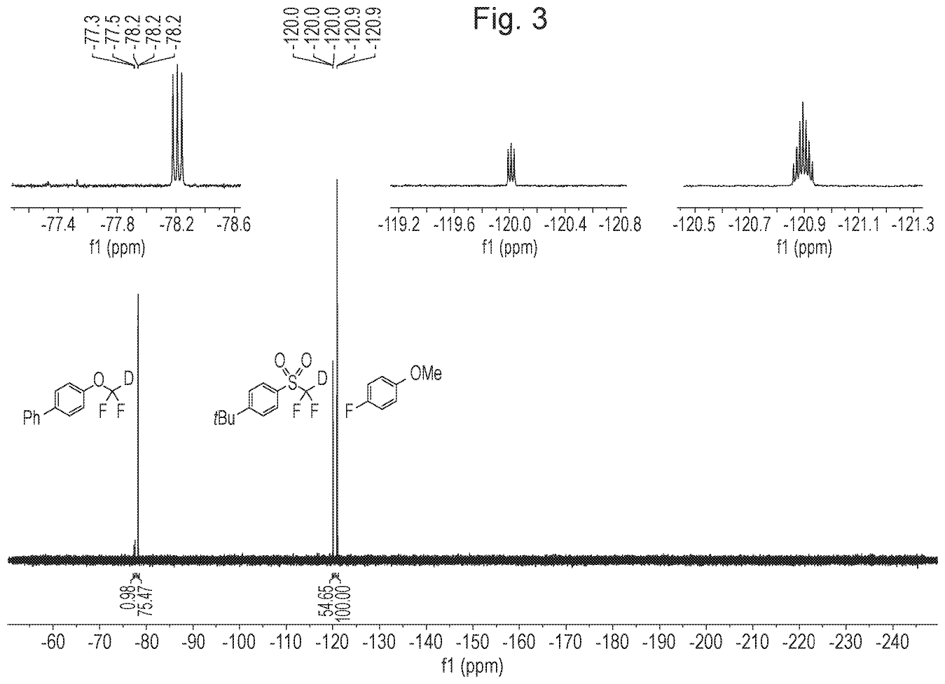

1-(tert-butyl)-4-((difluoromethyl)sulfonyl)benzene (24.8 mg, 0.1 mmol, 1.0 equiv) and 4-phenylphenol (17.0 mg, 0.1 mmol, 1 equiv) were dissolved in MeCN (600 μL) in a 7 mL vial. To this solution was added KOH (25% w/w in D₂O, 100 μL) at room temperature. The mixture was stirred at 80° C. for 12 hours and cooled to room temperature. The mixture was diluted with d₆-DMSO (2.0 mL) and 4-fluoroanisole (11.3 μL, 0.1 mmol, 1.0 equiv) was added as an internal standard. The mixture was analysed (conversion and D:H ratio) by quantitative ¹⁹F NMR spectroscopy (see FIG. 3).

### Difluoromethylation of Phenol with [D]1 in Non-Deuterated Solvents

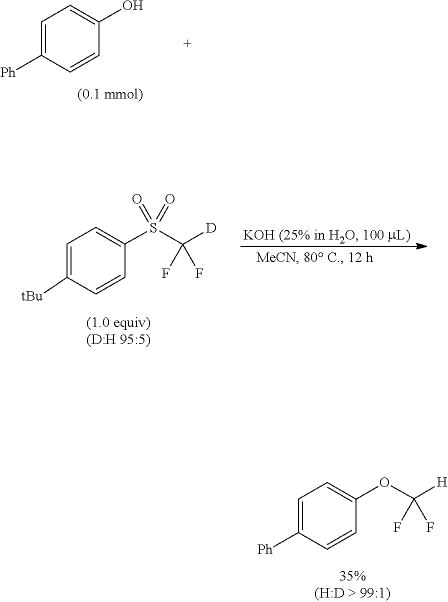 [SMILES: Oc1ccc(-c2ccccc2)cc1 ; [2H]C(F)(F)S(=O)(=O)c1ccc(C(C)(C)C)cc1 ; FC(F)Oc1ccc(-c2ccccc2)cc1]

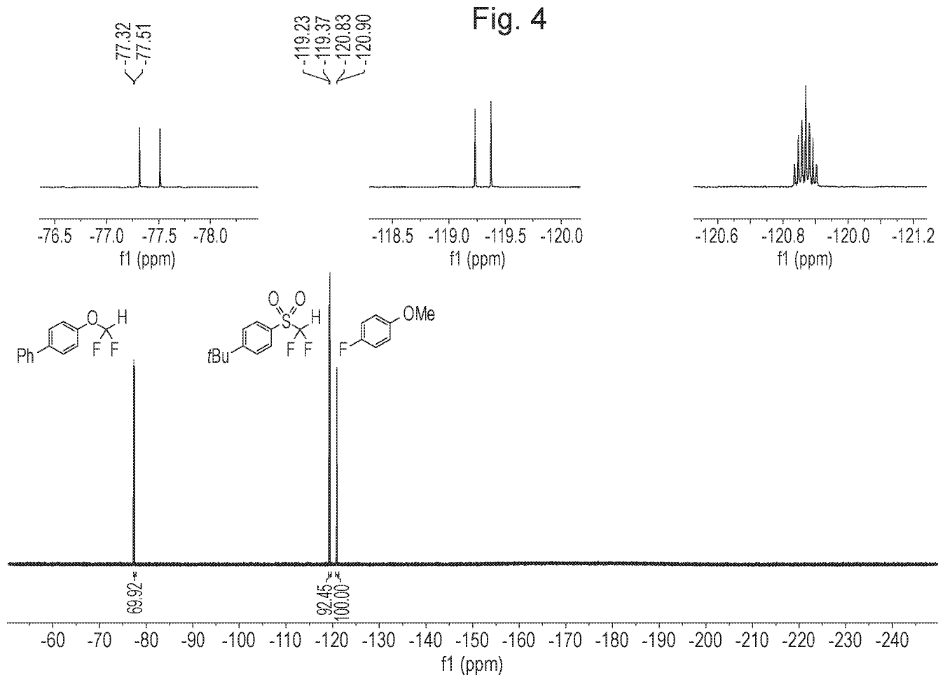

1-(Tert-butyl)-4-((difluoromethyl-d)sulfonyl)benzene (24.9 mg, 0.1 mmol, 1.0 equiv) and 4-phenylphenol (17.0 mg, 0.1 mmol, 1.0 equiv) were added to a 7 mL vial in MeCN (600 μL). To this solution was added KOH (25% w/w in H₂O, 100 μL) at room temperature. The mixture was heated to 80° C. for 12 hours and cooled to room temperature. The mixture was diluted with d₆-DMSO (2.0 mL) and 4-fluoroanisole (11.3 μL, 0.1 mmol, 1.0 equiv) was added as an internal standard. The mixture was analysed (conversion and D:H ratio) by quantitative ¹⁹F NMR spectroscopy (see FIG. 4).

### Difluorocyclopropanation of 1,1-diphenylethylene

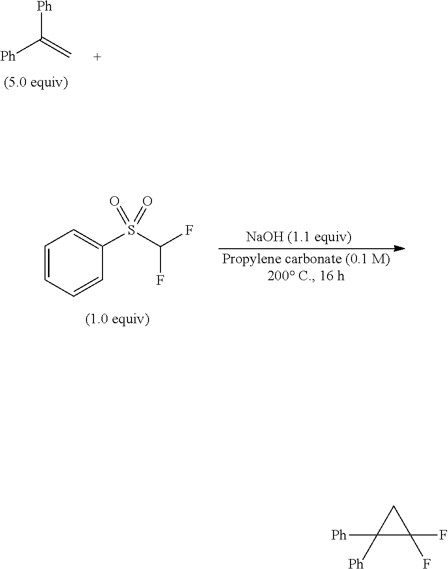

((Difluoromethyl)sulfonyl)benzene (19 mg, 0.1 mmol, 1.0 equiv) was added to a suspension of 1,1-diphenylethylene (90 mg, 0.5 mmol, 5.0 equiv) and sodium hydroxide (4.5 mg, 0.11 mmol, 1.1 equiv) in dry propylene carbonate (5.0 mL) at room temperature. The mixture was heated to 200° C. for 16 hours and cooled to room temperature. The mixture was diluted with ethanol (2.0 mL) and α,α,α-trifluorotolouene (123 μL, 1.0 mmol, 10.0 equiv) was added as an internal standard. The mixture was analysed by quantitative ¹⁹F NMR spectroscopy.

### Difluoromethylation of Sodium Phenoxide

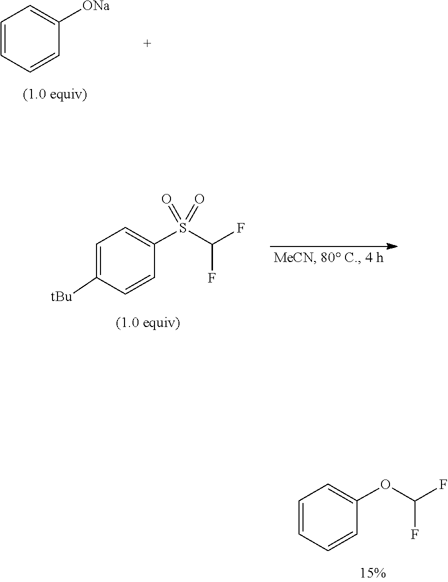

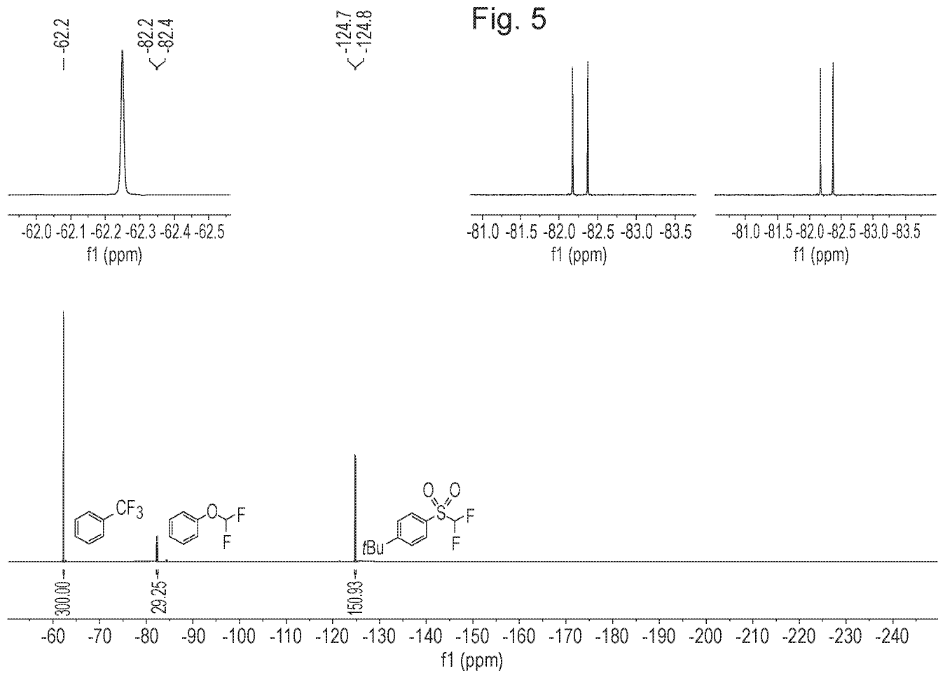

1-(Tert-butyl)-4-((difluoromethyl)sulfonyl)benzene (248 mg, 1.0 mmol, 1.0 equiv) was added to a suspension of sodium phenoxide (116 mg, 1.0 mmol, 1.0 equiv) in dry MeCN (10 mL) at room temperature. The mixture was heated to 80° C. for 4 hours and cooled to room temperature. The mixture was diluted with ethanol (10.0 mL) and α,α,α-trifluorotolouene (123 μL, 1.0 mmol, 1.0 equiv) was added as an internal standard. The mixture was analysed by quantitative ¹⁹F NMR spectroscopy (see FIG. 5).

### Difluoromethylation of Sodium Thiophenolate

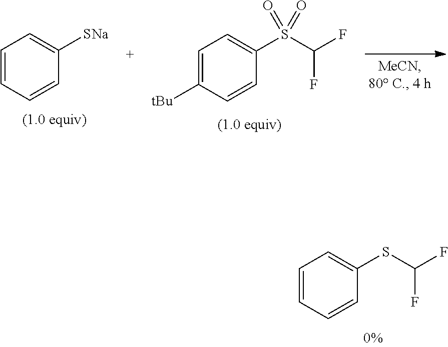

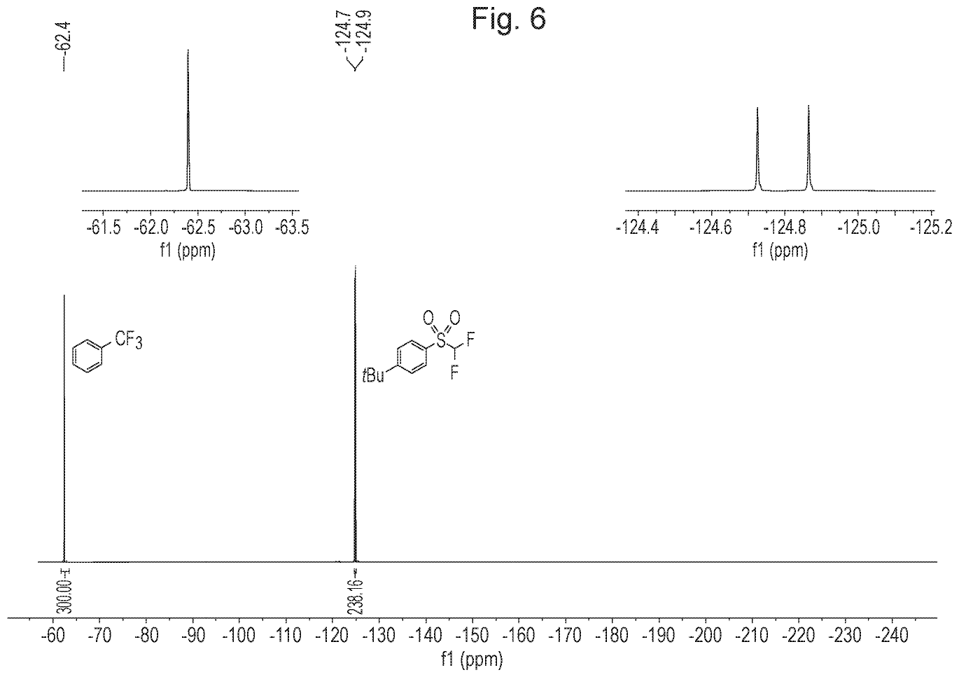

1-(Tert-butyl)-4-((difluoromethyl)sulfonyl)benzene (248 mg, 1.0 mmol, 1.0 equiv) was added to a suspension of sodium thiophenolate (132 mg, 1.0 mmol, 1.0 equiv) in dry MeCN (10 mL) at room temperature. The mixture was heated to 80° C. for 4 hours and cooled to room temperature. The mixture was diluted with ethanol (10.0 mL) and α,α,α-trifluorotolouene (123 μL, 1.0 mmol, 1.0 equiv) was added as an internal standard. The mixture was analysed by quantitative ¹⁹F NMR spectroscopy (see FIG. 6).

### Difluoromethylation of Phenol Under Substoichiometric Conditions

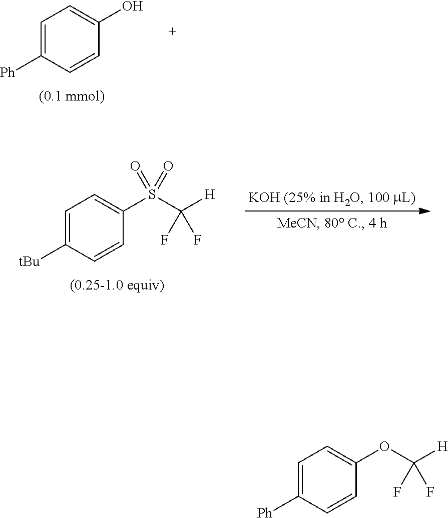 [SMILES: Oc1ccc(-c2ccccc2)cc1 ; CC(C)(C)c1ccc(S(=O)(=O)C(F)F)cc1 ; FC(F)Oc1ccc(-c2ccccc2)cc1]

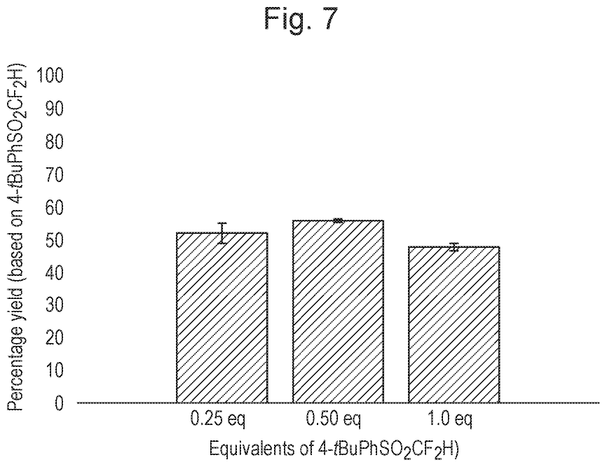

1-(tert-butyl)-4-((difluoromethyl)sulfonyl)benzene (24.8 mg, 0.1 mmol, 1.0 equiv) and 4-phenylphenol (17.0 mg, 0.1 mmol, 1.0 equiv) were added to a 7 mL vial in MeCN (600 μL). To this solution was added KOH (25% w/w in H₂O, 100 μL) at room temperature. The mixture was heated to 80° C. for 24 hours and cooled to room temperature. The mixture was diluted with d₆-DMSO (2.0 mL) and 4-fluoroanisole (11.3 μL, 0.1 mmol, 1.0 equiv) was added as an internal standard. The mixture was analysed by quantitative ¹⁹F NMR spectroscopy. Yields shown are calculated based on quantitative ¹⁹F NMR spectroscopy and the average of three runs (see FIG. 7).

### Synthesis of (bromofluoromethyl)(4-(tert-butyl)phenyl)sulfane

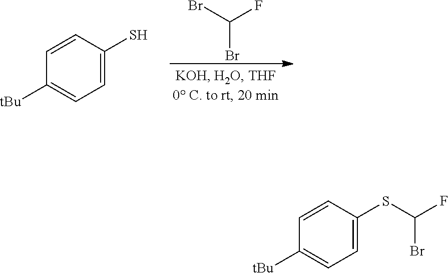

(Bromofluoromethyl)(4-(tert-butyl)phenyl)sulfane was prepared according to a modified literature procedure:^{[3]} A solution of KOH (3.36 g, 60 mmol, 10.0 equiv) in H₂O (8.0 mL) was added to a round-bottomed flask (under air) and cooled to 0° C. To this solution was added, 4-(tert-butyl)benzenethiol (6.0 mmol, 1.0 equiv) in THF (6.0 mL). The resulting mixture was warmed to room temperature and stirred for 20 minutes. A solution of dibromofluoromethane (1.43 mL, 18.0 mmol, 3.0 equiv) in THF (2.0 ml) was subsequently added dropwise. The reaction mixture was stirred at room temperature for a 20 minutes, after which it was quenched by the addition of H₂O (40 mL). The crude product was extracted with DCM (3×50 mL). The combined organic layers were washed with brine (50 mL) and dried over MgSO₄. After filtration, the crude compound was concentrated in vacuo. The crude product was purified by silica gel column chromatography (pentane) and (bromofluoromethyl)(4-(tert-butyl)phenyl)sulfane was isolated as a colourless oil. ¹H NMR (400 MHz, CDCl₃) δ 7.55-7.51 (m, 2H), 7.46-7.42 (m, 2H), 7.21 (d, J=56.10 Hz) 1.35 (s, 9H); ¹³C NMR (101 MHz, CDCl₃) δ 153.5, 134.5, 127.3, 126.5, 92.9 (d, J=293.9 Hz), 34.9, 31.2; ¹⁹F NMR (376 MHz, CDCl₃) δ −99.5 (d, J=56.2 Hz); HRMS: the compound did not ionise under various ionisation techniques; IR (neat): 2963, 2869, 1596, 1489, 1461, 1398, 1364, 1294, 1268, 1175, 1151, 1117, 995, 830.

Note: prolonged reaction times lead to consumption of the product and result in (fluoromethylene)bis((4-(tert-butyl)phenyl)sulfane).

### Synthesis of Reference Compounds

A number of compounds are known in literature and were synthesised accordingly. Roflumilast was obtained from a commercial supplier (ABCR).

### General Procedure 1 (GP1): Difluoromethylation of Phenols with Diethyl Bromodifluoromethylphosphonate

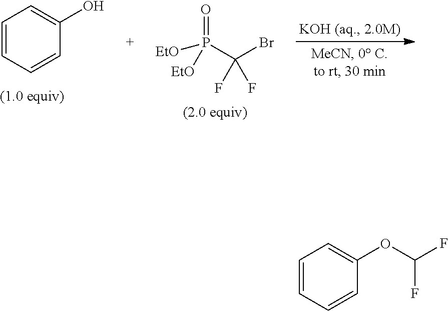 [SMILES: Oc1ccccc1 ; CCOP(=O)(OCC)C(F)(F)Br ; FC(F)Oc1ccccc1]

According to a modified literature procedure.^{[5]} Diethyl bromodifluoromethylphosphonate (356 μL, 2.0 mmol, 2.0 equiv) was added to a stirred mixture of phenol (1.0 mmol, 1.0 equiv) in MeCN (5.0 mL) and KOH (aq., 2.0 M, 5.0 mL) at 0° C. The mixture was allowed to warm to room temperature and stirred for 30 min or until complete consumption of starting material. The mixture was extracted with Et₂O (3×5.0 mL) and the organic layer was dried (Na₂SO₄), filtered and concentrated in vacuo. The crude product was purified by silica gel column chromatography.

### General Procedure 2 (GP2): Difluoromethylation of N-Heterocycles with Diethyl Bromodifluoromethylphosphonate

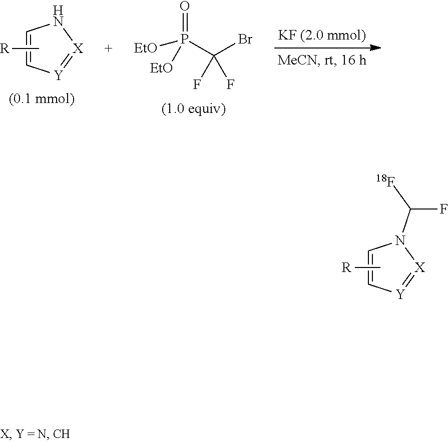

According to a modified literature procedure.^{[11]} Diethyl bromodifluoromethylphosphonate (178 μL, 1.0 mmol, 1.0 equiv) was added to a stirred mixture of N-heterocycle (1.0 mmol, 1.0 equiv) and KF (116 mg, 2.0 mmol, 2.0 equiv) in MeCN (8.0 mL). The mixture was stirred overnight and concentrated. The crude product was purified by silica gel column chromatography.

### 3-(difluoromethoxy)-N,N-dimethylaniline

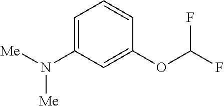 [SMILES: CN(C)c1cccc(OC(F)F)c1]

GP1 was followed (1.0 mmol scale) to yield compound (140 mg, 0.75 mmol, 75%) as a colourless oil. ¹H NMR (400 MHz, CDCl₃) δ 7.11-7.03 (m, 1H), 6.46-6.42 (m, 1H), 6.36-6.30 (m, 2H), 2.84 (s, 6H); ¹³C NMR (101 MHz, CDCl₃) δ 152.8 (t, J=2.7 Hz), 152.0, 130.1, 116.4 (t, J=257.3 Hz), 109.4, 106.2, 103.3, 40.3; ¹⁹F NMR (377 MHz, CDCl₃) δ −79.8 (d, J=74.9 Hz); HRMS (ESI-TOF): calculated for C₉H₁₁F₂NO ([M+H]⁺): 188.0881; found: 188.0881; IR (neat): 1683, 1507, 1441, 1357, 1214, 1176, 1158, 1119, 1041.

### 1-(2-(difluoromethoxy)-4-fluorophenyl)ethan-1-one

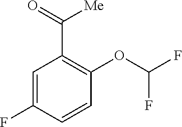 [SMILES: CC(=O)c1cc(F)ccc1OC(F)F]

GP1 was followed (1.0 mmol scale) to yield compound (135 mg, 0.66 mmol, 66%) as a white solid. ¹H NMR (400 MHz, CDCl₃) δ 7.81 (dd, J=8.79, 6.57 Hz, 1H), 6.97 (ddd, J=8.79, 7.54, 2.40 Hz, 1H), 6.93-6.87 (m, 1H), 6.62 (t, J=72.85 Hz, 1H), 2.58 (s, 3H); ¹³C NMR (101 MHz, CDCl₃) δ 196.7, 165.1 (d, J=255.5 Hz), 151.0 (dt, J=10.9, 3.2 Hz), 132.8 (d, J=10.3 Hz), 127.5 (d, J=3.6 Hz), 115.9 (t, J=261.5 Hz), 112.9 (d, J=21.2 Hz), 107.5 (d, J=25.6 Hz), 31.2; ¹⁹F NMR (377 MHz, CDCl₃) δ −81.7 (d, J=73.1 Hz), −103.1-−103.3 (m); HRMS: the compound did not ionise under various ionisation techniques; IR (neat): 3056, 1669, 1594, 1494, 1428, 1386, 1359, 1307, 1256, 1171, 1091, 1057, 1040, 985, MP: 52-54° C.

### 1-((4-(difluoromethoxy)phenyl)sulfonyl)pyrrolidine

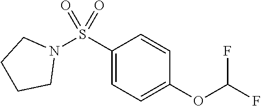 [SMILES: O=S(=O)(c1ccc(OC(F)F)cc1)N1CCCC1]

GP1 was followed (1.0 mmol scale) to yield compound (199 mg, 0.72 mmol, 72%) as a white solid. ¹H NMR (400 MHz, CDCl₃) δ 7.84-7.79 (m, 2H), 7.25-7.20 (m, 2H), 6.61 (t, J=72.79 Hz, 1H), 3.28-3.14 (m, 4H), 1.83-1.70 (m, 4H); ¹³C NMR (101 MHz, CDCl₃) δ 154.2 (t, J=2.7 Hz), 133.9, 129.7, 119.4, 115.3 (t, J=262.0 Hz), 48.0, 25.3; ¹⁹F NMR (377 MHz, CDCl₃) δ −82.1 (d, J=73.0 Hz); HRMS (ESI-TOF): calculated for C₁₁H₁₄F₂NO₃S ([M+H]⁺): 278.0657; found: 278.0658; IR (neat): 1598, 1493, 1398, 1351, 1329, 1299, 1245, 1206, 1168, 1094, 1070, 1047, 1009, 834, MP: 63-64° C.

### 4-(3-(difluoromethoxy)phenyl)morpholine

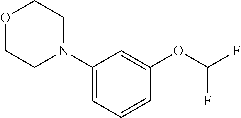 [SMILES: FC(F)Oc1cccc(N2CCOCC2)c1]

GP1 was followed (1.0 mmol scale) to yield compound (156 mg, 0.68 mmol, 68%) as a colourless oil. ¹H NMR (400 MHz, CDCl₃) δ 7.22 (t, J=8.17 Hz, 1H), 6.78-6.27 (m, 4H), 3.88-3.79 (m, 4H), 3.17-3.10 (m, 4H); ¹³C NMR (101 MHz, CDCl₃) δ 152.9, 152.6 (t, J=2.9 Hz), 130.3, 116.3 (t, J=258.5 Hz), 112.3, 109.9, 106.7, 66.8, 48.9; ¹⁹F NMR (377 MHz, CDCl₃) δ −80.2 (d, J=74.3 Hz); HRMS (ESI-TOF): calculated for C₁₁H₁₃F₂NO₂ ([M+H]⁺): 230.0987; found: 230.0988; IR (neat): 2858, 1605, 1582, 1494, 1380, 1352, 1331, 1302, 1265, 1249, 1188, 1114, 1040, 996.

### 2-(4-(difluoromethoxy)phenyl)oxazole

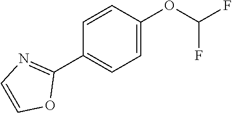 [SMILES: FC(F)Oc1ccc(-c2ncco2)cc1]

GP1 was followed (1.0 mmol scale) to yield compound (166 mg, 0.79 mmol, 79%) as a yellow oil. ¹H NMR (400 MHz, CDCl₃) δ 7.86 (d, J=0.96 Hz, 1H), 7.82 (d, J=0.95 Hz, 1H), 7.65-7.59 (m, 2H), 7.09-7.00 (m, 2H), 6.44 (t, J=73.80 Hz, 1H); ¹³C NMR (101 MHz, CDCl₃) δ 151.4, 151.0 (t, J=2.8 Hz), 139.5, 133.7, 128.1, 127.1, 119.9, 115.8 (t, J=260.1 Hz); ¹⁹F NMR (377 MHz, CDCl₃) δ −80.9 (d, J=73.9 Hz); HRMS (ESI-TOF): calculated for C₁₀H₈F₂NO₂ ([M+H]⁺): 212.0518; found: 212.0518; IR (neat): 1575, 1514, 1502, 1381, 1217, 1177, 1113, 1038, 936, 912, 860.

### 5-(4-(difluoromethoxy)phenyl)-4,6-dimethylpyrimidine

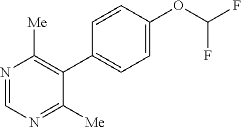 [SMILES: Cc1ncnc(C)c1-c1ccc(OC(F)F)cc1]

GP1 was followed (1.0 mmol scale) to yield compound (59 mg, 0.24 mmol, 24%) as an orange oil. ¹H NMR (400 MHz, CDCl₃) δ 8.92 (s, 1H), 7.34-7.07 (m, 4H), 6.58 (t, J=73.52 Hz, 1H), 2.25 (s, 6H); ¹³C NMR (101 MHz, CDCl₃) δ 164.7, 156.8, 151.0 (t, J=2.8 Hz), 133.6, 133.1, 130.4, 120.2, 115.8 (t, J=260.7 Hz), 23.1; ¹⁹F NMR (377 MHz, CDCl₃) δ −81.1 (d, J=73.7 Hz); HRMS (ESI-TOF): calculated for C₁₃H₁₃ON₂F₂ ([M+H]⁺): 251.0990; found: 251.0990; IR: 1510, 1445, 1393, 1265, 1224, 1265, 1445, 840, 730, 704.

### 6-(difluoromethoxy)quinoline

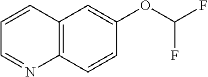 [SMILES: FC(F)Oc1ccc2ncccc2c1]

GP1 was followed (1.0 mmol scale) to yield compound (55 mg, 0.28 mmol, 28%) as a yellow-brown oil. ¹H NMR (400 MHz, CDCl₃) δ 8.89 (dd, J=4.43, 1.68 Hz, 1H), 8.20-8.12 (m, 2H), 7.56-7.49 (m, 2H), 7.45 (dd, J=8.36, 4.32 Hz, 1H), 6.65 (t, J=73.30 Hz, 1H), ¹³C NMR (101 MHz, CDCl₃) δ 149.5, 149.1 (t, J=2.9 Hz), 145.1, 136.4, 131.1, 128.7, 123.7, 121.9, 115.7 (t, J=262.6 Hz) 115.0; ¹⁹F NMR (377 MHz, CDCl₃) δ −81.4 (d, J=73.4 Hz); HRMS (ESI-TOF): calculated for C₁₀H₇F₂NO ([M+H]⁺): 196.0568; found: 196.0569; IR (neat): 1504, 1218, 1171, 1126, 1056, 904, 724.

### 2-(difluoromethoxy)pyridine

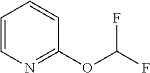

GP1 was followed (1.0 mmol scale) to yield compound (65 mg, 0.45 mmol, 45%) as a pale-yellow oil. ¹H NMR (400 MHz, CDCl₃) δ 8.21 (dd, J=5.0, 2.1 Hz, 1H), 7.77-7.71 (m, 1H), 7.49 (t, J=73.5 Hz, 1H), 7.11 (ddd, J=7.3, 5.0, 0.9 Hz, 1H), 6.91 (dt, J=8.2, 0.9 Hz, 1H); ¹⁹F NMR (377 MHz, CDCl₃) δ −88.86 (d, J=73.2 Hz); MS (ESI-TOF): 146.0 ([M+H]⁺). Spectroscopic data is in accordance with literature.^{[12]}

### 4-(difluoromethoxy)-1-(4-methoxybenzyl)-1H-pyrazolo[3,4-b]pyridine (X.X)

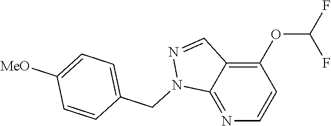 [SMILES: COc1ccc(Cn2ncc3c(OC(F)F)ccnc32)cc1]

GP1 was followed (0.5 mmol scale) to yield compound (105 mg, 0.34 mmol, 68%) as a colourless oil. ¹H NMR (400 MHz, CDCl₃) δ 8.48 (d, J=5.30 Hz, 1H), 8.08 (s, 1H), 7.34-7.30 (m, 2H), 7.01-6.62 (m, 4H), 5.64 (s, 2H), 3.74 (s, 3H); ¹³C NMR (101 MHz, CDCl₃) (159.4, 152.6, 152.3 (t, J=3.3 Hz), 150.4, 129.9, 129.6, 128.9, 115.1 (t, J=263.4 Hz), 114.1, 107.8, 103.3, 55.3, 50.7; ¹⁹F NMR (377 MHz, CDCl₃) δ −83.1 (d, J=72.5 Hz); HRMS (ESI-TOF): calculated for C₁₅H₁₄O₂N₃F₂ ([M+H]⁺): 306.1049; found: 306.1048; IR (neat): 1573, 1514, 1422, 1333, 1265, 1145, 1067, 908, 730, 704, 650.

### 5,7-dichloro-8-(difluoromethoxy)quinoline

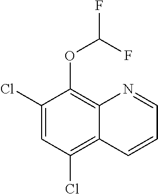 [SMILES: FC(F)Oc1c(Cl)cc(Cl)c2cccnc12]

GP1 was followed (2.0 mmol scale) to yield compound (262 mg, 1.0 mmol, 50%) as an off-white solid. ¹H NMR (500 MHz, CDCl₃) δ 9.01 (dd, J=4.23, 1.62 Hz, 1H), 8.56 (dd, J=8.63, 1.61 Hz, 1H), 7.71 (s, 1H), 7.59 (dd, J=8.57, 4.20 Hz, 1H), 7.34 (t, J=76.28 Hz, 1H); ¹³C NMR (126 MHz, CDCl₃) δ 151.8, 142.9 (t, J=3.8 Hz), 142.4, 133.7, 129.0, 128.2, 128.1, 126.3, 122.7, 117.0 (t, J=263.2 Hz); ¹⁹F NMR (377 MHz, CDCl₃) δ −82.4 (d, J=76.3 Hz); HRMS (ESI-TOF): calculated for C₉H₁₁F₂NO ([M+H]⁺): 263.9789; found: 263.9790; IR (neat): 1603, 1584, 1488, 1458, 1375, 1349, 1081, 1056, 950; MP: 123-124° C.

### 6-chloro-5-(2,3-dichlorophenoxy-1-difluoromethyl)-2-methylthio)-1H-benzo[d]imidazole

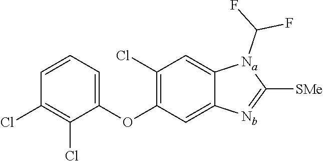

GP2 was followed (1.0 mmol scale) to yield compound (253 mg, 0.62 mmol, 62%) as a mixture of regioisomers (Nₐ/N_{b}: 1:1) as a white solid. ¹H NMR (400 MHz, CDCl₃) δ 7.70-7.58 (m, 1H), 7.31-6.95 (m, 4H), 6.67-6.45 (m, 1H), 2.74 (s, 1.5H), 2.71 (s, 1.5H); ¹³C NMR (101 MHz, CDCl₃) δ 154.7, 154.2, 153.1, 152.9, 148.7 (d, J=3.1 Hz), 147.3, 143.1, 141.3, 134.5 (d, J=4.1 Hz), 131.8, 129.7, 127.6, 127.5, 125.4, 124.8, 124.1, 123.1, 122.9, 121.6, 120.4, 116.9, 115.2, 113.2, 109.9, 108.8 (t, J=250.7 Hz), 104.8 (t, J=2.9 Hz), 15.2, 15.1; ¹⁹F NMR (377 MHz, CDCl₃) δ −95.7 (d, J=58.6 Hz), −95.9 (d, J=58.5 Hz); HRMS (ESI-TOF): calculated for C₁₅H₉C₁₃F₂N₂OS ([M+H]⁺): 408.9547; found: 408.9542; IR (neat): 2921, 1575, 1473, 1445, 1401, 1349, 1316, 1290, 1255, 1192, 1169, 1126, 1087, 1031, 1017, 983, 942, 921, 908, 866, 810; MP: 155-157° C.

### 4-(1-(difluoromethyl)-1H-benzo[d]imidazol-2-yl)thiazole

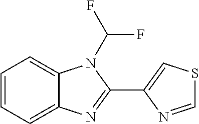 [SMILES: FC(F)n1c(-c2cscn2)nc2ccccc21]

GP2 was followed (1.0 mmol scale) to yield compound (246 mg, 0.98 mmol, 98%) as a light-yellow oil. ¹H NMR (400 MHz, CDCl₃) δ 9.15-8.77 (m, 2H), 8.43 (d, J=2.17 Hz, 1H), 7.85-7.76 (m, 2H), 7.44-7.35 (m, 2H); ¹³C NMR (101 MHz, CDCl₃) δ 153.8, 146.1, 144.6, 142.5, 131.8, 125.1, 124.6, 123.1, 120.0, 113.4 (t, J=3.2 Hz), 110.5 (t, J=249.5 Hz); ¹⁹F NMR (377 MHz, CDCl₃) δ −96.3 (d, J=59.1 Hz); HRMS (ESI-TOF): calculated for C₁₁H₈N₃F₂S ([M+H]⁺): 252.0402; found: 252.0401; IR (neat): 2922, 1636, 1456, 1039, 906, 728, 2922, 1039, 906, 728; MP: 166-167° C.

### N-(5-(difluoromethoxy)-2-phenoxyphenyl)-N-(2,5-dimethoxybenzyl)acetamide

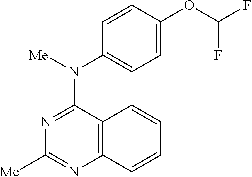 [SMILES: Cc1nc(N(C)c2ccc(OC(F)F)cc2)c2ccccc2n1]

GP1 was followed (0.5 mmol scale) to yield (92 mg, 0.59 mmol, 59%) as a white solid. ¹H NMR (400 MHz, CDCl₃) δ 7.80 (dt, J=8.3, 1.0 Hz, 1H), 7.57 (ddd, J=8.4, 5.1, 3.1 Hz, 1H), 7.20-7.09 (m, 4H), 7.06-6.99 (m, 2H), 6.54 (t, J=73.5 Hz, 1H), 3.62 (s, 3H), 2.75 (s, 3H). ¹³C NMR (101 MHz, CDCl₃) δ 163.37, 161.85, 151.87, 148.73, 145.98, 132.08, 127.68, 126.96, 125.94, 124.39, 121.12, 115.70 (t, J=261.50 Hz), 114.61, 42.40, 26.35. ¹⁹F NMR (377 MHz, CDCl₃) δ −81.11 (d, J=73.7 Hz); HRMS (ESI-TOF): calculated for C₁₇H₁₅F₂N₃O ([M+H]⁺): 316.1256; found: 316.1256. Spectroscopic data is in accordance with literature.^{[13]}.

### 2-(2-(4-(difluoromethoxy)phenyl)-5,7-dimethylpyrazolo[1,5-a]pyrimidin-3-yl)-N,N-diethylacetamide

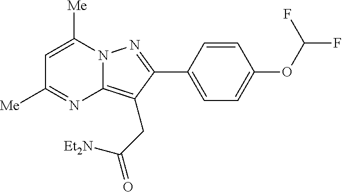

GP1 was followed (0.2 mmol scale) to yield compound (55 mg, 0.14 mmol, 68%) as an off-white solid. ¹H NMR (400 MHz, CDCl₃) δ 7.92-7.83 (m, 2H), 7.23-7.16 (m, 2H), 6.74-6.34 (m, 2H), 3.92 (s, 2H), 3.53 (q, J=7.1 Hz, 2H), 3.40 (q, J=7.1 Hz, 2H), 2.74 (d, J=0.9 Hz, 3H), 2.55 (s, 3H), 1.22 (t, J=7.2 Hz, 3H), 1.10 (t, J=7.1 Hz, 3H); ¹³C NMR (101 MHz, CDCl₃) δ 170.04, 157.86, 154.35, 151.52, 145.10, 131.32, 130.42, 119.56, 116.11 (t, J=259.4 Hz), 108.61, 101.36, 42.50, 40.79, 28.08, 24.70, 17.01, 14.50, 13.20; ¹⁹F NMR (377 MHz, CDCl₃) δ −80.66 (d, J=74.3 Hz); HRMS (ESI-TOF): calculated for C₂₁H₂₅F₂N₄O₂ ([M+H]⁺): 403.1940; found: 403.1941; IR (neat): 2924, 2521, 2159, 2030, 1977, 1643, 1624, 1591, 1481, 1442, 1397, 1229, 1109, 1036; MP: 110-112° C.

### Radiochemistry:

### General Information

[¹⁸F]Fluoride was produced by Alliance Medical (UK) via the ¹⁸O(p,n)¹⁸F reaction and delivered as [¹⁸F]fluoride in O-18-enriched-water. Radiosynthesis and azeotropic drying was performed on a NanoTek microfluidic device (Advion).

### Manual Synthesis of [¹⁸F]tBuPhSO₂CF₂H ([¹⁸F]1)

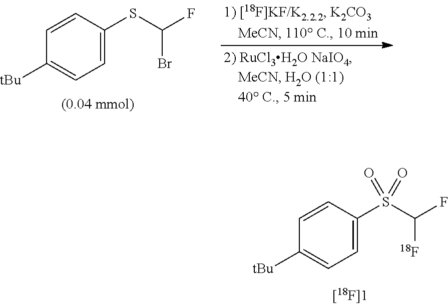 [SMILES: CC(C)(C)c1ccc(SC(F)Br)cc1 ; CC(C)(C)c1ccc(S(=O)(=O)C(F)[18F])cc1]

[¹⁸F]KF/K₂₂₂ capture: [¹⁸F]fluoride was separated from ¹⁸O-enriched-water using an anion exchange cartridge (Waters Sep-Pak AccellPlus QMA Carbonate Plus Light Cartridge, activated with H₂O (10.0 mL) prior to use) and released with a solution of Kryptofix222 (7.5 mg) and K₂CO₃ (1.5 mg) in MeCN/H₂O (0.75 mL, 4:1, v/v). The solution was dried by azeotropic drying using dry MeCN (200 μL) under a flow of N2 at 105° C. (CE1-Temp: 105° C.).

Synthesis of [¹⁸F]tBuPhSO₂CF₂H ([¹⁸F]1): Upon cooling of the v-vial, a solution of (4-(tert-butyl)phenyl)(bromofluoromethyl)sulfane (11.2 mg, 0.04 mmol) in MeCN (dry, 300 μL) was added. The resulting solution was stirred at 110° C. (CE1-Temp: 110° C.) for 10 minutes, after which time it was allowed to cool to 40° C. To the mixture was added RuCl₃×H₂O (2 mg, 20 mol %) and NaIO₄ (52 mg, 0.16 mmol) in MeCN/H₂O (1:1, v/v, 0.5 mL). This mixture was stirred at 35° C. (CE2-Temp: 36° C.) for 10 minutes. The mixture was diluted with a mixture of water (3.2 mL) and EtOH (0.8 mL) and trapped on a C₁₈ plus cartridge (conditioned 10 mL MeOH, then 10 mL H₂O). The mixture was eluted with MeCN (1.5 mL) and loaded onto the HPLC sample-loop for preparative HPLC purification (using isocratic 65% MeCN in 25 mM ammonium formate buffer, Q=4 mL/min, t_{R}([¹⁸F]1)=ca. 11-14 minutes). [¹⁸F]1 was collected in a vial containing H₂O (20 mL), which was shaken and eluted over a C₁₈ Plus cartridge (pre-conditioned with 10 mL MeOH and 10 mL of H₂O). The ¹⁸F-product was released from the C18 Plus cartridge with MeCN (2.0 mL) into a V-vial.

Optimisation of the automated radiosynthesis of [¹⁸F]1 using a Trasis AllInOne synthesizer. Procedure for the optimisation: The reaction was performed with a Trasis AllInOne reactor. The vial in slot 2 was charged with Kryptofix (7.5 mg), K₂CO₃ (0.15 mL, 10 mg/mL aqueous solution) and MeCN (0.85 mL). The vial in slot 9 was charged with tBuPhSCHFBr (11 mg, 0.04 mmol) and dry MeCN (1.0 mL). The vial in slot 10 was charged with NaIO₄ (52 mg), RuCl₃×H₂O (2 mg), water (0.25 mL) and MeCN (0.25 mL). The solvent reservoirs (slots 8 and 17) were filled with dry MeCN (ca 10 mL). The dilution vial (slot 34) was filled with water (10 mL). The QMA carbonate cartridge (slot 5) was activated with water (10 mL) prior to use. The C18 cartridges (slots 26 and 33) were activated with methanol (10 mL) followed by water (10 mL) prior to use.

After drying of fluoride (directly received from cyclotron), the fluorination reaction was run at 110° C. for 10 min. Thereafter, the oxidation reaction was run in the same vessel at 40° C. for 5 min. The crude mixture was then purified by C18 cartridge followed by reverse-phase HPLC (MeCN/H₂O 55:45). The collected product was diluted with water and reformulated via C18 cartridge. An aliquot of the isolated product was analysed by HPLC to determine molar activity.

### ¹⁸F-Difluorocyclopropanation of 1,1-diphenylethylene

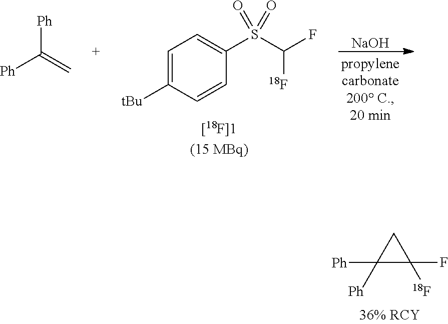

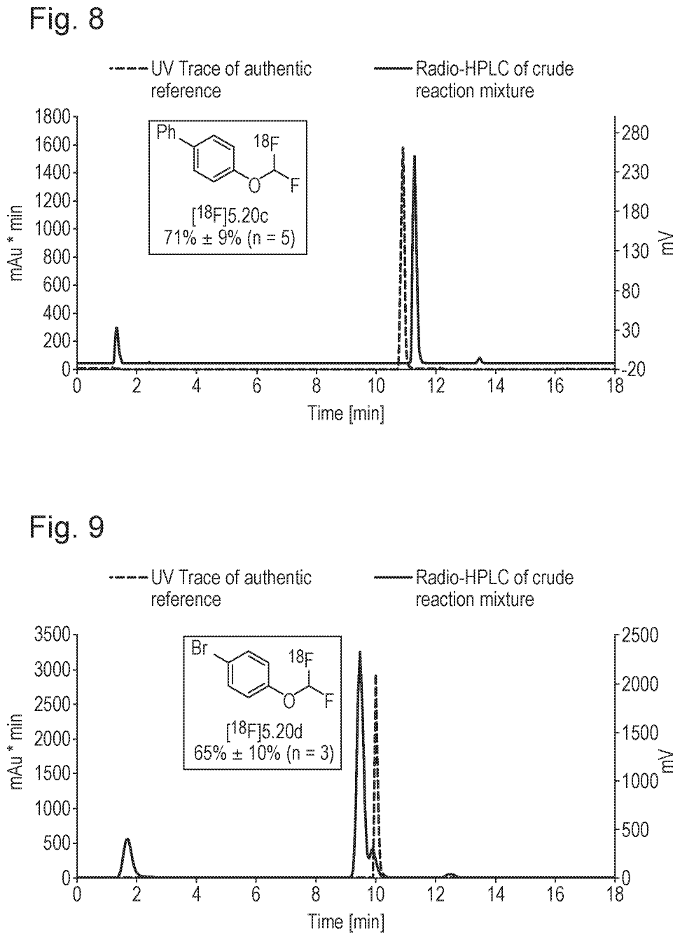

To a mixture of alkene (0.1 mmol) and NaOH (0.05 mmol) in propylene carbonate (300 μL) in a V-vial was added [¹⁸F]1 (15 MBq) in propylene carbonate (20 μL). The mixture was stirred at 200° C. for 20 min. The mixture was cooled to room temperature and quenched by addition of an ethanol solution in water (1:1, 500 μL). An aliquot of this mixture was analysed by radioHPLC (see FIG. 8).

### Optimisation of the Difluoromethylation of Phenols.

Procedure: To a V-vial containing phenol (0.2 mmol) was added [¹⁸F]1 (10-20 MBq) in MeCN (20-50 μL). Then a mixture of MeCN (600 μL) and KOH (25% aq., 100 μL) was added, and the mixture was stirred at 80° C. for 20 min. The mixture was cooled to room temperature and quenched by addition of an ethanol solution in water (1:1, 500 μL). An aliquot of this mixture was further analysed by radioHPLC.

TABLE 1
Optimisation of the reaction conditions for the
¹⁸F-difluoromethylation of phenol

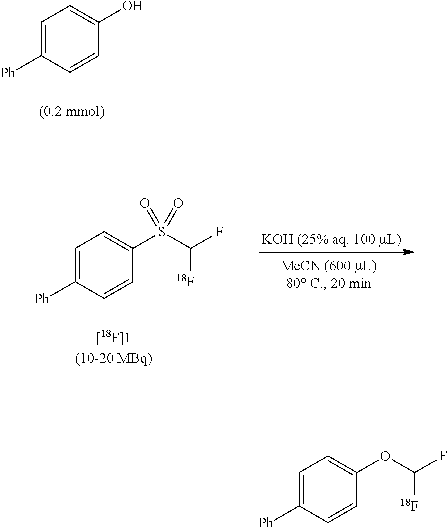 [SMILES: Oc1ccc(-c2ccccc2)cc1 ; O=S(=O)(c1ccc(-c2ccccc2)cc1)C(F)[18F] ; FC([18F])Oc1ccc(-c2ccccc2)cc1]

| Entry | Alterations to conditions | RCY (n = 2) |
|---|---|---|
| 1 | No alteration | 71% ± 9%ª |
| 2 | Phenol (0.1 mmol) | 65% ± 7% |
| 3 | KOH (25% aq, 50 μL) | Trace |
| 4 | KOH (25% aq, 150 μL) | 62% ± 3% |
| 5 | DMSO (600 μL) instead of MeCN | 25% ± 1% |
| 6 | KOH (3 mg) instead of KOH (aq) | 0% |
| 7 | Phenol (0.1 mmol), KOH (25% aq, 50 μL), | 66% ± 14% |
| | MeCN (300 μL) | |

^{a}Reaction performed with n = 5.

### General Procedure 3 (GP3): ¹⁸F-difluoromethylation of (thio)phenols and N-heterocycles

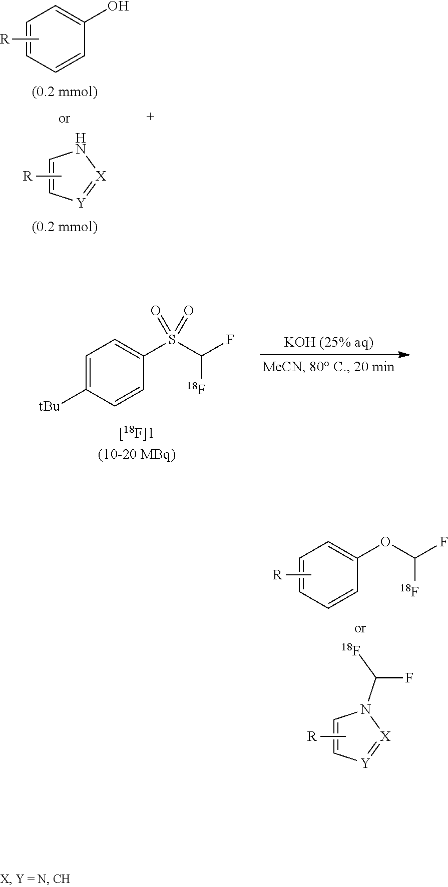

To a V-vial containing (thio)phenol or N-heterocycle (0.2 mmol) was added [¹⁸F]1 in MeCN (20-50 μL). Then a mixture of MeCN (600 μL) and KOH (25% aqueous, 100 μL) was added, and the mixture was stirred at 80 to 100° C. for 20 min. The mixture was cooled to room temperature and quenched by addition of an ethanol solution in water (1:1, 500 μL). An aliquot of this mixture was further analysed by radioHPLC.

### General Procedure 4 (GP4): ¹⁸F-difluoromethylation of (4-methoxyphenyl)methanol

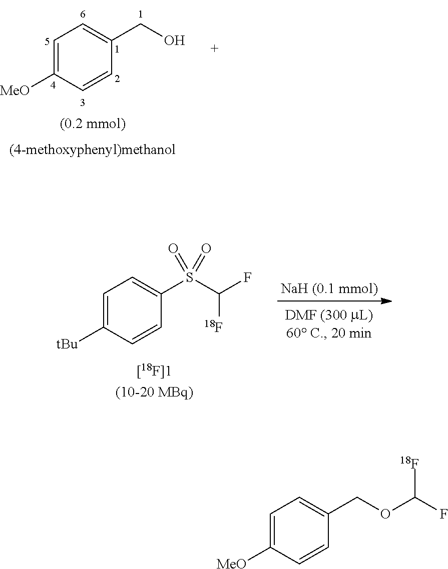 [SMILES: COc1ccc(CO)cc1 ; CC(C)(C)c1ccc(S(=O)(=O)C(F)[18F])cc1 ; COc1ccc(COC(F)[18F])cc1]

To a V-vial containing (4-methoxyphenyl)methanol (0.2 mmol) in DMF (300 μL) was added NaH (0.1 mmol) at 0° C. Then a solution of [¹⁸F]1 in DMF (20-50 μL) was added, and the mixture was stirred at 60° C. for 20 min. The mixture was cooled to room temperature and quenched by addition of an ethanol solution in water (1:1, 500 μL). An aliquot of this mixture was further analysed by radioHPLC.

### General Procedure 5 (GP5): ¹⁸F-difluoromethylation N-heterocycles Under Anhydrous Conditions

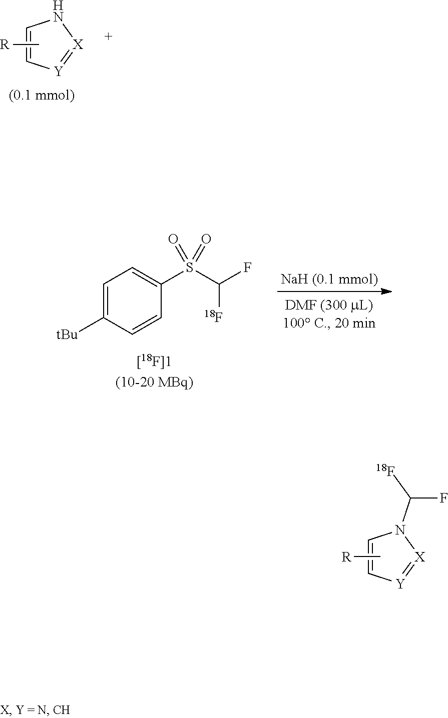

To a V-vial containing N-heterocycle (0.2 mmol) in DMF (300 μL) was added NaH (0.1 mmol) at 0° C. Then a solution of [¹⁸F]1 in DMF (20-50 μL) was added, and the mixture was stirred at 100° C. for 20 min. The mixture was cooled to room temperature and quenched by addition of an ethanol solution in water (1:1, 500 μL). An aliquot of this mixture was further analysed by radioHPLC.

### Competition Experiments

### Benzimidazole vs Phenol

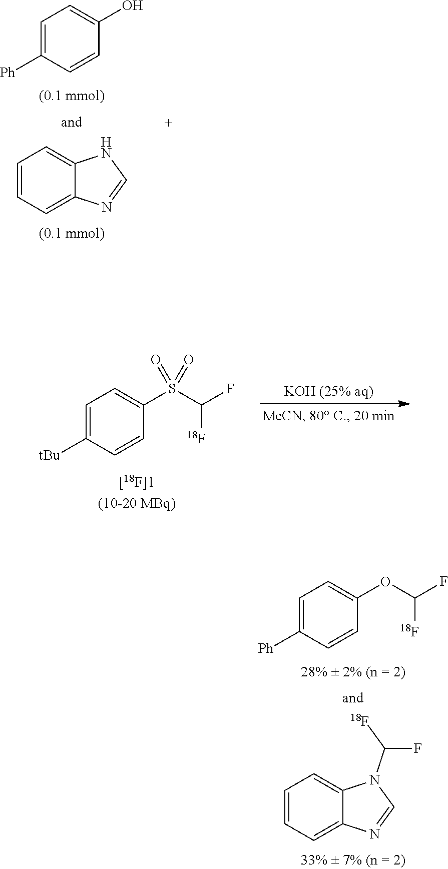 [SMILES: Oc1ccc(-c2ccccc2)cc1 ; c1ccc2[nH]cnc2c1 ; CC(C)(C)c1ccc(S(=O)(=O)C(F)[18F])cc1 ; FC([18F])Oc1ccc(-c2ccccc2)cc1 ; FC([18F])n1cnc2ccccc21]

To a V-vial containing phenol (0.1 mmol) and benzimidazole (0.1 mmol) was added [¹⁸F]1 in MeCN (20-50 μL). Then a mixture of MeCN (600 μL) and KOH (25% aq., 100 μL) was added, and the mixture was stirred at 80° C. for 20 min. The mixture was cooled to room temperature and quenched by addition of an ethanol solution in water (1:1, 500 μL). An aliquot of this mixture was further analysed by radioHPLC.

### Phenol vs Benzylic Alcohol

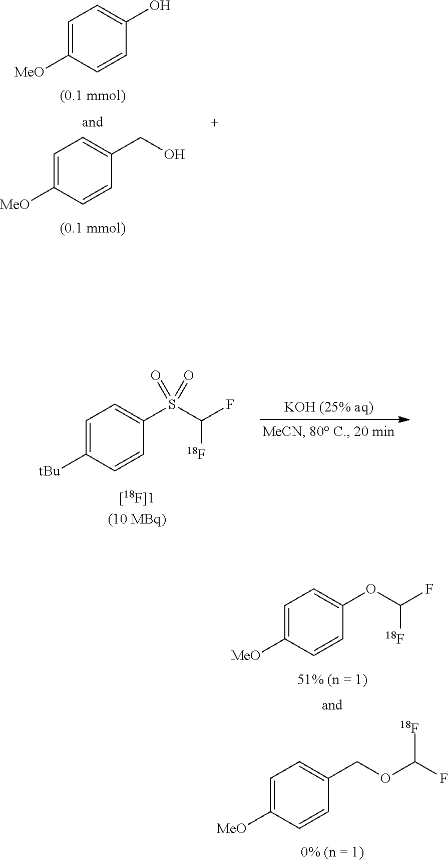 [SMILES: COc1ccc(O)cc1 ; COc1ccc(CO)cc1 ; CC(C)(C)c1ccc(S(=O)(=O)C(F)[18F])cc1 ; COc1ccc(OC(F)[18F])cc1 ; COc1ccc(COC(F)[18F])cc1]

To a V-vial containing phenol (0.1 mmol) and benzylic alcohol (0.1 mmol) was added [¹⁸F]1 in MeCN (20-50 μL). Then a mixture of MeCN (600 μL) and KOH (25% aq., 100 μL) was added, and the mixture was stirred at 80° C. for 20 min. The mixture was cooled to room temperature and quenched by addition of an ethanol solution in water (1:1, 500 μL). An aliquot of this mixture was further analysed by radioHPLC.

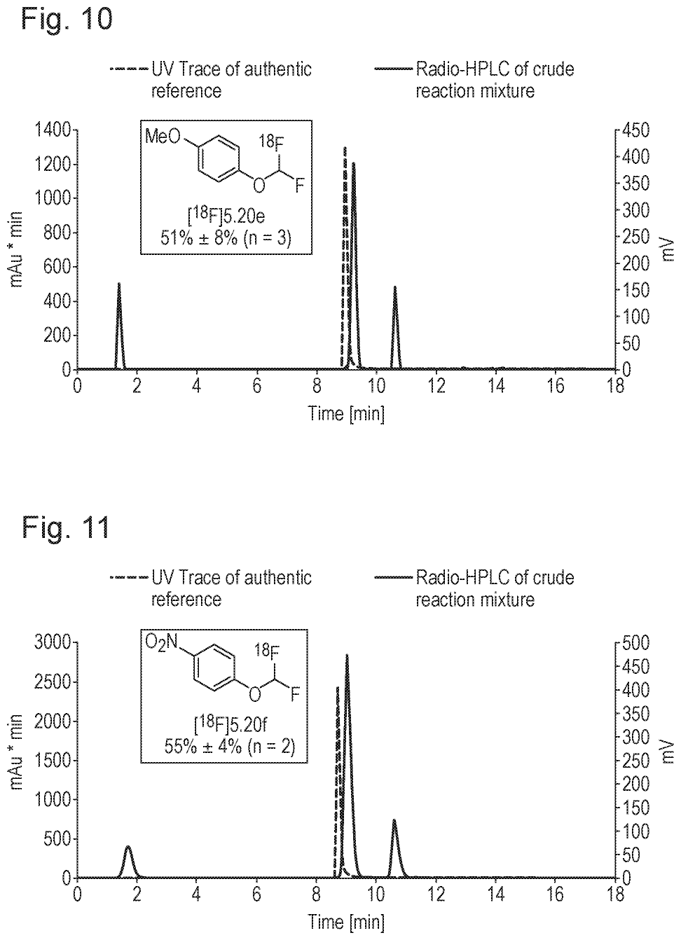

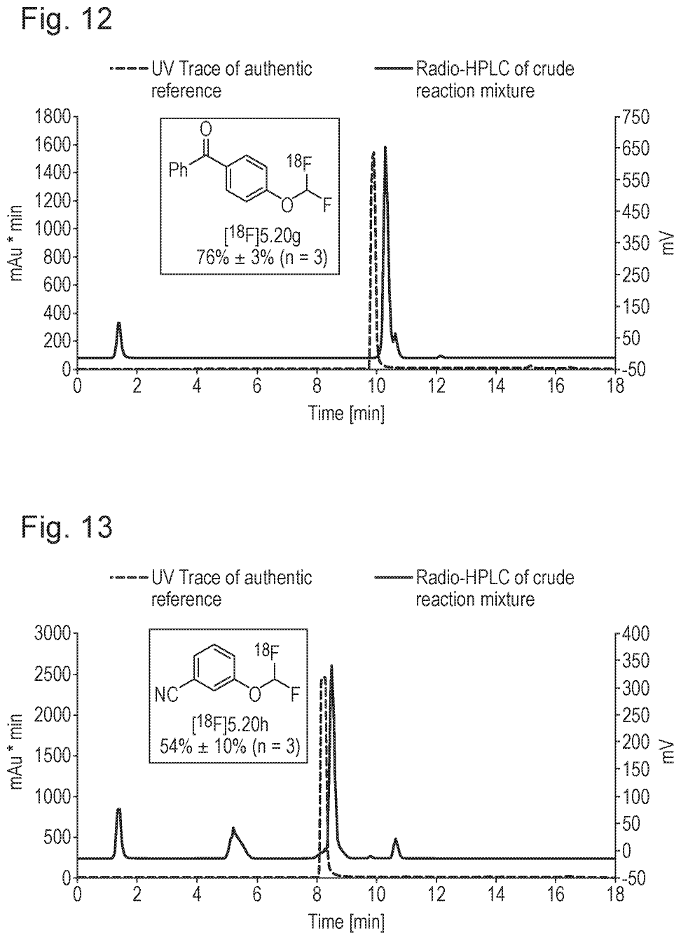

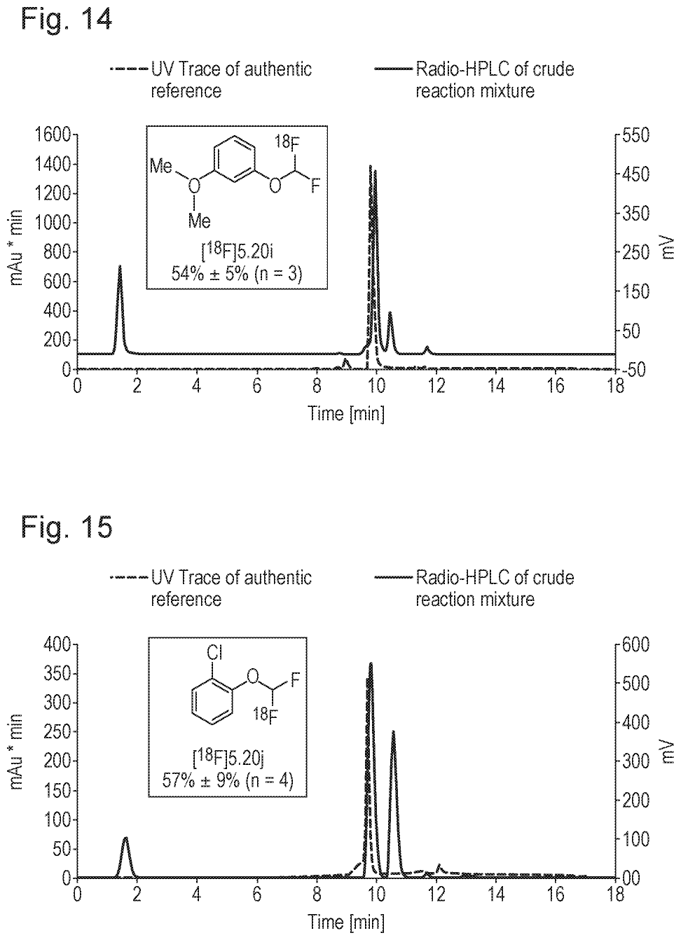

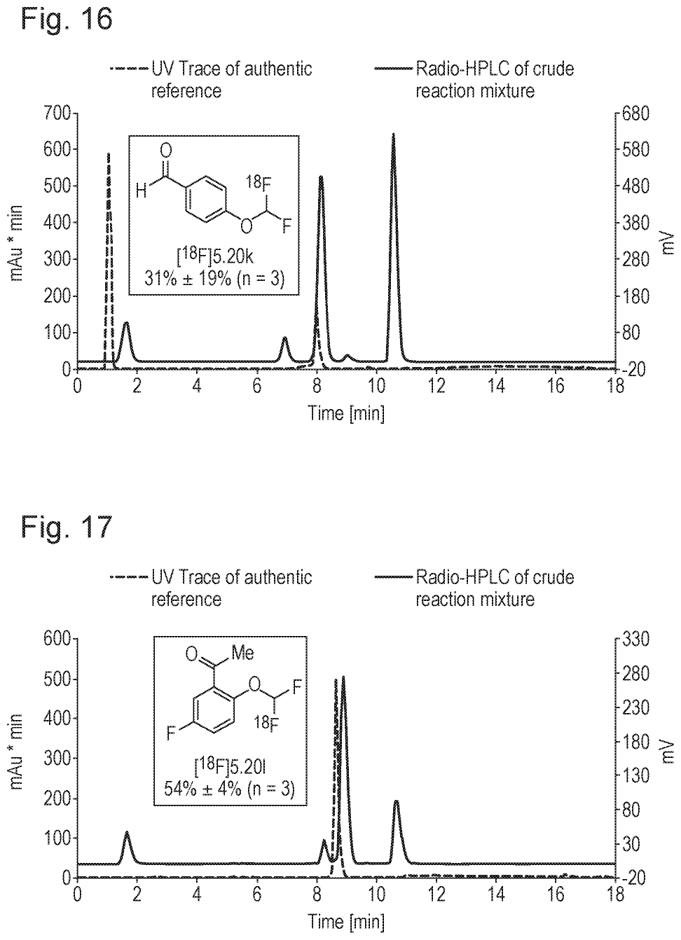

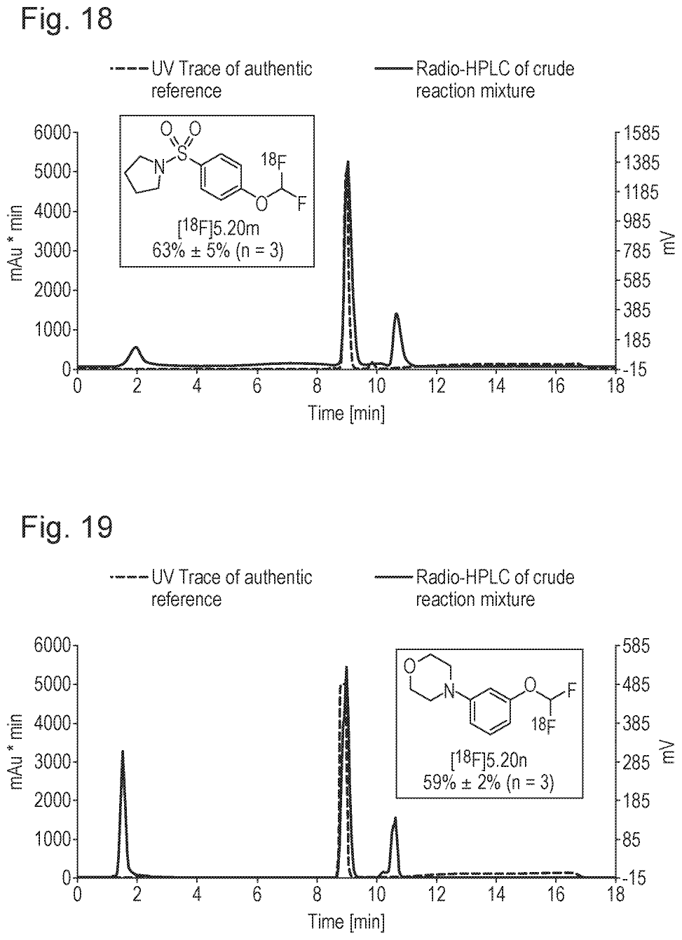

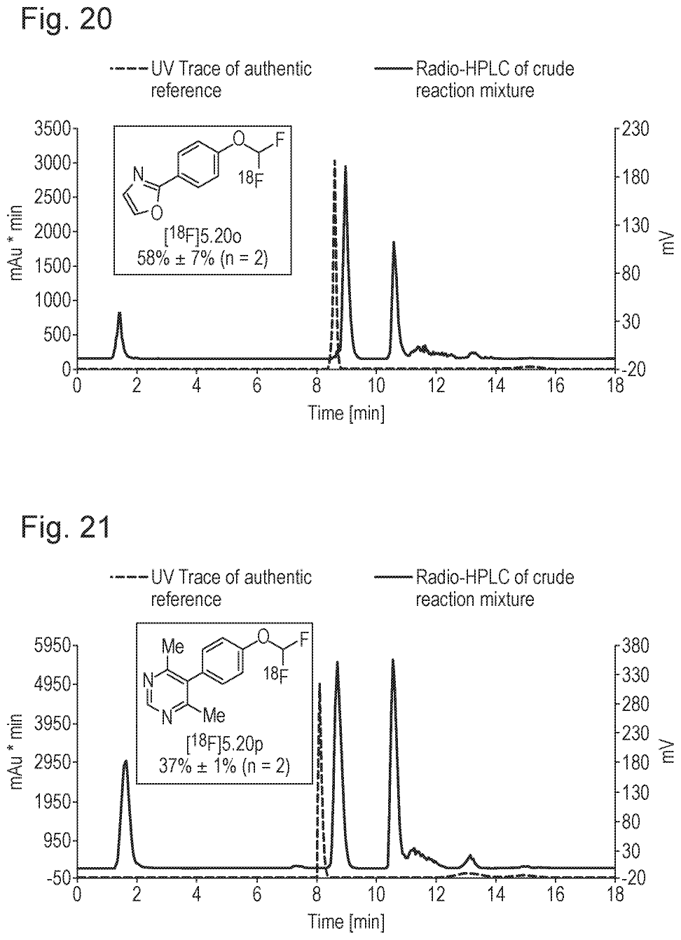

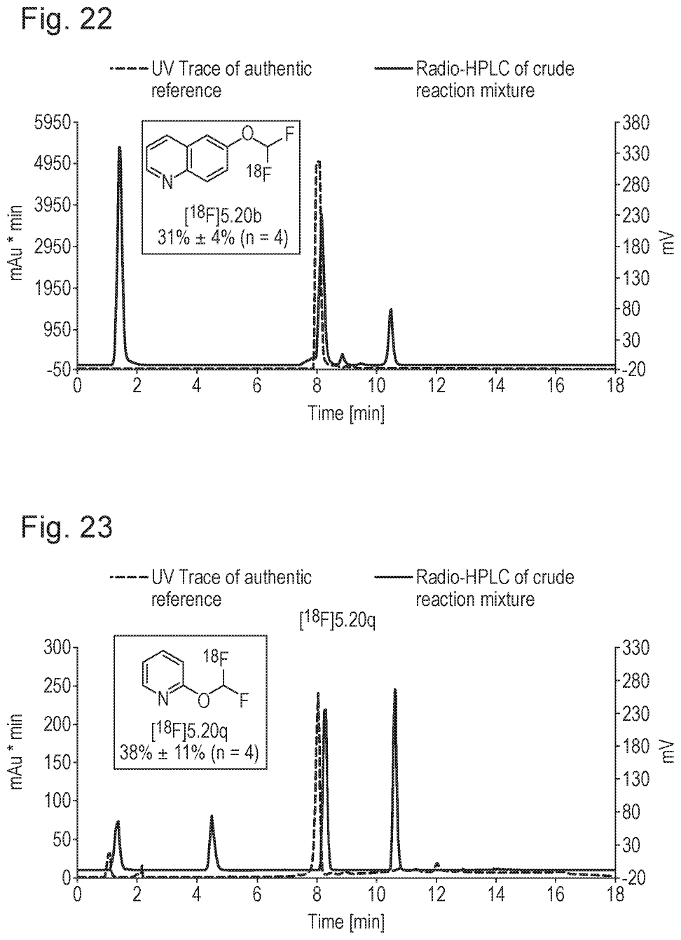

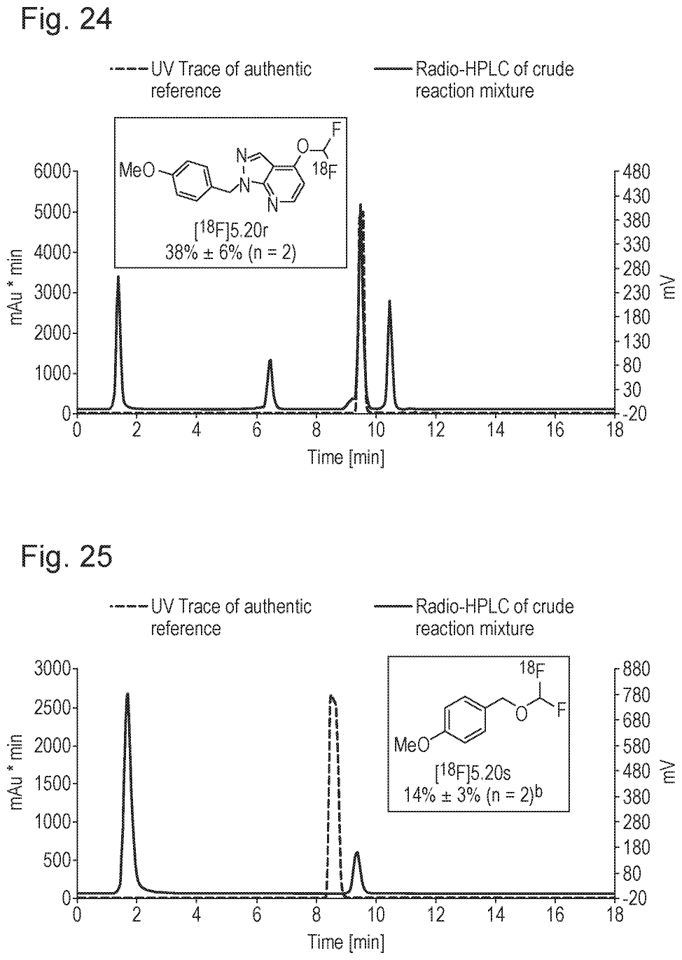

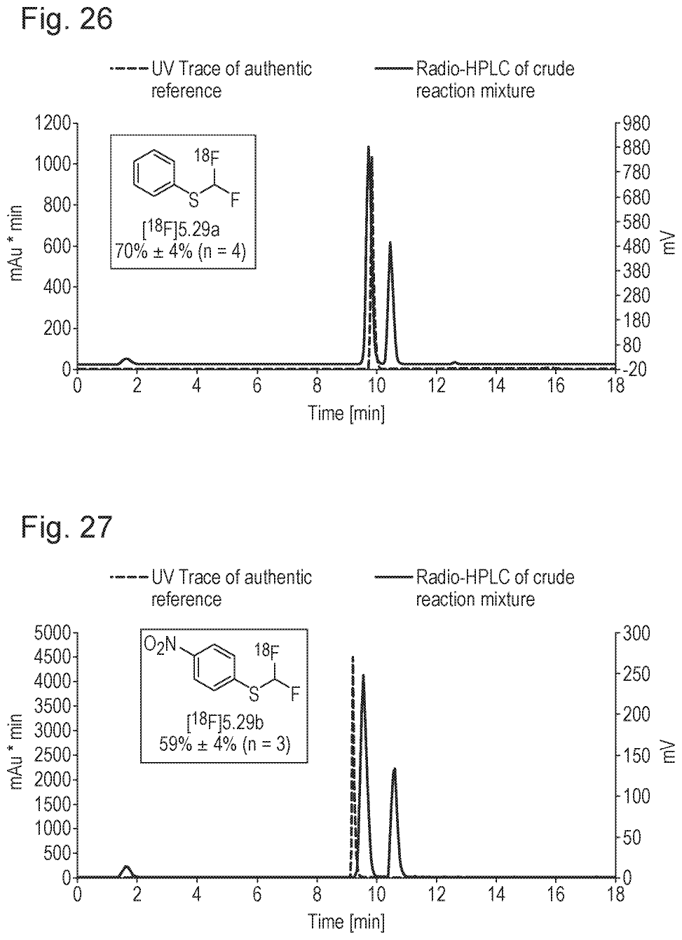

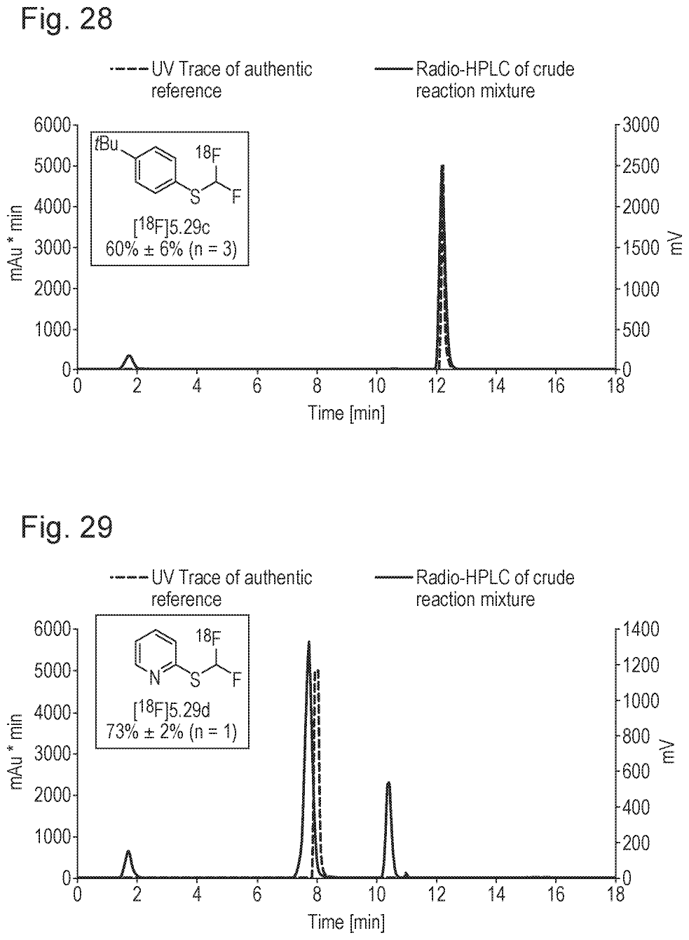

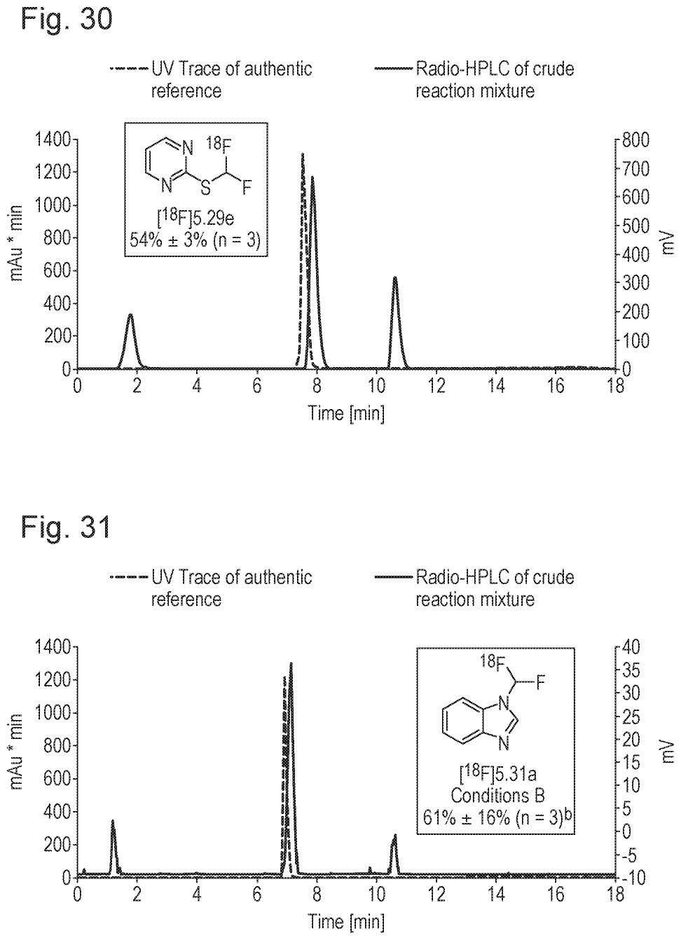

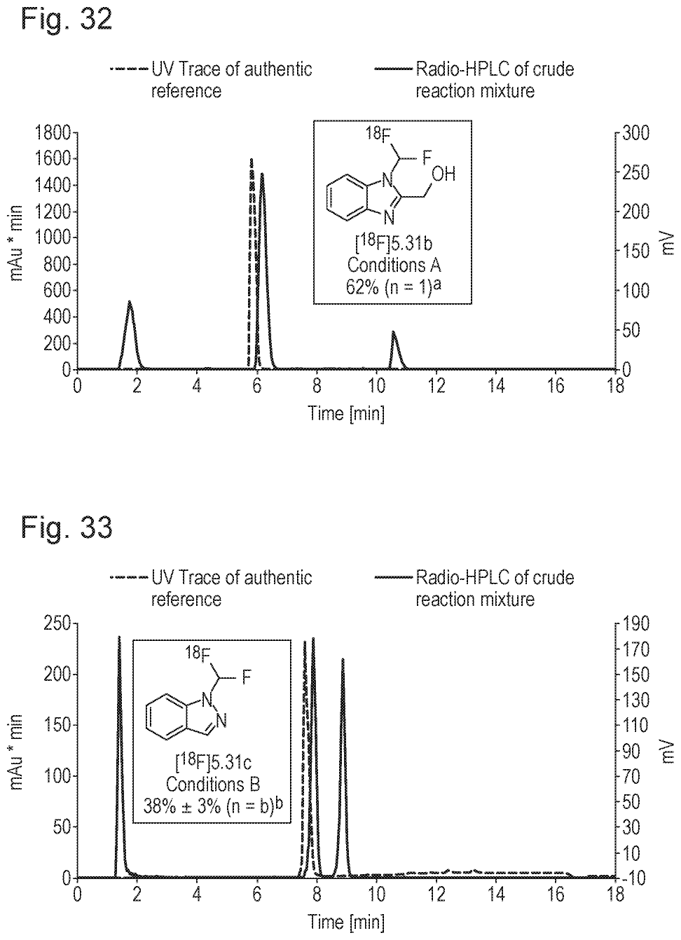

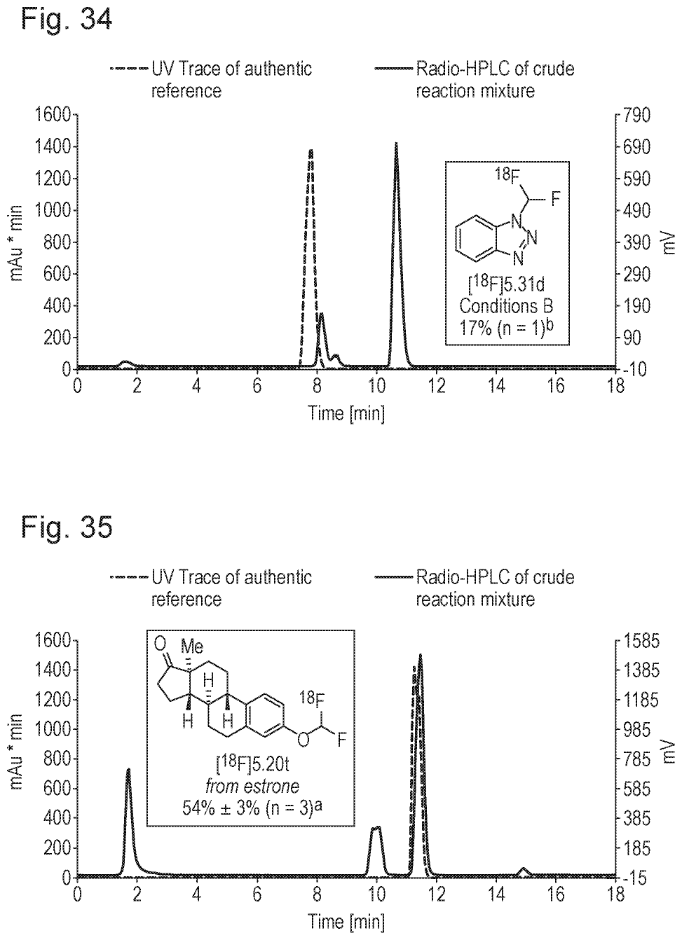

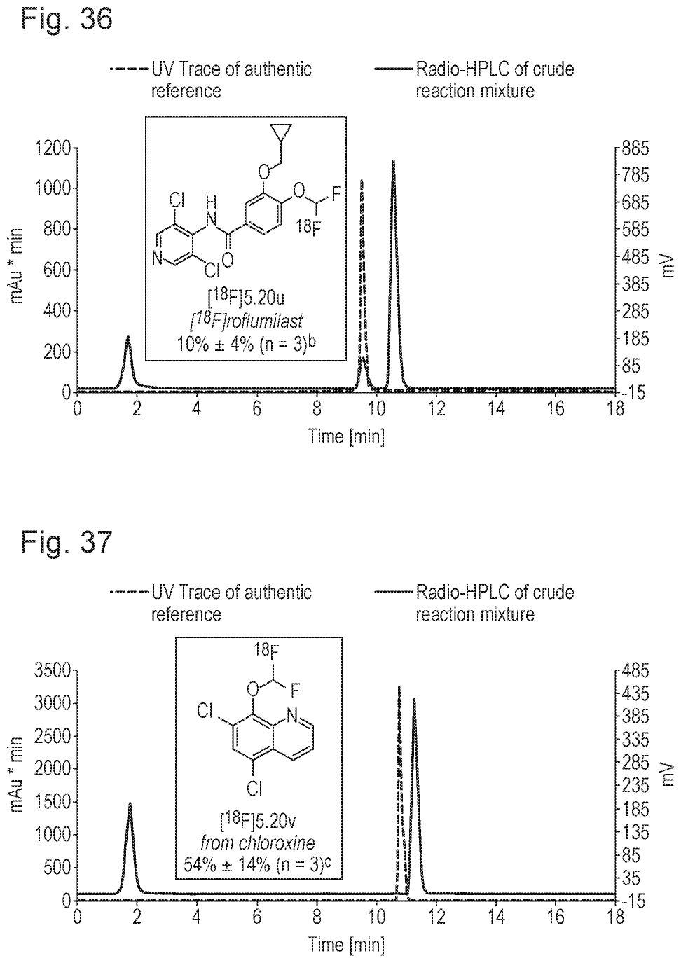

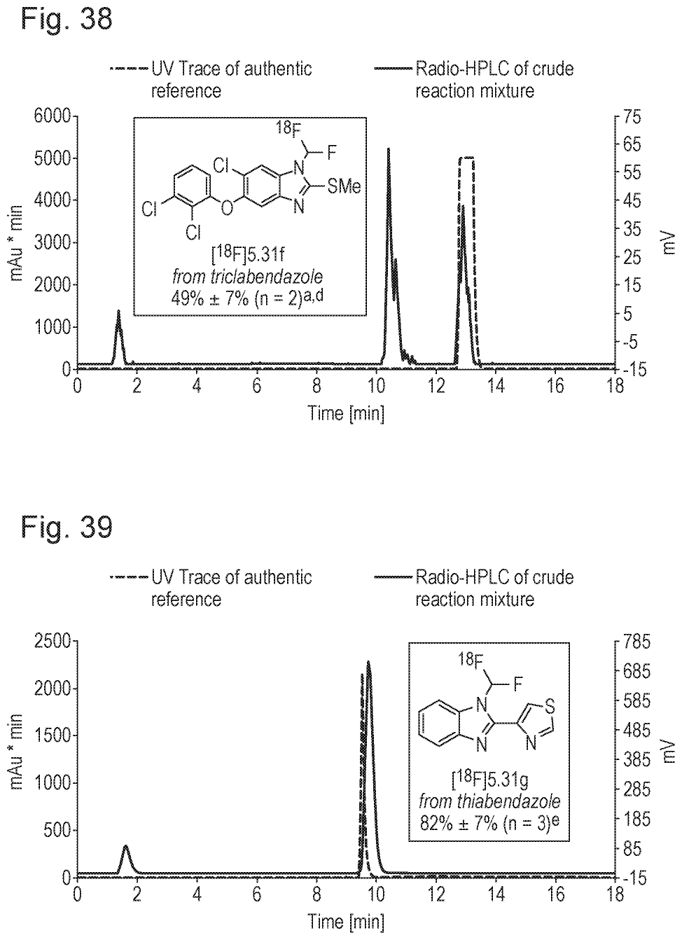

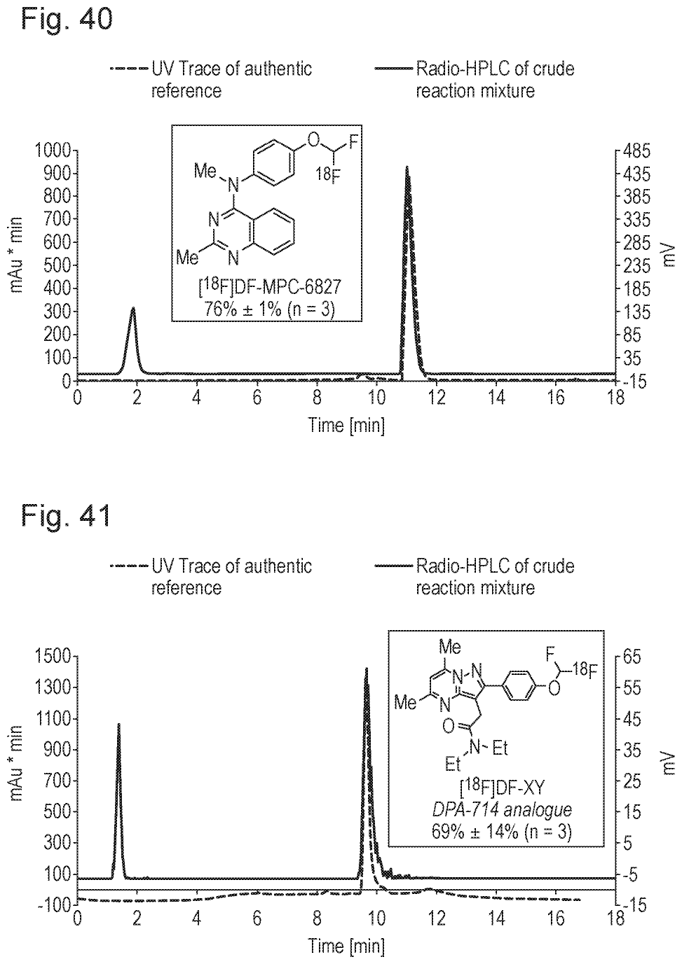

Radio traces for the various compounds synthesized are provided in FIGS. 8 to 41.

### Example 2—Exemplary Reaction Scheme for Reaction of a Compound Comprising a Nucleophilic Group with a Compound of Formula (I)

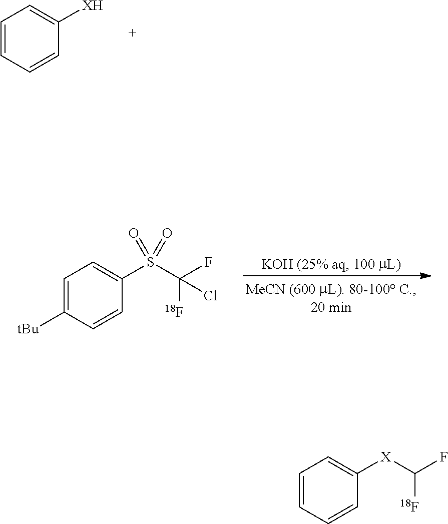

### Example 3—Tandem Procedure from Boronic Acid

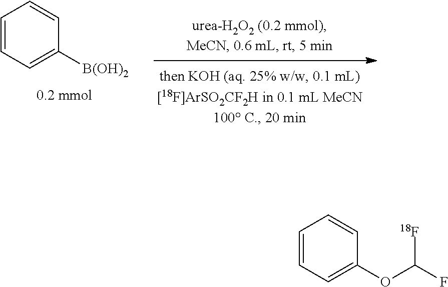 [SMILES: OB(O)c1ccccc1 ; FC([18F])Oc1ccccc1]

In a v-vial, aryl boronic acid (0.2 mmol) was pre-vortexed with Urea*H₂O₂ (0.2 mmol) in 600 μL MeCN for 5 minutes. To the vial was then added KOH_{(aq)} (25%, 0.1 mL) to quench excess peroxide. This crude solution was then added to [¹⁸F]ArSO₂CF₂H in ˜100 μL MeCN. The reaction was then stirred for 20 minutes at 100° C. The reaction mixture was allowed to cool and diluted with 300 μL of a 1:1 (v/v) mixture of EtOH and H₂O. This was filtered into a vial and was subsequently analysed by HPLC. The retention time of the product in a radio-HPLC trace was matched to the UV trace of an authentic reference.

### Example 4—Tandem Procedure from Aryl Chloride

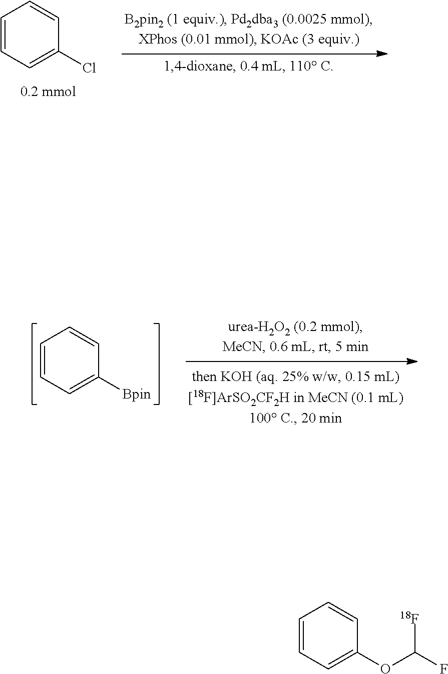

Under air, a vial was charged with fenofibrate (0.2 mmol), B₂pin₂ (0.2-0.6 mmol), KOAc (0.6 mmol), Pd₂dba₃ (0.0025 mmol) and XPhos (0.01 mmol). Degassed 1,4-dioxane (400 μL) was then added and the vial was sealed with a screw cap. This mixture was heated at 110° C. for 20 minutes. Where filtration was employed, the reaction mixture was diluted with EtOAc and passed through a syringe filter. The volatiles were then removed under reduced pressure. The crude mixture was dissolved in 500 μL MeCN and 100 μL DMSO, transferred to a v-vial and then vortexed with Urea*H₂O₂ (0.2 mmol) for 5 minutes to 1 hour. To the vial was then added KOH_{(aq)} (25%, 100-200 μL) to quench excess peroxide. This crude solution was then added to [¹⁸F]ArSO₂CF₂H in ˜100 μL MeCN. The reaction was then stirred for 20 minutes at 100° C. The reaction mixture was allowed to cool and diluted with 300 μL of a 1:1 (v/v) mixture of EtOH and H₂O. This was filtered into a vial and was subsequently analysed by HPLC. The retention time of the product in a radio-HPLC trace was matched to the UV trace of an authentic reference.

### Example 4—Synthesis of ¹⁸F—CF₂H Labelled Etoricoxib from Etoricoxib-B(OH)₂

Reaction conditions: Urea hydrogen peroxide (1.0 equiv), boronic acid in MeCN (0.5 mL), 5 min, rt, then quench KOH_{(aq)} (25% w/w, 0.1 mL), followed by addition of [¹⁸F]ArSO₂CF₂H, 20 min, 100° C.

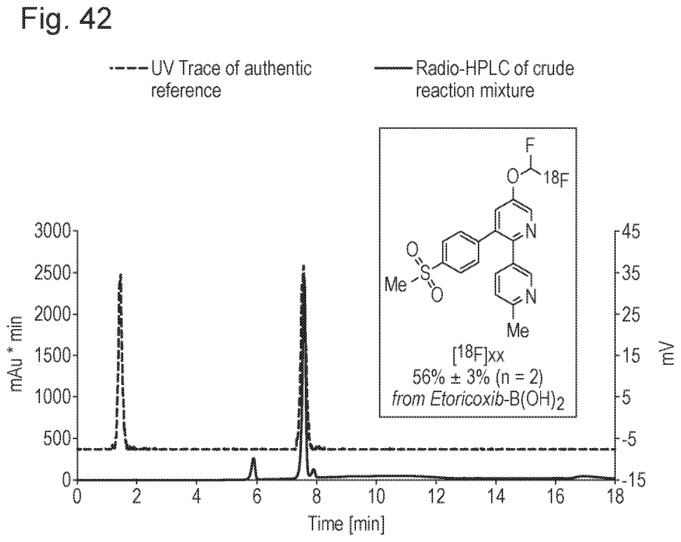

FIG. 42 shows the radio HPLC for the crude reaction mixture for the reaction of Etoricoxib-B(OH)₂.

Further aspects of the invention are set out in the following numbered clauses:
1. A process for producing a compound comprising an ¹⁸F-difluoromethyl group or a compound comprising an ¹⁸F-difluoromethylene group, which process comprises contacting a compound comprising a nucleophilic group with ¹⁸F-difluorocarbene.
2. A process according to clause 1 wherein contacting the compound with ¹⁸F-difluorcarbene comprises treating the compound comprising a nucleophilic group with a source of ¹⁸F-difluorocarbene.
3. A process according to clause 1 or clause 2 wherein the nucleophilic group undergoes a reaction to produce the compound comprising the ¹⁸F-difluoromethyl group or the compound comprising the ¹⁸F-difluoromethylene group.
4. A process for producing a compound comprising an ¹⁸F-difluoromethyl group or a compound comprising an ¹⁸F-difluoromethylene group, which process comprises treating a compound comprising a nucleophilic group with a compound of formula (I) in the presence of a base:

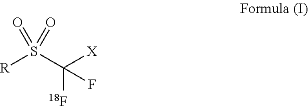

wherein X is H or Cl, preferably wherein X is H, and wherein R is a substituted or unsubstituted aromatic group.
5. A process according to clause 4 wherein the nucleophilic group reacts with the CF ¹⁸F moiety from the compound of formula (I) to produce the compound comprising the ¹⁸F-difluoromethyl group or the compound comprising the ¹⁸F-difluoromethylene group.
6. A process according to any preceding clause wherein the nucleophilic group comprises an atom or ion with a lone pair of electrons or wherein the nucleophilic group comprises a π bond.
7. A process according to any preceding clause wherein the nucleophilic group is XH, wherein X is a heteroatom, or wherein the nucleophilic group is selected from an alkene, an alkyne, a phosphonate, a sulfinate, an aldehyde and a functional group disposed to tautomerism.
8. A process according to any preceding clause wherein the nucleophilic group is selected from the group consisting of OH, SH or NH.
9. A process according to any preceding clause wherein the compound comprising the nucleophilic group comprises an substituted or unsubstituted aromatic group or a substituted or unsubstituted benzylic group, preferably wherein the aromatic group is selected from substituted or unsubstituted aryl and substituted or unsubstituted heteroaryl.
10. A process according to any preceding clause wherein the compound comprising the nucleophilic group comprises a substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl, preferably wherein the compound comprising the nucleophilic group comprises substituted or unsubstituted phenyl.
11. A process according to any one preceding clause wherein the compound comprising the nucleophilic group comprises a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted thiophenyl, substituted or unsubstituted benzofuranyl, substituted or unsubstituted dibenzofuranyl, substituted or unsubstituted benzothiphenyl, substituted or unsubstituted dibenzothiophenyl, substituted or unsubstituted xanthenyl and substituted or unsubstituted carbazolyl; preferably wherein the compound comprising the nucleophilic group comprises substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl and substituted or unsubstituted quinolinyl.
12. A process according to any one of clauses 9 to 11 wherein the nucleophilic group is bonded to the aromatic group.
13. A process according to any preceding clause wherein the compound comprising a nucleophilic group is a compound of formula (II):

Ar-L-Nuc Formula (II)

wherein Ar represents a substituted or unsubstituted aromatic group, wherein the aromatic group is as defined in any one of clauses 9 to 11, L represents a linking group or a bond, and wherein Nuc represents a nucleophilic group as defined in any one of clauses 6 to 8.
14. A process according to clause 13 wherein L is selected from a bond or an unsubstituted C₁a alkylene.
15. A process according to clause 13 or clause 14 wherein Nuc represents an OH or SH group.
16. A process according to clause 13 or clause 14 wherein Nuc is NH and the compound comprising a nucleophilic group comprises an N-heterocyclic ring wherein the nitrogen atom of Nuc is a ring atom in the heterocyclic ring
17. A process according to clause 16 wherein N-heterocyclic ring is in a N-containing heteroaryl, optionally wherein the N-heterocyclic ring is selected from the group consisting of substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl and substituted or unsubstituted quinolinyl.
18. A process according to any one of clauses 4 to 17 wherein the compound of formula (I) provides ¹⁸F-difluorocarbene via an alpha elimination reaction.
19. A process according to any one of clauses 4 to 18 wherein R is selected from substituted or unsubstituted aryl and substituted or unsubstituted heteroaryl.
20. A process according to any one of clauses 4 to 19 wherein R is a substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl, preferably wherein R is substituted or unsubstituted phenyl.
21. A process according to any one of clauses 4 to 19 wherein R is a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted benzothiazolyl and substituted or unsubstituted carbazolyl; preferably wherein R is substituted or unsubstituted pyridyl, substituted or unsubstituted benzothiazolyl or substituted or unsubstituted pyrimidyl.
22. A process according to any one of clauses 4 to 20 wherein the compound of formula (I) is a compound having the following formula:

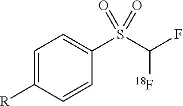

wherein R is selected from the group consisting of ^{t}Bu, nitro and Cl, preferably wherein R is Cl.
23. A process according to any one of clauses 1 to 3 wherein the source of ¹⁸F-difluorocarbene is a compound of formula (I) as defined in any one of clauses 4 and 18 to 23.
24. A process according to clause 23 wherein the ¹⁸F-difluorocarbene is generated by treating the compound of formula (I) with a base.
25. A process according to any one of clauses 4 to 22 and 24 wherein the base is selected from metal hydroxides and metal hydrides, preferably wherein the base is selected from NaOH, KOH and NaH.
26. A process according to any one of clauses 2 to 25 wherein treating the compound comprising a nucleophilic group is performed in the presence of a solvent, preferably wherein the solvent is a polar aprotic solvent.
27. A process according to any preceding clause wherein treating the compound comprising a nucleophilic group is performed at a temperature of at least 40° C., preferably wherein treating the compound comprising a nucleophilic group is performed at a temperature of between 50° C. and 250° C.
28. A process according to any preceding clause wherein the compound comprising a ¹⁸F-difluoromethyl group or the compound comprising an ¹⁸F-difluoromethylene group is a positron emission tomography (PET) ligand.
29. A process according to any preceding clause wherein the compound comprising a ¹⁸F-difluoromethyl group is an ¹⁸F-difluoromethyl functionalised microtubule ligand, an ¹⁸F-difluoromethyl functionalised microtubule ligand PK-M2 ligand, an ¹⁸F-difluoromethyl functionalised microtubule ligand P2X₇R ligand, an ¹⁸F-difluoromethyl functionalised microtubule ligand mGluR2 ligand, an ¹⁸F-difluoromethyl functionalised TSPO ligand or an ¹⁸F-difluoromethyl functionalised GABAA receptor antagonist.
30. A compound of formula (I)

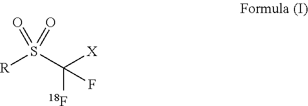

wherein X is H or Cl, preferably wherein X is H, and wherein R is a substituted or unsubstituted aryl group.
31. A compound according to clause 30 wherein R is a substituted or unsubstituted phenyl group.
32. A compound according to clause 30 or 31 wherein the compound of formula (I) is a compound having the following formula:

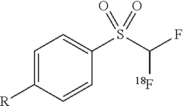

wherein R is selected from the group consisting of tBu, nitro and Cl, preferably wherein R is Cl.
33. A compound comprising an ¹⁸F-difluoromethyl group bonded to nitrogen, optionally wherein the compound is selected from the following compounds:

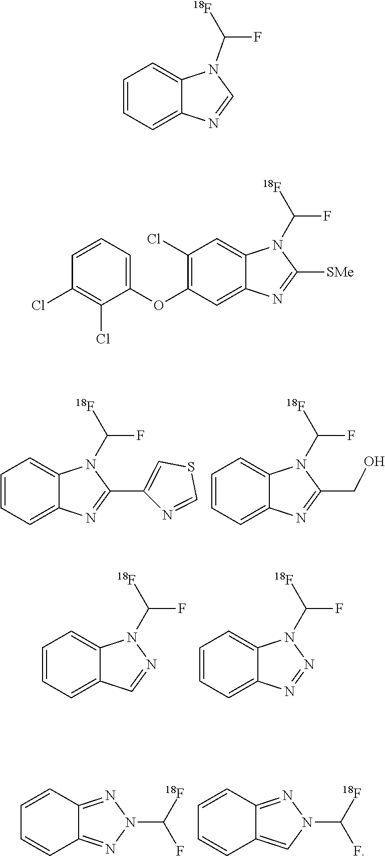

34. A compound comprising a gem-¹⁸F-difluorocyclopropane moiety, optionally wherein the compound is

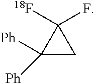 [SMILES: FC1([18F])CC1(c1ccccc1)c1ccccc1]

35. A compound comprising an ¹⁸F-difluoromethyl group bonded to an alkyne.
36. A compound comprising an ¹⁸F-difluoromethyl group bonded to an oxygen atom, optionally wherein the compound is selected from the following compounds:

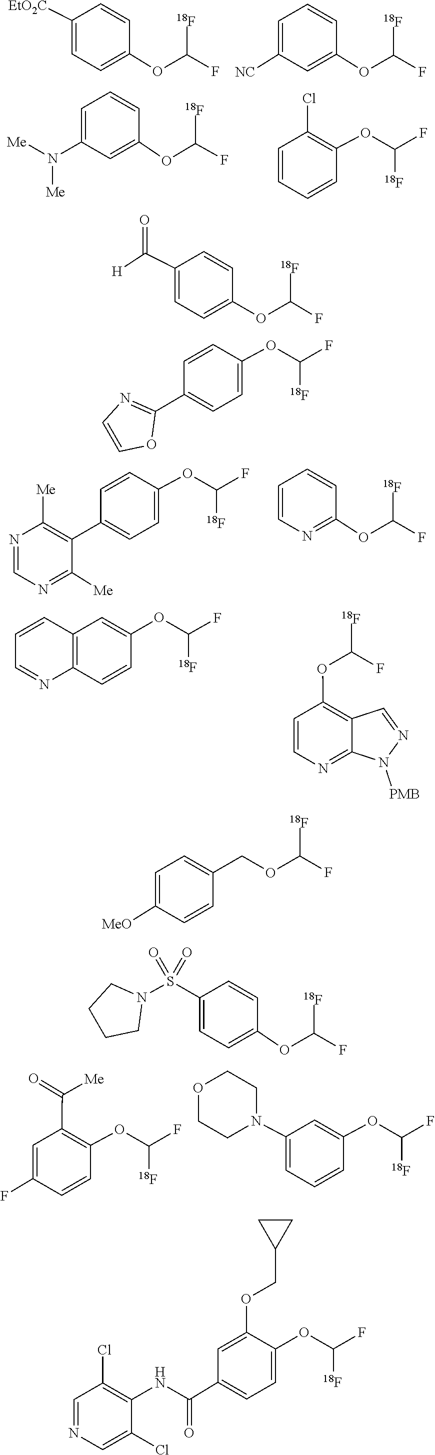

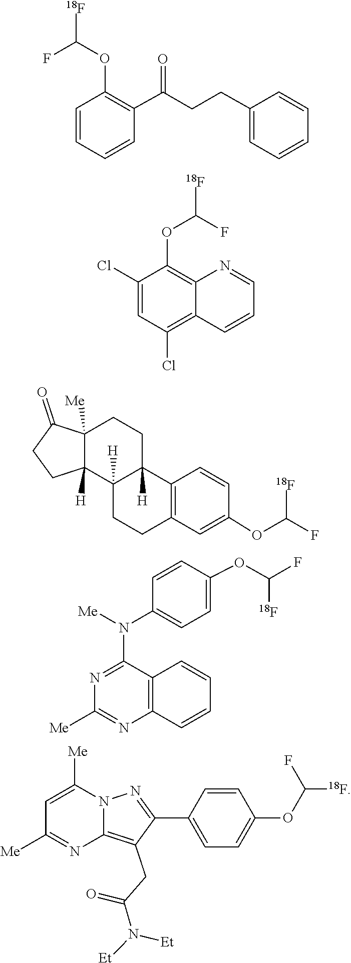

37. A compound comprising an ¹⁸F-difluoromethyl group bonded to a sulfur atom, optionally wherein the compound is selected from the following compounds:

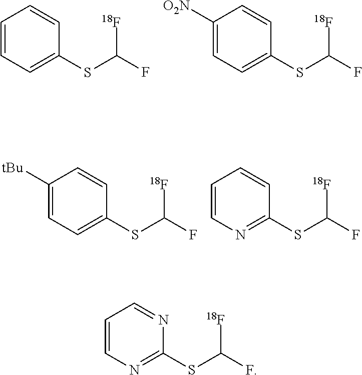

38. A compound according to any one of clauses 33 to 37 wherein the compound is a positron emission tomography (PET) ligand.
39. A compound according to any one of clauses 33 to 37 wherein the compound is obtainable by the process as defined in any one of clauses 1 to 29.
40. A compound as defined in any one of clauses 33 to 39 for use in a method for treatment of the human or animal body by therapy or for use in a diagnostic method practised on the human or animal body.
41. A method of imaging a subject, comprising administering to the subject a compound as defined in any one of clauses 33 to 39 or a pharmaceutically acceptable salt thereof, and imaging the subject by positron emission tomography (PET).

1. A process for producing a compound comprising an ¹⁸F-difluoromethyl group or a compound comprising an ¹⁸F-difluoromethylene group, which process comprises contacting a compound comprising a nucleophilic group with ¹⁸F-difluorocarbene.

2. A process according to claim 1 wherein the nucleophilic group undergoes a reaction to produce the compound comprising the ¹⁸F-difluoromethyl group or the compound comprising the ¹⁸F-difluoromethylene group.

3. A process for producing a compound comprising an ¹⁸F-difluoromethyl group or a compound comprising an ¹⁸F-difluoromethylene group, which process comprises treating a compound comprising a nucleophilic group with a compound of formula (I) in the presence of a base: 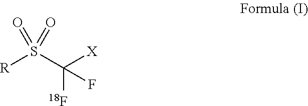
wherein X is H or Cl, preferably wherein X is H, and
wherein R is a substituted or unsubstituted aromatic group,
preferably wherein the nucleophilic group reacts with the CF⁸F moiety from the compound of formula (I) to produce the compound comprising the ¹⁸F-difluoromethyl group or the compound comprising the ¹⁸F-difluoromethylene group.

4. A process according to claim 1 wherein the nucleophilic group comprises an atom or ion with a lone pair of electrons or wherein the nucleophilic group comprises a t bond, preferably wherein the nucleophilic group is XH, wherein X is a heteroatom, or wherein the nucleophilic group is selected from an alkene, an alkyne, a phosphonate, a sulfinate, an aldehyde and a functional group disposed to tautomerism. more preferably wherein the nucleophilic group is selected from the group consisting of OH, SH or NH.

5. A process according to claim 1 wherein the compound comprising the nucleophilic group comprises an substituted or unsubstituted aromatic group or a substituted or unsubstituted benzylic group,
preferably wherein the aromatic group is selected from substituted or unsubstituted aryl and substituted or unsubstituted heteroaryl.

6. A process according to claim 5 wherein the nucleophilic group is bonded to the aromatic group.

7. A process according to claim 4 wherein the compound comprising a nucleophilic group is a compound of formula (II):
Ar-L-Nuc Formula (II)
wherein Ar represents a substituted or unsubstituted aromatic group,
L represents a linking group or a bond, and wherein Nuc represents said nucleophilic group, preferably wherein L is selected from a bond or an unsubstituted C₁₋₄ alkylene.

8. A process according to claim 7 wherein Nuc represents an OH or SH group or wherein Nuc is NH and the compound comprising a nucleophilic group comprises an N-heterocyclic ring wherein the nitrogen atom of Nuc is a ring atom in the heterocyclic ring, preferably wherein the N-heterocyclic ring is in a N-containing heteroaryl,
optionally wherein the N-heterocyclic ring is selected from the group consisting of substituted or unsubstituted imidazolyl, substituted or unsubstituted pyridinyl, substituted or unsubstituted pyrimidinyl, substituted or unsubstituted benzimadazolyl, substituted or unsubstituted indazolyl, substituted or unsubstituted benzotriazolyl and substituted or unsubstituted quinolinyl.

9. A process according to claim 3 wherein the compound of formula (I) provides ¹⁸F-difluorocarbene via an alpha elimination reaction.

10. A process according to claim 3 wherein R is selected from substituted or unsubstituted aryl and substituted or unsubstituted heteroaryl,
preferably wherein
(i) R is a substituted or unsubstituted aryl group selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl,
preferably wherein R is substituted or unsubstituted phenyl; or
(ii) wherein R is a substituted or unsubstituted heteroaryl group selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, substituted or unsubstituted benzothiazolyl and substituted or unsubstituted carbazolyl;
preferably wherein R is substituted or unsubstituted pyridyl, substituted or unsubstituted benzothiazolyl or substituted or unsubstituted pyrimidyl.

11. A process according to claim 3 wherein the compound of formula (I) is a compound having the following formula: 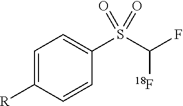
wherein R is selected from the group consisting of ^{t}Bu, nitro and Cl,
preferably wherein R is Cl.

12. A process according to claim 1 wherein the source of ¹⁸F-difluorocarbene is a compound of formula (I): 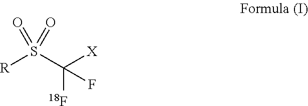
wherein X is H or Cl, and
wherein R is a substituted or unsubstituted aromatic group.

13. A process according to claim 12 wherein the ¹⁸F-difluorocarbene is generated by treating the compound of formula (I) with a base,
preferably wherein the base is selected from metal hydroxides and metal hydrides,
more preferably wherein the base is selected from NaOH, KOH and NaH.

14. A process according to claim 1 wherein the compound comprising a ¹⁸F-difluoromethyl group or the compound comprising an ¹⁸F-difluoromethylene group is a positron emission tomography (PET) ligand, and/or wherein the compound comprising a ¹⁸F-difluoromethyl group is an ¹⁸F-difluoromethyl functionalised microtubule ligand, an ¹⁸F-difluoromethyl functionalised microtubule ligand PK-M2 ligand, an ¹⁸F-difluoromethyl functionalised microtubule ligand P2X₇R ligand, an ¹⁸F-difluoromethyl functionalised microtubule ligand mGluR2 ligand, an ¹⁸F-difluoromethyl functionalised TSPO ligand or an ¹⁸F-difluoromethyl functionalised GABAA receptor antagonist.

15. A compound of formula (I) 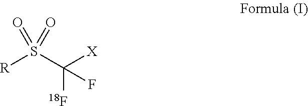
wherein X is H or Cl, preferably wherein X is H, and
wherein R is a substituted or unsubstituted aryl group.

16. A compound according to claim 15 wherein the compound of formula (I) is a compound having the following formula: 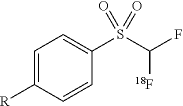
wherein R is selected from the group consisting of ^{t}Bu, nitro and Cl, preferably wherein R is Cl.

17. A compound comprising
(i) an ¹⁸F-difluoromethyl group bonded to nitrogen, or
(ii) a gem-¹⁸F-difluorocyclopropane moiety; or
(iii) an ¹⁸F-difluoromethyl group bonded to an alkyne; or
(iv) an ¹⁸F-difluoromethyl group bonded to an oxygen atom; or
(v) an ¹⁸F-difluoromethyl group bonded to a sulfur atom.

18. (canceled)

19. (canceled)

20. (canceled)

21. (canceled)

22. A compound according to claim 17 wherein the compound is a positron emission tomography (PET) ligand.

23. A compound according to claim 17 wherein the compound is obtainable by a process comprising contacting a compound comprising a nucleophilic group with ¹⁸F-difluorocarbene.

24. A method of treatment comprising administering a therapeutically effective amount of a compound as defined in claim 17 or a pharmaceutically acceptable salt thereof to a subject.

25. A method of imaging a subject, comprising administering to the subject a compound as defined in claim 17 or a pharmaceutically acceptable salt thereof, and imaging the subject by positron emission tomography (PET).

26. A compound according to claim 17, wherein the compound is selected from the following compounds: 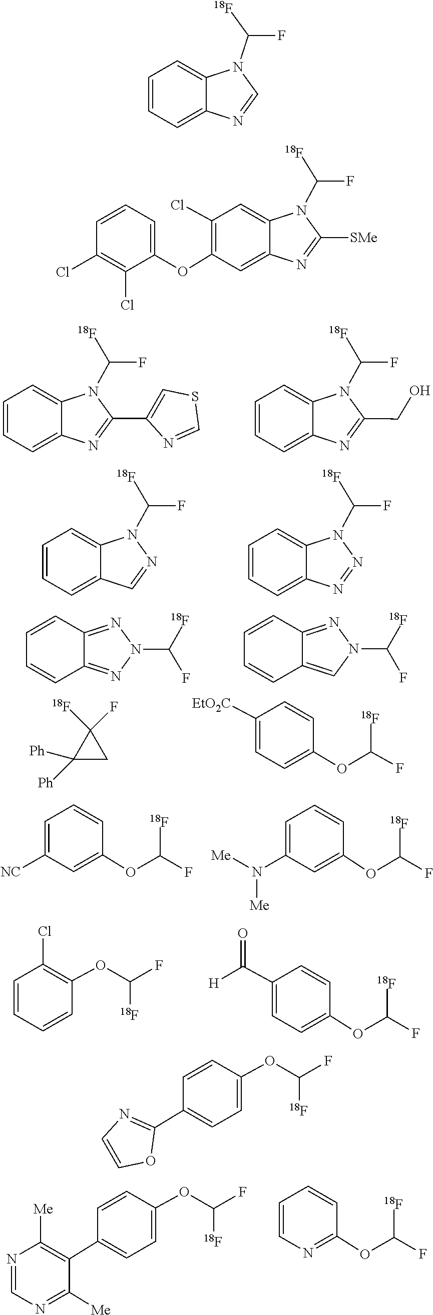 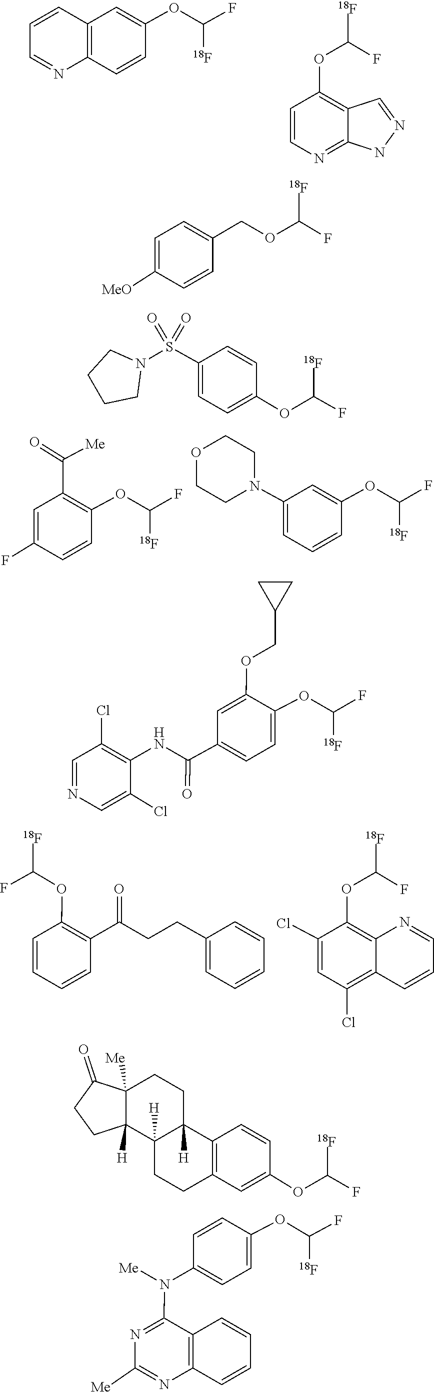 [chemical structure]
